(19) United States
(12) Patent Application Publication
(10) Pub. No.: US 2019/0142800 A1
(43) Pub. Date: May 16, 2019

(54) SYNERGISTIC ANTIFUNGAL COMPOSITIONS AND METHODS THEREOF

(71) Applicant: VYOME THERAPEUTICS LIMITED, DELHI (IN)

(72) Inventors: Shamik GHOSH, Andover, MA (US); Sumana GHOSH, Delhi (IN); Mau SINHA, Uttar Pradesh (IN); Suresh SADHASIVAM, Tamil Nadu (IN); Anubhuti JAIN, Madhya Pradesh (IN); Anamika BHATTACHARYYA, Delhi (IN)

(73) Assignee: VYOME THERAPEUTICS LIMITED, DELHI (IN)

(21) Appl. No.: 16/309,751

(22) PCT Filed: Jun. 13, 2017
(86) PCT No.: PCT/IB2017/053505

§ 371 Date: Dec. 13, 2018

Related U.S. Application Data
(60) Provisional application No. 62/349,438, filed on Jun. 13, 2016.

(30) Foreign Application Priority Data
Aug. 12, 2016 (IN) 201611027707
Feb. 17, 2017 (IN) 201711005689

(51) Int. Cl. A61K 31/4015; A61K 45/06; A61P 31/10

(52) U.S. Cl. CPC A61K 31/4015; A61P 31/10; A61K 45/06

(57) ABSTRACT

The present invention relates to the field of antimicrobials and pharmaceutical sciences. The invention provides anti-fungal compositions for the management of fungal growth and treatment of fungal infections, including treatment of resistant fungal infections. The present compositions comprise at least one antifungal agent and at least one medium-chain saturated or unsaturated fatty acid having carbon chain length of C-1 to C-14 or ester thereof, optionally along with excipient(s), giving rise to a synergistic antifungal activity.

### FIELD OF THE INVENTION

The present invention relates to the field of antimicrobials and pharmaceutical sciences. The invention provides antifungal compositions for the management of fungal growth and treatment of fungal infections, including treatment of resistant fungal infections. The present compositions comprise an antifungal agent and a medium-chain saturated or unsaturated fatty acid or ester thereof, optionally along with excipient(s), giving rise to a synergistic antifungal activity.

### BACKGROUND OF THE INVENTION

Fungal infections of the skin are also known as ‘mycoses’. They are common and generally mild. In sick or otherwise immune-suppressed individuals, however, fungi can sometimes cause serious disease. Fungal infections in humans range from superficial, i.e., skin surface to deeply invasive type or disseminated infection.

In general, superficial fungal infections (also known as cutaneous mycosis) can affect the outer layers of skin, nails and hair. The main groups of fungi causing superficial fungal infections are dermatophytes (Trichophyton spp.), yeasts, e.g., Candida, Malassezia, piedra, etc, and moulds. The fungal infections include dermatophytoses, cutaneous candidiasis, dandruff/seborrheic dermatitis (D/SD), onychomysis, intertrigo, and those in psoriasis, atopic dermatitis amongst others.

Dermatophytes are one of the most common filamentous fungal species infecting regions rich in keratin, such as the hair, skin, and nails. They generally grow as branched hyphae inside the layers of stratum corneum. (Weitzman and Summerbell 1995, Clin. Microbiol Rev. 8: 240; Hainer 2003, Am Fam Physician 67: 101). Dermatophytoses, also known as tinea, are rampant among the human population. Tinea can occur at various parts of the body and defined accordingly: tinea capitis (head), tinea corporis and tinea cruris (trunk and groin), tinea pedis (foot), tinea unguium or onychomycosis (nail) etc. Trichophyton rubrum, Trichophyton mentagrophvytes, Trichophyton verrucosum, Microsporum canis, Microsporum gypseum and Epidermophyton floccosum are the major pathogens responsible for dermatophytoses (Weitzman and Summerbell 1995, Clin Microbiol Rev 8: 240; White et a 2008, Eukaryot Cell 7:1238).

Dermatophytic infections manifest as skin lesions which are usually round, erythematous, and itchy due to the inflammatory response triggered by the fungus and its metabolites (Hube et al 2015, J Mycol Med 25:e44). The infection can be mild to severe, depending on the host immune response.

Onychomycosis refers to any fungal infection of the nail where the causative factor can be dermatophytes, yeast or non-dermatophyte moulds. Most toe-nail infections are caused by T. rubrum and T. interdigitale, while yeasts (Candida albicans) are the mostly associated with fingernail infections (Eldridge et al 2014, Expert Rev Anti Infect Ther 12: 1389). In onychomycosis, nails become thicker and separated from the nail bed; white spots and dystrophy may also occur (Trepanier E F and Amsden 1998, Ann Pharmacother 32: 204). Treatment of onychomycosis is a serious challenge due to poor drug penetrability and hence high recurrence rates.

Although dermatophyte infections are restricted to areas of the epidermis, they can be invasive and cause serious widespread infections in immunocompromised patients, with the development of granulomas (Peres et al 2010, An Bras Dermatol 85: 657).

Cutaneous candidiasis is an infection caused by yeasts of the genus Candida. Infections mainly in the mucous membranes of the skin folds are most rampant due to moist conditions. The spectrum of cutaneous candidiasis includes diaper rash, interdigital candidiasis, candida folliculitis, otomycosis, onychia and paronychia. Candida skin infections are mostly associated with erythema, cracking, or maceration (Hay R J 1992, Arch Dis Child 67: 1065; Zuber and Baddam 2001, Postgrad Med 109: 117). Candida albicans has been regarded as the most common causative agent. Host factors (eg, wearing of occlusive clothing, obesity) or disorders affecting the immune system may increase susceptibility to candidal infection.

Candida spp. is responsible for systemic infections in various parts of the human body, including oral cavity, vaginal mucosa, bloodstream and internal organs (Kauffman 2006, Proc Am Thorac Soc 3: 35). Candida albicans, Candida glabrata, Candida parapsilosis, Candida tropicalis, and Candida krusei can cause superficial infections of the oral and vaginal mucosa as well as disseminated bloodstream and deep-tissue infections. Most Candida spp. produce virulence factors including protease factors and the ability of these yeast forms to adhere to the underlying epithelium is an important step in the production of hyphae and tissue penetration.

Candida albicans can also cause deep invasive disease, associated with surgically implanted devices including indwelling intravenous catheters, orthopaedic devices, urinary catheters, intrauterine devices, dialysis vascular grafts and central nervous system implants (Inabo 2006, Scientific Research and Essay 1: 008). Candida infections associated with these diseases generally form biofilms by adhering to the surface of implants. Biofilms of Candida albicans, Candida parapsilosis, Candida glabrata and Candida tropicalis are associated with high indices of hospital morbidity and mortality.

Seborrheic dermatitis is a common, chronic, superficial skin disorder causing scaly, itchy, red skin on the scalp, eyebrows, nasolabial creases, lips, ears, sternal area, axillae, submammary folds, umbilicus, groins, and gluteul crease. The disease is characterized by many shapes, sizes, and surface textures and is often crust-like, yellowish, and accompanied by itching. Seborrheic dermatitis is one of the leading causes of stubborn dandruff and occurs in all age groups. This condition primarily affects the sebaceous cysts present in the skin.

Currently, fungi of the genus Malassezia are believed to be the most likely responsible agents for causing dandruff (Dawson T. L. J. Investig. Dermatol. Symp. Proc. (2007), 12:1519). Most cases of seborrhoeic dermatitis likely involve an inflammatory reaction to the proliferation of the yeast Malassezia. These fungi are highly dependent on external lipids for in vitro growth (Chen T. A, and Hill P. V., Vet Dermatol, (2005), 16:4). Further, the inability to synthesize fatty acids may be complimented by the presence of multiple secreted lipases to aid in utilizing host lipids. Consequently, these fungi metabolize triglycerides present in sebum through these lipases resulting in lipid by-products. Penetration of the top layer of the epidermis, the stratum corneum, by these lipid by-products results in an inflammatory response in susceptible persons, which disturbs homeostasis causing erratic cleavage of stratum corneum cells, further leading to dandruff and seborrheic dermatitis.

There are five major classes of antifungal drugs available to treat fungal infections. They include azoles, allylamines, polyenes, pyrimidine analogs, and echinocandins. (Sanglard and Odds 2002, Lancet Infect Dis 2: 73) Azoles and allylamines which typically inhibit ergosterol synthesis and disrupt fungal growth, and several other classes of antifungals are commonly the mainstay for treatment of superficial fungal infections. The azole antifungals are the most frequent class used to treat Candida infections. Polyenes, such as amphotericin B (AmB), have the ability to bind ergosterol and to destabilize membrane functions (Sanglard 2016, Front Med (Lausanne) 3: 11). Pyrimidine analogs, such as 5-fluorocytosine (5-FC), are metabolized by fungal cells and then destabilized its nucleic acids (RNA, DNA) and therefore result in growth arrest (Sanglard D., 2016, Front Med (Lausanne) 3: 11). Echinocandins like caspofungin, micofungin block the catalytic subunit of the β-1,3 glucan synthase and thus inhibit cell wall biosynthesis (Sanglard D., 2016, Front Med (Lausanne) 3: 11).

Some other mechanisms of action of current drugs are chelation of bivalent cations (ciclopirox), inhibition of leucyl-tRNA synthetase (tavaborole), and interaction with microtubules (griseofulvin) (Subissi et al 2010, Drugs 70: 2133; Gupta et al 2017, Mycopathologia 182: 127). Zinc pyrithione mostly used for the treatment of seborrhoeic dermatitis has fungistatic activity by inhibiting the division of fungal cells. Piroctone olamine exerts its antimycotic action by inhibiting energy metabolism in mitochondria of pathogenic fungi (Dupont et al 2002, Arch Surg 137: 1341).

Both topical agents or oral antifungals are prescribed based on the severity of the fungal infection. Treatment of onychomycosis includes antifungal nail lacquer of ciclopirox or amorolfine. Amorolphine, a morpholine antifungal also depletes ergosterol. Topical efinaconazole was recently approved for the treatment of onychomycosis and after 4 weeks of therapy, cure has been observed in 15-18% of patients (Elewski et al 2013, J Am Acad Dermatol 68: 600). To reduce the long term topical therapy, oral terbinafine is typically recommended as first-line therapy.

The most common treatment of Malassezia infections is the topical application of antifungal agents that reduce the level of the fungus on the scalp. Maintaining the scalp clean is mandatory for sufferers of seborrheic dermatitis. Use of effective anti-dandruff shampoos is, therefore, a significant way of preventing this condition. Typically, the antifungal agent is applied to the scalp as a component of a shampoo or other hair care composition. The disadvantage of such shampoo formulations is that during normal usage the formulation does not remain on the scalp for a period of time sufficient to allow the antifungal agent to achieve its maximal therapeutic effect (Ralph M. Trüeb, JDDG, (2007), 5:356). These are designed to be applied, for example, in the shower or bath, and shortly thereafter rinsed off with water. Typically, the application instructions for such shampoos suggest that the formulation be removed after 3-5 minutes.

One of the antifungal agents, ketoconazole is among the most potent and widely used in anti-dandruff shampoos. However, the exposure time of shampoo is less, due to which the efficacy is poor and relapse rates are higher.

Extensive use of the anti-fungal medication has led to development of anti-fungal resistance. Resistance have been reported effectively from all major pathogenic fungi such as Candida, Aspergillus. Even the dermatophytes, pathogenic fungi (Microsporum spp. Trichophyton spp. etc) specialized in the infection of skin, reported to have shown resistance against existing antifungal molecules (Sanglard 2016, Front Med (Lausanne) 3: 11). Azole drug resistance is most common among dermatophytes as well as Candida, Cryptococcus, and Aspergillus spp., under conditions where the drugs are used regularly, sporadically, and at varying strengths (Pfaller 2012. Am J Med 125(1 Suppl): S3-13). An important issue concerning drug resistance is the occurrence of cross resistance. It has been observed that some Candida isolates with high minimum inhibition concentrations (MICs) to fluconazole have high MICs to itraconazole, although in some strains they are fully sensitive in vitro to the latter (Johnson et al., J Antimicrob Chemother 1995; 36:787-93).

Antifungal resistance can be measured in vitro by drug exposure to the fungal cell as well. Following the protocols of CLSI, Clinical Laboratory Standards Institute; USA or EUCAST, European Committee on Antimicrobial Susceptibility Testing antifungal resistance can be measured in the laboratory (Pfaller et al 2014, Diagn Microbiol Infect Dis 79: 198). Following these protocols, MIC values (given in microgram per milliliter) of individual antifungal compounds against individual isolates of fungus are being established. If MIC of certain isolates are significantly higher than a wild type population, then they are suspected of resistant type. It helps clinicians to decide the future course of treatment with alternative agents.

The majority of resistance mechanisms against these antifungals have also been elucidated at the molecular level in these pathogens. In principle, these mechanisms fall into three distinct categories, including (A) decrease of effective drug concentration within the fungal cell (using drug efflux pumps for instance, through greater expression of efflux pumps such as ATP binding cassette transporters or by getting embedded into biofilm which are highly resistant to antifungal therapy and have better ability to withstand host immune defenses), (B) alteration of drug target (either by over expressing the target molecules or reducing affinity of target by changes in gene and protein levels), and (C) metabolic bypasses (it occurs when given metabolic pathways are perturbed by loss or strong decrease of specific functions). However, in certain cases, clinical non-responders do not harbor microbiologically drug resistant strains and yet fail to respond to antifungal therapy.

Resistance to terbinafine in T. rubrum clinical isolates was shown to be due to single point mutations in the gene encoding for squalene epoxidase (Yamada et al 2017 Antimicrob Agents Chemother pii: AAC.00115-17). Terbinafine resistance in mutants of the Aspergillus species was also reported, and this indicated that there was a mutation in the gene encoding squalene epoxidase (ErgA), resulting in high resistance to this antifungal (Rocha et al 2006, Antimicrob Agents Chemother 50: 2533).

Emerging paradigm of resistance through multiple mechanisms make the therapeutic choices very limited for clinicians for the control of fungal diseases. Simultaneous resistance to different antifungal classes (multidrug resistance) have reduced the efficacy of the already available antifungal agents thereby necessitating the need for developing novel treatment strategies. In absence of newer drugs targeting novel targets, new ways of combining several drugs or potentiating the older drugs are being attempted.

In vitro antifungal combinations are usually assessed on the basis of the fractional inhibitory concentration (FIC) index, which represents the sum of the FICs of each drug tested, where the FIC is determined for each drug by dividing the MIC of each drug when used in combination by the MIC of each drug when used alone. This theory is based on the hypothesis that a drug cannot interact with itself and therefore the effect of a self-drug combination will always be additive, with an FIC index of 1. An FIC index lower or higher than 1 indicates synergy or antagonism, respectively, because less or more drug would be required in order to produce the same effect as the drugs alone (Berenbaum 1989, Pharmacol Rev 41: 93).

Accordingly, there remains a need for a carefully designed antifungal compositions that provide improved antifungal efficacy by a new approach. In the present invention, said objective is achieved.

U.S. Patent Application 2010/0016271 discloses hair conditioning compositions comprising cationic surfactant, triglyceride oil and an anti-dandruff agent. These compositions contain triglyceride oil, which are fatty acid esters of glycerol, and hence act as nutrients and aid in the growth of the fungus. These compositions contain fatty acid material up to 10% having carbon chains from 8 to 30 carbon atoms.

U.S. Pat. No. 5,624,666 describes shampoo compositions containing anionic surfactants, cationic polymers, and zinc pyrithione as an anti-dandruff agent. It describes that conditioning agents such as silicone fluids can optionally be incorporated into the compositions therein. Head & Shoulders® Dandruff Shampoo Plus Conditioner is an example of a marketed product which provides both anti-dandruff and conditioning benefits upon application of the shampoo to hair. However, relapse rates by the use of said products are higher.

The present invention provides improved antifungal compositions which are targeted to overcome drawbacks associated with these prior arts and others that are generally available in the art.

### OBJECTIVE OF THE INVENTION

The primary objective of the invention is to provide improved/synergistic antifungal compositions. Said antifungal compositions comprise an antifungal agent, a fatty acid (C-1 to C14) and/or esters thereof, optionally along with excipients or additives. Yet another objective of the invention is to provide antifungal compositions devoid of more than C-14 fatty acids or their esters for the treatment of topical fungal infections or management of fungal growth. Still another objective of the invention is to provide synergistic compositions for the treatment of fungal infections or management of fungal growth, against both resistant and non-resistant fungi.

### BRIEF DESCRIPTION OF THE DRAWINGS

FIG. 1A is a schematic representation of the checkerboard assay to test synergistic, additive or antagonistic effects of two test agents. FIG. 1B is a representative data set for checkerboard assay of clotrimazole and oleic acid in C. albicans. FIG. 1C is a graphical representation of the decrease in potency of clotrimazole with increasing concentrations of oleic acid.

FIG. 2 is a photograph showing the effect of nutrients/fatty acids or their esters provided in the culture media on the growth of M. furfur (MTCC 1374).

FIG. 3 is a graphical representation showing that propylene glycol monocaprylate in a formulation with an allylamine is active/synergistic against terbinafine resistant Trichophyton interdigitale (GTB-2S) in an in vitro time kill assay.

FIG. 4 shows comparison of exemplary compositions of the invention comprising clotrimazole formulations vs marketed clotrimazole formulations in an azole resistant T. rubrum strain (GTB-3FR-TS).

FIG. 5 shows comparison of exemplary compositions of the invention comprising clotrimazole vs marketed clotrimazole formulations in an azole resistant C. albicans strain (MTCC-227).

FIG. 6 is a graphical representation showing that propylene glycol monocaprylate in an exemplary formulation with an azole (luliconazole) is active/synergistic against azole resistant C. albicans (MTCC-227) in an in vitro time kill assay.

FIG. 7 shows comparison of exemplary compositions of the invention comprising comprising propylene glycone monocaprylate and luliconazole vs marketed luliconazole formulations in an azole resistant C. albicans strain (MTCC-227).

FIG. 8 is a graphical representation showing that exemplary composition of the invention comprising clotrimazole and propylene glycol monocaprylate has higher/synergistic efficacy in a cutaneous candidiasis animal model using azole resistant C. albicans (MTCC 227).

FIG. 9 shows that an exemplary composition of the invention comprising luliconazole and with propylene glycol monocaprylate has higher/synergistic efficacy in a murine tinea model using a pathogenic Trichophyton mentagrophyte strain (ATCC 24953).

FIG. 10 is a graphical representation showing enhanced efficacy of exemplary shampoo formulation containing ketoconazole (2%) and ester derivatives of caprylic acid compared to marketed ketoconazole shampoo formulations in an in vitro time kill assay against M. furfur (MTCC 1374).

### BRIEF DESCRIPTION OF THE TABLES

Table 1: Representative data from checkerboard assays of combination of caprylic acid (C8) with antifungals (various classes) on Trichophyton rubrum (ATCC 28188).

Table 2: Representative data from checkerboard assays of combination of propylene glycol monocaprylate (caprylic acid ester) with antiftingals (various classes) on Trichophyton rubrum (ATCC 28188).

Table 3: Representative data from checkerboard assays of combination of glyceryl monocaprylate with antifungals (various classes) on Trichophyton rubrum (ATCC 28188).

Table 4: Representative data from checkerboard assays of combination of undecylinic acid (C11) with antifungals (various classes) on Trichophyton rubrum (ATCC 28188).

Table 5: Representative data from checkerboard assays of combination of lauric acid (C12) with antifungals (various classes) on Trichophyton rubrum (ATCC 28188).

Table 6: Representative data from checkerboard assays of combination of propylene glycol monolaurate (lauric acid ester C12 fatty acid) with antifungals (various classes) on Trichophyton rubrum (ATCC 28188).

Table 7: Representative data showing synergistic action of terbinafine or butenafine with caprylic acid (C8), or its esters (propylene glycol monocaprylate and glyceryl monocaprylate) against terbinafine resistant Trichophyton interdigitale (GTB-2S).

Table 8: Representative data from checkerboard assays of combinations of luliconazole with caprylic acid (C8) or glyceryl monocaprylate on C. albicans (ATCC-90028).

Table 9: Representative data from checkerboard assays of combination of caprylic acid (C8) with variousantifungals on azole resistant C. albicans MTCC 227.

Table 10: Representative data from checkerboard assays of combination of propylene glycol monocaprylate (C8 ester) with various antifungals on azole resistant C. albicans MTCC 227.

Table 11: Representative data from checkerboard assays of combination of glyceryl monocaprylate (C8 ester) with various antifungals on azole resistant C. albicans MTCC 227.

Table 12: Representative data from checkerboard assays of combination of undecylenic acid (C11) with various antifungals on azole resistant C. albicans MTCC 227.

Table 13: Representative data from checkerboard assays of combination of lauric acid (C12) with various antifungals on azole resistant C. albicans MTCC 227.

Table 14: Representative data from checkerboard assays of combination of propylene glycol monolaurate (ester of C12 fatty acid) with various antifungals on azole resistant C. albicans MTCC 227.

Table 15: Piroctone olamine based oil compositions containing Caprylic acid (C8 fatty acid).

Table 16: Ketoconazole based oil compositions containing Caprylic acid (C8 fatty acid).

Table 17: Results of minimum inhibitory concentration (MIC) for oil compositions of piroctone olamine and caprylic acid.

Table 18: Results of MIC for oil compositions of ketoconazole and caprylic acid against M. furfur (MTCC 1374).

Table 19: Oil compositions containing piroctone olamine as antifungal agent and caprylic acid and/or its ester.

Table 20: MIC of oil compositions containing piroctone olamine and caprylic acid and/or its ester against M. furfur (MTCC 1374).

Table 21: MIC for oil compositions containing piroctone olamine and caprylic acid and/or its ester against M. obtusa (CBS 7876).

Table 22: Oil compositions containing ketoconazole as antifungal agent and caprylic acid and/or its ester.

Table 23: Oil compositions containing piroctone olamine and ketoconazole in combination with caprylic acid and/or its ester.

Table 24: Oil compositions containing piroctone olamine as antifungal agent, Minoxidil and caprylic acid and/or its ester.

Table 25: Gel compositions containing antifungal agents devoid of C-15 or greater fatty acids/esters.

Table 26: Zone of inhibition of gel compositions containing piroctone olamine and caprylic acid and/or its ester against M. furfur.

Table 27: Preparation of cream compositions containing antifungal agents piroctone olamine or ketoconazole and caprylic acid ester derivative.

Table 28: Exemplary clotrimazole (1%) topical cream formulations with at least one or two medium chain fatty acids (C-1 to C-14) and derivatives thereof.

Table 29: Exemplary luliconazole (1%) topical cream formulations with at least one or two medium chain fatty acids (C-1 to C-14) and derivatives thereof.

Table 30: Exemplary topical cream formulations containing 1% terbinafine with at least one or two medium chain fatty acids (C-1 to C-14) and derivatives thereof.

Table 31: Exemplary luliconazole (1%) topical lotion formulations without ethanol and containing at least one or two medium chain fatty acids (C-1 to C-14) and derivatives thereof.

Table 32: Exemplary luliconazole (1%) topical lotion formulations with ethanol and containing at least one or two medium chain fatty acids (C-1 to C-14) and derivatives thereof.

Table 33: Exemplary topical nail solutions containing 1% efinaconazole with at least one or two medium chain fatty acids (C-1 to C-14) and derivatives thereof.

Table 34: Exemplary topical shampoo formulations containing ketoconazole or combination of ketoconazole-zinc pyrithione (ZPTO) with structured surfactants containing at least one or two medium chain fatty acids (C-1 to C-14) and derivatives thereof.

Table 35: Exemplary topical shampoo formulations containing ketoconazole with mild sulphate free surfactants containing at least one or two medium chain fatty acids (C-1 to C-14) and derivatives thereof.

Table 36: Exemplary hair serum formulations containing an antifungal agent with at least one or two medium chain fatty acids (C-1 to C-14) and derivatives thereof.

Table 37: Exemplary body lotion formulations containing an antifungal agent with at least one or two medium chain fatty acids (C-1 to C-14) and derivatives thereof.

Table 38: Exemplary formulations for coating surgical implants containing an antimicrobial/antifungal agent with at least one or two medium chain fatty acids (C-1 to C-14) and derivatives thereof.

### SUMMARY OF THE INVENTION

The present invention showcases that specific fatty acids, especially medium chain fatty acids (C1 to C14) and esters thereof demonstrate synergistic antifungal activity in combination with various antifungal agents. Moreover, the invention proves that fatty acids and/or esters in combination with various antifungal agents demonstrate synergistic antifungal activity against drug-resistant fungi in addition to drug-susceptible fungi.

Accordingly, in one aspect, the present invention provides antifungal compositions that comprise at least an antifungal agent and a fatty acid (C1 to C14) or ester thereof.

In some embodiments, the antifungal composition of the invention further comprises at least one excipient.

In exemplary embodiments, the antifungal composition of the invention is devoid of C-15 or greater fatty acids or their esters as these long chain fatty acids/esters serve as nutrients for the growth of the fungus. In other words, the present invention shows that such long chain fatty acids/esters help in enhancing the fungal growth rather than growth inhibition.

In some embodiments, the antifungal composition comprises an antifungal agent, a fatty acid with less than C-15 chain length or its ester (synergistic to the antifungal agent), and at least one excipient.

In some embodiments, the antifungal composition comprises an antifungal agent, a fatty acid (C1 to C14) or its ester thereof (synergistic to the antifungal agent), and at least one excipient.

In some exemplary embodiments, the synergistic antifungal composition of the invention comprises an antifungal agent, a fatty acid (C11 to C14) or its ester thereof (synergistic to the antifungal agent), and at least one excipient.

In other embodiments, the synergistic antifungal composition comprises an antifungal agent, a fatty acid (C1 to C10) or its ester thereof (synergistic to the antifungal agent), and at least one excipient.

In some exemplary embodiments, the synergistic antifungal composition of the invention comprises an antifungal agent, a fatty acid having carbon chain length of C8 or its ester thereof, and at least one excipient.

The present invention provides antifungal compositions formulated for topical, local or systemic delivery for management of fungal growth including growth caused by drug-resistant fungi.

The present invention provides antifungal compositions formulated for topical, local or systemic delivery for treatment of skin based fungal infections including infections caused by drug-resistant fungi.

The present invention further provides antifungal creams or lotions formulated for topical, local or systemic delivery for treatment of tinea infections or prevent or reduce relapse of tinea infections.

The present invention further provides antifungal creams or lotions formulated for topical, local or systemic delivery for treatment of seborrheic dermatitis or prevent or reduce relapse of the same.

The present disclosure provides antifungal cream or lotions formulated for topical, local or systemic delivery for treatment of Candida infections.

The present disclosure also provides antifungal solutions/compositions for treating or managing nail infections. The present invention further provides topical antifungal oil compositions that eliminate existing dandruff on the scalp, or prevent or reduce relapse of dandruff formation.

The present invention further provides antifungal shampoo formulations formulated for topical, local or systemic delivery for treatment of fungal infections of the scalp (like dandruff, tinea capitis).

The present disclosure further describes antifungal compositions that can be used for the treatment of surgical implant associated fungal infections including drug-resistant or drug-susceptible fungal infections.

The disclosure also provides use of present antifungal compositions for management of fungal growth or treatment of fungal infections as described herein.

The present disclosure further provides methods of preparing antifungal compositions described herein.

### DETAILED DESCRIPTION OF THE INVENTION

The present disclosure relates to an antifungal composition comprising at least one antifungal agent, at least one fatty acid or ester thereof, and optionally one or more excipient, wherein the fatty acid has a carbon chain length ranging from C-1 to C-14, and wherein the composition has synergistic antifungal activity.

In an embodiment of the present disclosure, the fatty acid or ester thereof in the antifungal composition is a saturated or unsaturated fatty acid or ester of said saturated or unsaturated fatty acid.

In another embodiment of the present disclosure, the fatty acid in the antifungal composition has a carbon chain length ranging from C-11 to C-14.

In yet another embodiment of the present disclosure, the fatty acid in the antifungal composition has a carbon chain length ranging from C-1 to C-10.

In still another embodiment of the present disclosure, the fatty acid in the antifungal composition is selected from the group consisting of formic acid (C1), acetic acid (C2), propionic acid (C3), butyric acid (C4), valeric acid (C5), caproic acid (C6), enanthic acid (C7), caprylic acid (C8), pelargonic acid (C9), capric acid (C10), undecylic acid (C11), lauric acid (C12), tridecylic acid (C13), myristic acid (C14) and corresponding unsaturated fatty acids thereof.

In still another embodiment of the present disclosure, the fatty acid in the antifungal composition is undecylic acid (C11), lauric acid (C12), tridecylic acid (C13), myristic acid (C14) or corresponding unsaturated fatty acids thereof.

In still another embodiment of the present disclosure, the fatty acid in the antifungal composition is formic acid (C1), acetic acid (C2), propionic acid (C3), butyric acid (C4), valeric acid (C5), caproic acid (C6), enanthic acid (C7), caprylic acid (C8), pelargonic acid (C9), capric acid (C10) or corresponding unsaturated fatty acids thereof.

In still another embodiment of the present disclosure, the fatty acid ester in the antifungal composition is selected from the group consisting of ester of formic acid (C1), ester of acetic acid (C2), ester of propionic acid (C3), ester of butyric acid (C4), ester of valeric acid (C5), ester of caproic acid (C6), ester of enanthic acid (C7), ester of caprylic acid (C8), ester of pelargonic acid (C9), ester of capric acid (C10), ester of undecylic acid (C11), ester of lauric acid (C12), ester of tridecylic acid (C13), ester of myristic acid (C14) and esters of corresponding unsaturated fatty acids thereof.

In still another embodiment of the present disclosure, the fatty acid ester in the antifungal composition is selected from the group consisting of propylene glycol monocaprylate, propylene glycol monolaurate, propylene glycol monocaprate, glyceryl monocaprylate, glyceryl monolaurate, glyceryl monocaprate, glyceryl dicaprylate, glyceryl dilaurate, glyceryl dicaprate, glyceryl mono-di caprate, glyceryl mono-di caprylate, glyceryl mono-di laurate, triglycerides of caprylic acid, capric acid, lauric acid and their mixtures, and combinations thereof.

In still another embodiment of the present disclosure, the fatty acid ester in the antifungal composition is propylene glycol monocaprylate, propylene glycol monolaurate, glycerol monocaprylate, glycerol monolaurate, or any combination thereof.

In still another embodiment of the present disclosure, the antifungal agent of the antifungal composition is selected from the group consisting of allylamines, benzylamines, azoles, polyenes, echinocandins, N-hydroxy pyridone, N-hydroxy pyrithione or metal coordination complexes, tavaborole, flucytosine, griseofulvin, hinokitol and combinations thereof.

In still another embodiment of the present disclosure, the N-hydroxy pyridone is piroctone olamine, ciclopirox olamine or a combination thereof; the N-hydroxy pyrithione or the metal coordination complex is zinc pyrithione or any respective bivalent metal coordinating complexes or combinations thereof; allylamines are selected from the group consisting of terbinafine, amorolfine, naftifine and combinations thereof; the benzylamine is butenafine; the azoles are imidazoles, triazoles or thiazoles selected from the group consisting of ketoconazole, climbazole, miconazole nitrate, fluconazole, econazole, saperconazole, oxiconazole, clotrimazole, bifonazole, butoconazole, fenticonazole, isoconazole, omoconazole, sertaconazole, sulconazole, tioconazole, luliconazole, chlormidazole, croconazole, eberconazole, omoconazole, isoconazole, neticonazole, albaconazole, efinaconazole, fosfluconazole, epoxiconazole, fluconazole, isavuconazole, itraconazole, posaconazole, propiconazole, ravuconazole, terconazole, voriconazole, hexaconazole, abafungin and combinations thereof; the polyenes are selected from the group consisting of amphotericin B, natamycin, nystatin and combinations thereof; and the echinocandins are selected from the group consisting of caspofungin, anidulafungin, micafungin and combinations thereof.

In still another embodiment of the present disclosure, the antifungal agent in the antifungal composition is selected from the group consisting of piroctone olamine, zinc pyrithione, ketoconazole, clotrimazole, luliconazole, terbinafine, efinaconazole, bifonazole, amphotericin B, caspofungin, ciclopirox olamine, climbazole, miconazole nitrate, itraconazole, fluconazole, econazole, terconazole, saperconazole, amorolfine, oxiconazole, butenafine, nafiifine and combinations thereof.

In still another embodiment of the present disclosure, the excipient in the antifungal composition is selected from the group consisting of additive, solvent, oil, emulsifier, surfactant, stabilizer, cooling agent, preservative, antioxidant, gelling agent, moisturizing agent, emollient, penetration enhancer, colorant, fragrance, pH modifiers, conditioning agent, pearlizing agents, skin barrier repair agents, and combinations thereof.

In still another embodiment of the present disclosure, the antifungal composition comprises about 0.01% to 20% by weight of the antifungal agent.

In still another embodiment of the present disclosure, the antifungal composition comprises about 0.01% to 15% by weight of the antifungal agent.

In still another embodiment of the present disclosure, the antifungal composition comprises about 0.01% to 30% by weight of the saturated or unsaturated fatty acid or ester thereof.

In still another embodiment of the present disclosure, the antifungal composition comprises about 0.01% to 20% by weight of the saturated or unsaturated fatty acid or ester thereof.

In still another embodiment of the present disclosure, the antifungal composition comprises about 45% to 99% by weight of the excipient.

In still another embodiment of the present disclosure, the antifungal composition comprises about 80% to 99% by weight of the excipient.

In still another embodiment of the present disclosure, the antifungal composition comprises saturated or unsaturated caprylic acid or an ester thereof and antifungal agent selected from the group consisting of allylamines, benzylamines, azoles, polyenes, echinocandins, N-hydroxy pyridones, N-hydroxy pyrithiones and combinations thereof, and optionally at least one excipient.

In still another embodiment of the present disclosure, the antifungal composition comprises saturated or unsaturated caprylic acid or an ester thereof and antifungal agent selected from the group consisting of terbinafine, butenafine, clotrimazole, ketoconazole, luliconazole, bifonazole, efinaconazole, amphotericin B, caspofungin, zinc pyrithione, piroctone olamine and combinations thereof, and optionally at least one excipient.

In still another embodiment of the present disclosure, the antifungal composition comprises propylene glycol monocaprylate and antifungal agent selected from the group consisting of allylamines, benzylamines, azoles, polyenes, echinocandins, N-hydroxy pyridones, N-hydroxy pyrithiones and combinations thereof, and optionally at least one excipient.

In still another embodiment of the present disclosure, the antifungal composition comprises propylene glycol monocaprylate and antifungal agent selected from the group consisting of terbinafine, butenafine, clotrimazole, ketoconazole, luliconazole, bifonazole, efinaconazole, amphotericin B, caspofungin, zinc pyrithione, piroctone olamine and combinations thereof, and optionally at least one excipient.

In still another embodiment of the present disclosure, the antifungal composition comprises saturated or unsaturated lauric acid or propylene glycol monolaurate, and antifungal agent selected from the group consisting of allylamines, benzylamines, azoles, polyenes, echinocandins, N-hydroxy pyridones, N-hydroxy pyrithiones and combinations thereof, and optionally at least one excipient.

In still another embodiment of the present disclosure, the antifungal composition comprises saturated or unsaturated lauric acid or propylene glycol monolaurate and antifungal agent selected from the group consisting of terbinafine, butenafine, clotrimazole, ketoconazole, luliconazole, bifonazole, efinaconazole, amphotericin B, caspofungin, zinc pyrithione, piroctone olamine and combinations thereof, and optionally at least one excipient.

In still another embodiment of the present disclosure, the antifungal composition comprises fatty acid ester selected from the group consisting of glyceryl monocaprylate, glyceryl monolaurate, glyceryl monocaprate, glyceryl dicaprylate, glyceryl dilaurate, glyceryl dicaprate, glyceryl mono-di caprate, glyceryl mono-di caprylate and glyceryl mono-di laurate, triglycerides of caprylic acid, capric acid, lauric acid and their mixtures, and antifungal agent selected from the group consisting of allylamines, benzylamines, azoles, polyenes, echinocandins, N-hydroxy pyridones, N-hydroxy pyrithiones and combinations thereof, and optionally at least one excipient.

In still another embodiment of the present disclosure, the antifungal composition comprises fatty acid ester selected from the group consisting of glyceryl monocaprylate, glyceryl monolaurate, glyceryl dicaprylate, glyceryl dilaurate, glyceryl monocaprate, glyceryl dicaprate, glyceryl mono-di caprate, glyceryl mono-di caprylate and glyceryl mono-di laurate, triglycerides of caprylic acid, capric acid, lauric acid and their mixtures, and antifungal agent selected from the group consisting of terbinafine, butenafine, clotrimazole, ketoconazole, luliconazole, bifonazole, efinaconazole, amphotericin B, caspofungin, zinc pyrithione, piroctone olamine and combinations thereof, and optionally at least one excipient.

In still another embodiment of the present disclosure, the antifungal composition comprises saturated or unsaturated capric acid or an ester thereof and antifungal agent selected from the group consisting of allylamines, benzylamines, azoles, polyenes, echinocandins, N-hydroxy pyridones, N-hydroxy pyrithiones and combinations thereof, and optionally at least one excipient.

In still another embodiment of the present disclosure, the antifungal composition comprises saturated or unsaturated capric acid or an ester thereof and antifungal agent selected from the group consisting of terbinafine, butenafine, clotrimazole, ketoconazole, luliconazole, bifonazole, efinaconazole, amphotericin B, caspofungin, zinc pyrithione, piroctone olamine and combinations thereof, and optionally at least one excipient.

In still another embodiment of the present disclosure, the antifungal composition comprises saturated or unsaturated undecylic acid or an ester thereof and antifungal agent selected from the group consisting of allylamines, benzylamines, azoles, polyenes, echinocandins, N-hydroxy pyridones, N-hydroxy pyrithiones and combinations thereof, and optionally at least one excipient.

In still another embodiment of the present disclosure, the antifungal composition comprises saturated or unsaturated undecylic acid or an ester thereof and antifungal agent selected from the group consisting of terbinafine, butenafine, clotrimazole, ketoconazole, luliconazole, bifonazole, efinaconazole, amphotericin B, caspofungin, zinc pyrithione, piroctone olamine and combinations thereof, and optionally at least one excipient.

In still another embodiment of the present disclosure, the antifungal composition comprises (a) piroctone olamine, propylene glycol monocaprylate and at least one excipient, (b) zinc pyrithione, propylene glycol monocaprylate and at least one excipient, (c) ketoconazole, propylene glycol monocaprylate and at least one excipient, (d) ketoconazole, zinc pyrithione, propylene glycol monocaprylate and at least one excipient, (e) clotrimazole, propylene glycol monocaprylate and at least one excipient, (f) luliconazole, propylene glycol monocaprylate and at least one excipient, (g) terbinafine, propylene glycol monocaprylate and at least one excipient, (h) efinaconazole, propylene glycol monocaprylate and at least one excipient, (i) itraconazole, propylene glycol monocaprylate and at least one excipient, (j) amphotericin B, propylene glycol monocaprylate and at least one excipient, (k) caspofungin, propylene glycol monocaprylate and at least one excipient, (1) ciclopirox olamine, propylene glycol monocaprylate and at least one excipient, (m) butenafine, propylene glycol monocaprylate and at least one excipient, (n) ketoconazole, propylene glycol monocaprylate, glyceryl mono-di caprate/caprylate and at least one excipient, or (o) ketoconazole, zinc pyrithione, propylene glycol monocaprylate, glyceryl mono-di caprate, glyceryl mono-di caprylate and at least one excipient.

In still another embodiment of the present disclosure, the antifungal composition comprises (a) piroctone olamine, glyceryl monocaprylate or glyceryl mono-di caprate or glyceryl mono-di caprylate, and at least one excipient, (b) zinc pyrithione, glyceryl monocaprylate and at least one excipient, (c) ketoconazole, glyceryl monocaprylate and at least one excipient, (d) ketoconazole, zinc pyrithione, glyceryl monocaprylate and at least one excipient, (e) clotrimazole, glyceryl monocaprylate and at least one excipient, (f) luliconazole, glyceryl monocaprylate and at least one excipient, (g) terbinafine, glyceryl monocaprylate and at least one excipient, (h) efinaconazole, glyceryl monocaprylate and at least one excipient, (i) caspofungin, glyceryl monocaprylate and at least one excipient, (j) ciclopirox olamine, glyceryl monocaprylate and at least one excipient, (k) butenafine, glyceryl monocaprylate and at least one excipient, (l) terbinafine, glyceryl monocaprylate and at least one excipient, (m) butenafine, caprylic acid and at least one excipient, (n) terbinafine, caprylic acid and at least one excipient, (o) luliconazole, caprylic acid and at least one excipient, (p) clotrimazole, caprylic acid and at least one excipient, (q) efinaconazole, caprylic acid and at least one excipient, (r) ketoconazole, caprylic acid and at least one excipient, (s) ketoconazole, caprylic acid, propylene glycol monocaprylate and at least one excipient, (t) piroctone olamine, ketoconazole, caprylic acid and at least one excipient, (u) piroctone olamine, ketoconazole, caprylic acid, propylene glycol monocaprylate and at least one excipient, or (v) itraconazole, caprylic acid and at least one excipient.

In still another embodiment of the present disclosure, the antifungal composition comprises (a) piroctone olamine, undecylenic acid and at least one excipient, (b) zinc pyrithione, undecylenic acid and at least one excipient, (c) ketoconazole, undecylenic acid and at least one excipient, (d) ketoconazole, zinc pyrithione, undecylenic acid and at least one excipient, (e) clotrimazole, undecylenic acid and at least one excipient, (f) luliconazole, undecylenic acid and at least one excipient, (g) terbinafine, undecylenic acid and at least one excipient, (h) efinaconazole, undecylenic acid and at least one excipient, (i) itraconazole, undecylenic acid and at least one excipient, (j) amphotericin B, undecylenic acid and at least one excipient, (k) caspofungin, undecylenic acid and at least one excipient, or (l) ciclopirox olamine, undecylenic acid and at least one excipient.

In still another embodiment of the present disclosure, the antifungal composition comprises (a) piroctone olamine, lauric acid or propylene glycol monolaurate and at least one excipient, (b) zinc pyrithione, lauric acid or propylene glycol monolaurate and at least one excipient, (c) ketoconazole, lauric acid or propylene glycol monolaurate and at least one excipient, (d) ketoconazole, zinc pyrithione, lauric acid or propylene glycol monolaurate and at least one excipient, (e) clotrimazole, lauric acid or propylene glycol monolaurate and at least one excipient, (f) luliconazole, lauric acid or propylene glycol monolaurate and at least one excipient, (g) terbinafine, lauric acid or propylene glycol monolaurate and at least one excipient, (h) efinaconazole, lauric acid or propylene glycol monolaurate and at least one excipient, (i) itraconazole, lauric acid or propylene glycol monolaurate and at least one excipient, (j) amphotericin B, lauric acid or propylene glycol monolaurate and at least one excipient, (k) caspofungin, lauric acid or propylene glycol monolaurate and at least one excipient, (l) ciclopirox olamine, lauric acid or propylene glycol monolaurate and at least one excipient, (m) clotrimazole, a fatty acid or ester selected from the group consisting of propylene glycol monolaurate, glycerol monolaurate, propylene glycol monocaprylate, glyceryl monocaprylate, glyceryl mono-di-caprylate/caprate or any combination thereof, and at least one excipient, (n) luliconazole, a fatty acid or ester selected from the group consisting of propylene glycol monocaprylate, propylene glycol monolaurate, glyceryl monocaprylate, glyceryl mono-di-caprylate/caprate or any combination thereof, and at least one excipient, or, (o) terbinafine, a fatty acid or ester selected from the group consisting of propylene glycol monocaprylate, glycerol caprylate/caprate or a combination thereof, and at least one excipient.

In still another embodiment of the present disclosure, the antifungal composition is devoid of C-15 or greater fatty acids, or C-15 or greater fatty acid esters.

In still another embodiment of the present disclosure, the antifungal composition is formulated for topical administration, local administration, systemic administration, or any combination thereof.

In still another embodiment of the present disclosure, the antifungal composition is formulated into cream, oil, lotion, serum, gel, emugel, hydrogel, shampoo, nail varnish, ointment, foam, spray, aerosol, coating for material selected from surgical implants, silicon tube, catheter, valves, stents, or suture; or any combination of formulations thereof.

The present disclosure further relates to a method for treating a fungal infection in a subject in need thereof or managing fungal growth, comprising administering the antifungal composition described herein to the subject, or contacting the antifungal composition described herein with the fungus.

In an embodiment of the present disclosure, the method of treating or managing comprises inhibiting the fungal growth, reducing the fungal growth, eliminating the fungus, curing drug resistant fungal infections, treatment of fungal infections in clinical non-responders and patients with barrier defects, or any combination thereof.

In another embodiment of the present disclosure, the treatment described herein includes medical treatment, cosmetic treatment, or a combination thereof.

In yet another embodiment of the present disclosure, the fractional inhibitory concentration (FIC) index of the compositions described herein is less than 1.

In still another embodiment of the present disclosure, the fungal infection or fungal growth is caused by fungi selected from the group consisting of Malassezia species, Trichophyton species, Microsporum species, Epidermophyton species, Candida species, Aspergillus species, Cryptococcus species and combinations thereof.

In still another embodiment of the present disclosure, the fungal infection or fungal growth is caused by Malassezia spp. selected from the group consisting of M. furfur, M. pachydermatis, M. globosa, M. restricta, M. sloofiae, M. sympodialis, M. nana, M. yamatoensis, M. dermatis, M. obtusa, M. japonica, M. caprae, M. cuniculi, M. equine, and M. arunalokei; Trichophyton spp. selected from the group consisting of T. rubrum, T. mentagrophyte, T. interdigitale, T. tonsurans, T. schoenleinii, T. violaceum, T. abissinicum, T. balcaneum, T. circonvolutum, T. concentricum, T. eboreum, T. errinacei, T. fischeri, T. fluviomuniense, T. glabrum, T. gourvilii, T. kanei, T. kuryangei, T. megninii, T. pedis, T. proliferans, T. raubitschekii, T. redellii, T. rodhainii, T. simii, T. soudanense, T. thuringiense, T. verrucosum, T. violaceum and Trichophyton yaoundei; Candida spp. selected from the group consisting of C. albicans, C. glabrata, C. guilliermondii, C. krusei, C. lusitaniae, C. parapsilosis, C. tropicalis, C. colliculosa, C. dubliniensis, C. famata, C. haemulonii, C. inconspicua, C. intermedia, C. kefyr, C. lipolytica, C. metapsilosis, C. norvegensis, C. orthopsilosis, C. pelliculosa, C. pulcherrima, C. nrugose. C. utilis, C. viswanathii, and C. zeylanoides; Microsporum spp. selected from the group consisting of M. audouinii, M. canis, M. amazonicum, M. boullardii, M. cookie, M, distortum, M. duboisii, M equinum, M. ferrugineum, M. fulvum, M. gallinae, M. gypseum, M. langeronii, M. nanum, M. persicolor, M. praecox, M. ripariae and M. rivalieri; Epidermaphyton spp such as E. floccosum; and other non-dermatophytes including but not limited to Aspergillus spp. selected from the group consisting of A. fumigates, A. flavus, A. nidulans, A. terreus, A. lentulus, A. niger, A. alliaceus, A. arvii, A. brevipes, A. calidoustus, A. conjunctus, A. deflectus, A. duricaulis, A. emericella, A. fischerian, A. fumigatiaffinis, A. fumisvnnematus, A. granulosus, A. novofumigatus, A. panamensis, A. quadrilineatus, A. udagawae, A. unilateralis and A. ustus; and Cryptococcus spp. selected from the group consisting of C. neoformans, C. gattii, C. albidus, C. bacillisporus, C. decagatti, C. deuterogatti, C. laurentii, C. tetragatti and C. uniguttulatus; or any combination of fungi thereof.

In still another embodiment of the present disclosure, the fungus is resistant or susceptible to the antifungal agent comprised in the antifungal composition.

In still another embodiment of the present disclosure, the subject described herein is mammal including human.

The present disclosure also relates to antifungal compositions described herein for use as a medicament.

In an embodiment of the present disclosure, the antifungal compositions described herein are employed for use in treating fungal infection.

The present disclosure further relates to use of antifungal compositions described herein for managing fungus growth.

The present disclosure further relates to a method of preparing the antifungal compositions described herein, comprising either of:
a) mixing at least one antifungal agent, at least one fatty acid or ester thereof, and optionally one or more excipient, in any order; wherein the fatty acid has a carbon chain length ranging from C-1 to C-14; or
b) adding at least one fatty acid or ester thereof to a composition comprising at least one antifungal agent, wherein the fatty acid has a carbon chain length ranging from C-1 to C-14; or
c) replacing C-15 or greater fatty acid or ester thereof with at least one C-1 to C-14 fatty acid or ester thereof, in a composition comprising at least one antifungal agent and said C15 or greater fatty acid or ester thereof,
   to obtain the antifungal composition.

In an embodiment of the present disclosure, the at least one fatty acid or ester thereof being mixed or added or replaced in the above method has a carbon chain length ranging from C-11 to C-14, or C-8 to C-10.

In another embodiment of the above method, the concentration of the at least one antifungal agent is about 0.01% to 20%, concentration of the at least one fatty acid or ester thereof is about 0.01% to 30%, and concentration of the one or more excipient is about 45% to 99%, wherein the fatty acid has a carbon chain length ranging from C-1 to C-14.

While the invention is susceptible to various modifications and alternative forms, specific aspect thereof has been shown by way of various examples and drawings and will be described in detail below. It should be understood, however that it is not intended to limit the invention to the particular forms disclosed, but on the contrary, the invention is to cover all modifications, equivalents, and alternative falling within the spirit and the scope of the invention as defined by the appended claims.

In the following detailed description of the aspects of the invention, references are made to the accompanying drawings and graphs that form part hereof and in which are shown by way of illustration specific aspects in which the invention may be practiced. The aspects are described in sufficient details to enable those skilled in the art to practice the invention, and it is to be understood that other aspects may be utilized and that changes may be made without departing from the scope of the present invention.

The present invention is based in part on inventors' demonstration that medium carbon chain fatty acids and/or esters thereof unexpectedly and surprisingly show synergistic effects in antifungal activity when combined with various antifungal agents. Further, medium chain fatty acids and esters in combination with various antifungal agents also demonstrate synergistic antifungal activity against both drug susceptible and resistant fungi (known to be resistant against the particular antifungal agent).

Accordingly, the present invention is directed to antifungal compositions for the management of fungal growth or treatment of fungal infections, including resistant fungal infections, wherein the composition comprises of: (i) at least one antifungal agent; and (ii) at least one medium carbon chain fatty acid or ester thereof; these two components being synergistic in their antifungal activity.

In all listed embodiments, a fractional inhibitory concentration (FIC) index of the tested combinations of antifungal agents and the medium carbon chain fatty acid or ester thereof is less than 1 establishing the synergy of the present compositions.

In some embodiments, the antifungal composition comprises at least one antifungal agent, at least one fatty acid or ester thereof having carbon chain length ranging from C-1 to C-14, and at least one excipient wherein the antifungal agent and medium chain fatty acid or ester thereof have been shown to be synergistic in their antifungal activity.

In some embodiments, the antifungal composition wherein the antifungal agent and medium chain fatty acid or ester thereof have been shown to be synergistic in their antifungal activity further comprises at least one oil (excipient).

In some embodiments of the present disclosure, methods of preparing the antifungal compositions are provided which includes preparing said compositions by: (i) mixing individual components or their respective solutions in any order viz. at least one antifungal agent, at least one fatty acid or ester thereof having carbon chain length ranging from C-1 to C-14 and optionally at least one excipient; (ii) employing existing/known antifungal based compositions and modifying the same to obtain the present compositions.

Yet another aspect of the present invention is to provide methods for the treatment of fungal infections comprising administering to a subject in need thereof, an antifungal composition of the present invention. In some embodiments, the fungal infection is a resistant fungal infection which is treated by the compositions of the present invention.

As used herein, the term “synergistic” or “synergy” means that the antifungal effect achieved with combinations of antifungal agents and medium carbon chain fatty acids/esters is greater than the sum of the effects that results from using the anti-fungal agent and said fatty acid/ester individually. In the present disclosure, “synergy” is being achieved by the combination of antifungal agents and medium carbon chain fatty acids/esters, a term, which is therefore also applicable to compositions comprising the said combinations, with or without any additional component. Accordingly, the terms “synergistic antifungal composition”, “synergistic combination”, “synergistic antifungal combination” or “synergistic composition” may be used interchangeably in the present disclosure and refer to the compositions/combinations of the disclosure comprising at least one antifungal agent, at least one fatty acid or ester thereof, and with/without excipient(s)/additional agent(s). The present disclosure also similarly provides for the “antifungal composition” of the present disclosure, having an antifungal activity, wherein such antifungal activity is synergistic antifungal activity.

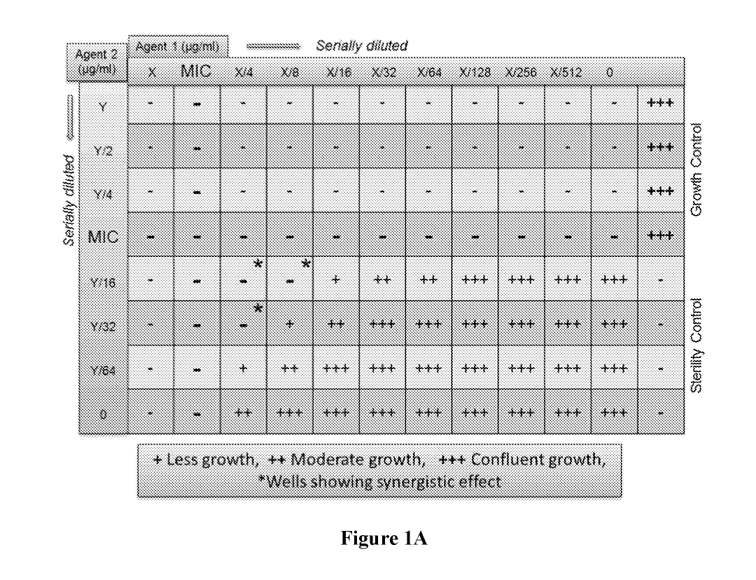

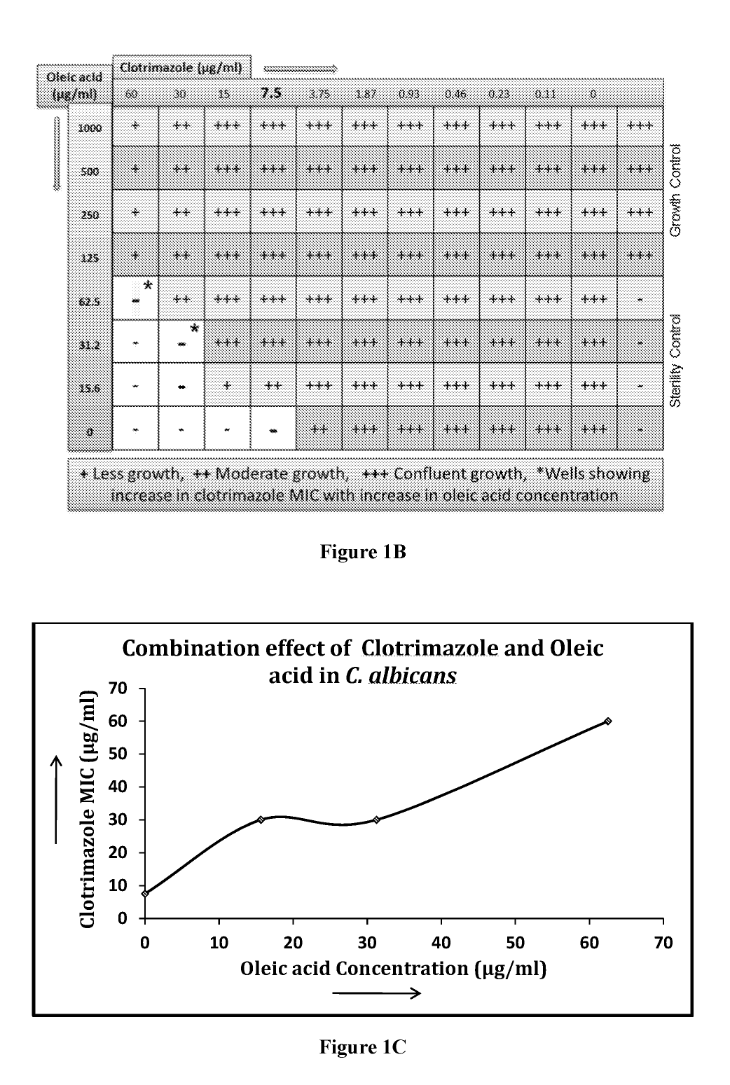

The synergy is measured by determining the fractional inhibitory concentration (FIC) value of the combination. This experimental set up, called checkerboard method, allows measurement of a desired effect (inhibition of fungal growth in this case) at different combinations of various concentrations of the two agents (antifungal agent and fatty acid/ester). A schematic representation of the layout is presented in FIG. 1A. As shown in FIG. 1A, test agent 1 is serially diluted through the wells horizontally (left to right) and test agent 2 is serially diluted through the wells vertically down. No agent is added to the penultimate column on the right and the bottom row. The final column on the right is used for growth control (only inoculum in broth) and sterility control (broth alone, no inoculum). Inoculum of relevant fungal strain were added to the wells with various drug combinations and observed for growth inhibition at the end of the incubation period set by protocol. For any combination wherein growth inhibition was observed at concentrations below the individual MICs of each agent, calculations were performed to determine the fractional inhibitory concentration. FIC value for each agent in a particular well of the checkerboard layout was calculated by dividing the agent concentration in that well by the established MIC value of the agent against the test organism (Hsieh et al., Synergy assessed by checkerboard: A critical Analysis, Diagn. Microbiol. Infect Dis. (1993) 16:343-349). FIC values for both agents in a particular well were calculated in this way followed by determination of the FIC index (sum of the FICs of each drug in the concerned well). Combinations that gave FIC indices less than 1 are designated as “synergistic” based on the guidelines from the literature [Zhang et al., Synthesis of novel sulfonamide azoles via C-N cleavage of sulfonamides by azole ring and relational antimicrobial study, New J Chem. (2015) 39:5776-5796 and Meletiadis et al., Defining Fractional Inhibitory Concentration Index Cutoffs for Additive Interactions based on self-drug combinations, Antimicrob. Agents Chemother. (2010) 54(2): 602-609].

Antifungal agent as used herein includes, but is not limited to N-hydroxy pyridone class like piroctone olamine, ciclopirox olamine; imidazoles like ketoconazole, climbazole, miconazole nitrate, fluconazole, econazole, saperconazole, oxiconazole, clotrimazole, bifonazole, butoconazole, fenticonazole, isoconazole, omoconazole, sertaconazole, sulconazole, tioconazole, luliconazole, chlormidazole, croconazole, eberconazole, omoconazole, isoconazole, neticonazole; triazoles like albaconazole, efinaconazole, fosfluconazole, epoxiconazole, fluconazole, isavuconazole, itraconazole, posaconazole, propiconazole, ravuconazole, terconazole, voriconazole, hexaconazole; allylamines like terbinafine, amorolfine, naftifine; benzylamines like butenafine, thiazoles like abafungin; echinocandins like caspofungin, anidulafungin, micafungin; polyenes like amphotericin B, natamycin and nystatin; N-hydroxy pyrithione like zinc pyrithione; other antifungals like tavaborole, flucytosine, griseofulvin, selenium disulfide, salicylic acid, sulfur, tar preparations and hinokitol. Other antifungal agents described in the present disclosure and known in the art are also used/applicable in the compositions of the present invention.

The medium carbon chain fatty acids, as used herein includes saturated or mono, di or poly unsaturated C-1 to C-14 (also interchangeably referred as C1 to C14) fatty acids, including propionic acid (propanoic acid), butyric acid (butanoic acid), valeric acid (pentanoic acid), caproic acid (hexanoic acid), enanthic acid (heptanoic acid), caprylic acid (octanoic acid), pelargonic acid (nonanoic acid), capric acid (decanoic acid), undecylic acid (undecanoic acid), lauric acid (dodecanoic acid), tridecylic acid (tridecanoic acid) and myristic acid (tetradecanoic acid), and esters/derivatives of said saturated or mono, di or poly unsaturated C-1 to C-14 fatty acids thereof including but not limited to mono, di and tri-esters of propylene glycol and glycerol and their derivatives, or any combinations thereof. In some embodiments of the present disclosure, the saturated or unsaturated medium chain fatty acid having carbon chain length ranging from C-1 to C-14 is caprylic acid, undecylenic acid, lauric acid, their esters, or any combinations thereof. In other embodiments, the saturated or unsaturated medium chain fatty acid esters/derivatives having carbon chain length ranging from C-1 to C-14 is a mono, di or tri ester of glycerol, propylene glycol and derivatives, or any combinations thereof.

As demonstrated by the data of the present disclosure, the exemplary saturated or unsaturated medium chain fatty acids and their esters show synergistic behavior with the antifungal agents. Accordingly, in some embodiments, the antifungal agent is selected from a group comprising allylamine class of antifungal agents, benzylamine class of antifungal agents, azole class of antifungal agents, echinocandin class of antifungal agents, polyene class of antifungal agents, N-hydroxy pyridone class of antifungal agents, N-hydroxy pyrithione like zinc pyrithione, and any combinations thereof, with the said medium chain fatty acids/esters thereof.

As used herein, the term “excipient” or “excipients” in the present compositions/formulations refer to other ingredient(s)/component(s) excluding the antifungal agent and fatty acids or their esters described herein. Such excipient includes classes selected from but not limited to additives, solvents, oils, emulsifiers, surfactants, polymers, stabilizers, other active agent(s) and any combinations thereof. Exemplary examples of the excipients are described throughout the disclosure. Further, any excipient generally known in the art for pharmaceutical or cosmetic applications is within the purview of the present disclosure/compositions/formulations.

The excipients in some embodiments are selected from a group comprising paraffin, thickeners selected from bentonite and cellulose, antioxidants selected from butylated hydroxyanisole (BHA), tert-butylhydroquinone (TBHQ), ferulic acid, tocopherol acetate or any combination of antioxidants thereof, perfumes or fragrances, essential oils, pH adjusters selected from triethanolamine, sodium hydroxide, inorganic or organic acids including citric acid, lactic acid, succinic acid, acetic acid, fumaric acid, glycolic acid, benzoic acid, bases, salts buffers or any combination of pH adjusters thereof, herbal extracts selected from amla fruit extract, arnica extract and brahmi extract, preserving agents selected from butylated hydroxytoluene (BHT), methyl p-hydroxybenzoate, propyl p-hydroxybenzoate, sorbic acid or any combination of preserving agents thereof, hair conditioning substances, hair care adjuncts selected from taurine, caffeine, minoxidil, azelaic acid, marine cartilage, hydrolysed keratin, biotin, niacin, panthenol, vitamin B6, zinc, copper, peptides, horsetail silica, beta sitosterols, pycnogenol, PABA, green tea extract, folic acid, iron, L-cysteine, magnesium, ginseng or any combination of hair care adjuncts thereof, skin care adjuncts selected from proteins, vitamins including A, B, C, D, E and K, trace metals including zinc, calcium and selenium, moisturizers. LTV absorbers including paraminobenzoic acid (PABA), titanium dioxide, zinc oxide, anti-irritants including steroids and non-steroidal anti-inflammators, botanical extracts including aloe vera, chamomile, cucumber extract, ginkgo biloba, ginseng and rosemary, absorbents including aluminum starch octenylsuccinate, kaolin, corn starch, oat starch, cyclodextrin, talc and zeolite, skin bleaching and lightening agents including hydroquinone and niacinamide lactate, humectants including sorbitol, urea and manitol, exfoliants, cooling agents like menthol, menthol derivatives, WS 3, WS-5, WS 14, WS 23, MHB, frescolat MGA, 2S MPD, coolcat P, WS 30, PM 38, skin conditioning agents including aloe extract, allantoin, bisabolol, shea butter, ceramides, sphingosine, dimethicone, hyaluronic acid and dipotassium glycyrrhizate, natural components including oatmeal, or any combination of skin care adjuncts thereof; emollient, dyestuffs, moisturizers, vitamins, sphingoceryls, sunscreens, co-surfactants, foaming agents, co-emulsifiers, viscosity modifiers, suspending agents, potentiating agents, pearlizing agents, cooling agents, ionic strength modifiers and oil-soluble polymers which are compatible with the base oil or skin care agents or both including skin-nutrient agents, anti-wrinkle agents, light and dust protectors, and combinations thereof; solvent selected from a group comprising ethanol, isopropyl alcohol, butanol, C-1 to C-6 lower aliphatic alcohols, lower alkyl acetate, ethers, carboxylic acid, derivatives containing carbon chain length less than C15, fatty alcohols selected from a group comprising undecanol, oleyl alcohol and lauryl alcohol, or any combination of solvents thereof; emulsifier selected from a group comprising steareth-2, steareth-21, poloxamer, macrogolcetostearyl ether 20, cetyl alcohol cetearths, ceteth, isoceteths, laureths, oleths, steareths, lauramide DEA, linoleamide DEA or any combination of emulsifiers thereof; surfactant is selected from a group comprising poloxamer, PEG-2 stearyl ether, PEG-21 stearyl ether, pluoronic F127 (poloxamer), polyoxyl 20 cetosteryl ether, sodium laryl ether sulphate, coco monoethanolamide, cocamidopropylbetain, sodium docusate, ammonium lauryl sulphate, coco glucoside, lauryl glucoside, decyl glucoside, caprylyl capryl glucoside, sodium cocoyl glutamate, disodium cocoyl glutamate, sodium lauroamphoacetate, sodium cocoamphoacetate, disodium cocoamphoacetate, disodium laureth sulfosuccinate, sodium methyl cocoyl taurate, sodium methyl oleoyl taurate, sodium cocoyl isethionate, ammonium cocoyl isethionate, sodium lauryl glucose carboxylate, sodium lauroyl lactylate, sodium lauroyl sarcosinate, sodium lauroyl methyl isethionate, sodium cocoyl glycinate, or any combination of surfactants thereof; surfactant and co-surfactant blend selected from Iselux SLC comprising sodium lauroyl methyl isethionate, sodium lauroamphoacetate, cocamide MIPA and water, Miracare SLB 365/N comprising sodium trideceth sulfate, sodium lauroamphoacetate, cocamide MEA, sodium chloride, methylisothiazolinone and water, or any combination of blends thereof; oil includes natural or synthetic oils is selected from a group comprising eucalyptus oil, rosemary oil, pine needle oil, tea tree oil, sage oil, cinnamon oil, lemon oil, citronella oil, lime oil, orange oil, peppermint oil, spearmint oil, wintergreen oil, sweet birch oil, clove leaf oil, camphor oil, cardamon oil, cedar leaf oil, sweet birch oil, paraffin oil, silicone oil or any combination of oils thereof; polymer is selected from a group comprising PEG, cellulose derivatives, acrylic based polymers, poloxamers, and combinations thereof; stabilizer is selected from a group comprising metal chelators, acrylic and cellulose derivatives, sodium carboxy methyl cellulose, poly vinyl alcohol, xanthan gum, guar gum, locust bean gum and combinations thereof; and active agent is selected from a group comprising pharmaceutical active, OTC active, anti-bacterial agent including benzothenium chloride, anti-inflammatory agent, skin penetration enhancer and combinations thereof.

As used herein, the antifungal compositions can be obtained/formulated in any form. For instance, the present the antifungal compositions are in typical particle form, solubilized form, dispersed form, nanoparticle form or any combination thereof. It is to be understood that the present antifungal compositions are not limited by any particular form and all forms of the composition is within the scope of the present invention.

In some embodiments, the antifungal agent in the present compositions is selected from a group comprising zinc pyrithione, piroctone olamine, terbinafine, butenafine, clotrimazole, ketoconazole, efinaconazole, luliconazole, bifonazole, caspofungin, amphotericin B and any combinations thereof.

The present disclosure specifically addresses the need of the art by providing antifungal/antimicrobial compositions which does not enhance fungal growth/infection, and synergistically improves fungal growth inhibition/treatment of fungal infection. Said antifungal/antimicrobial compositions are specifically devoid of long chain C-15 or greater fatty acids or their esters which are shown as promoters of fungal growth.

The present invention is directed to a variety of antifungal formulations comprising at least one antifungal agent described herein and at least one medium carbon chain fatty acid (C-1 to C-14) or ester thereof, with/without excipient(s).

The present invention is further directed to a variety of antifungal formulations comprising at least one antifungal agent described herein and at least one medium carbon chain fatty acid C-1 to C-14 or ester thereof, with/without excipient(s).

The present invention is also directed to a variety of antifungal formulations comprising at least one antifungal agent described herein and at least one medium carbon chain fatty acid of C-1 to C-10 or ester thereof, with/without excipient(s).

The present invention further provides antifungal formulations comprising at least one antifungal agent described herein described herein and at least one C-8 fatty acid or ester thereof, with/without excipient(s). In exemplary embodiments, antifungal formulations comprising one or more antifungal agents described herein and propylene glycol monocaprylate, with/without excipient(s) is provided. In other embodiments, antifungal formulations are provided comprising one or more antifungal agents described herein along with caprylic acid, with/without excipient(s). In yet another embodiment, antifungal formulations are provided comprising one or more antifungal agents described herein and propylene glycol monolaurate, with/without excipient(s).

In one embodiment, the antifungal agent used in the composition of the present invention is piroctone olamine. In another embodiment, the antifungal agent is ketoconazole. In yet another embodiment of the present invention, the composition comprises a combination of piroctone olamine and ketoconazole.

In one embodiment, the antifungal agent used in the composition of the present invention is zinc pyrithione. In yet another embodiment of the present invention, the composition comprises a combination of zinc pyrithione and ketoconazole.

In another embodiment, the anti-dandruff/anti-fungal agents of the present compositions include ketoconazole, climbazole, selenium sulfide, piroctone olamine, ciclopirox olamine, zinc pyrithione, or any combinations thereof wherein said antifungal agent may be present in the solubilized form or dispersed form or in the particle or nanoparticle form. Other anti-fungal agents from similar class are known to the art may also be used in the formulation.

In one embodiment, the antifungal agent used in the composition of the present invention is ketoconazole.

In one embodiment, the antifungal agent used in the composition of the present invention is clotrimazole.

In one embodiment, the antifungal agent used in the composition of the present invention is luliconazole.

In one embodiment, the antifungal agent used in the composition of the present invention is efinaconazole.

In one embodiment, the antifungal agent used in the composition of the present invention is bifonazole.

In one embodiment, the antifungal agent used in the composition of the present invention is terbinafine.

The present disclosure also provides shampoo formulations containing antifungal agent and medium carbon chain fatty acids or esters described herein along with excipient selected from at least one, two or more anti-dandruff agents, at least one or two oil components, viscosity modifiers, conditioning agent, stabilizers, emulsifiers and surfactants selected from the group of mild sulfate or sulfate free or combinations of anionic and zwitterionic or anionic and neutral or anionic, neutral and zwitterionic surfactants that can form structured multilamellar liquid surfactant system that can deform to form multi lamellar vesicles/spherulites when sheared or diluted.

In another aspect of the invention, the shampoo compositions of the present disclosure is used for the treatment of seborrheic dermatitis.

The shampoo compositions of the present invention may further contain excipients including additives known in the art. For example, the shampoo compositions may comprise excipients selected from preservatives, perfumes, pH adjusting agents, colorants one or more viscosity modifiers, humectants, conditioners, bactericides, surfactants etc. In addition to said excipients, the shampoo composition may further contain alcohols, ketones and other solvents or herbal extracts, fruit extracts, vitamins, pigments. In an embodiment, the surfactant includes sulfate free surfactants selected from a group comprising coco glucoside, lauryl glucoside, decyl glucoside, caprylyl capryl glucoside, sodium cocoyl glutamate, disodium cocoyl glutamate, sodium lauroamphoacetate, sodium cocoamphoacetate, disodium cocoamphoacetate, disodium laureth sulfosuccinate, sodium methyl cocoyl taurate, sodium methyl oleoyl taurate, sodium cocoyl isethionate, ammonium cocoyl isethionate, sodium lauryl glucose carboxylate, sodium lauroyl lactylate, sodium lauroyl sarcosinate, sodium lauroyl methyl isethionate, sodium cocoyl glycinate and others surfactants as known in the art. In another embodiment, the surfactant includes mild surfactants that can form structured multilamellar liquid surfactant system which deform to form multi lamellar vesicles/spherulites when sheared can be used. In yet another embodiment, surfactant includes an individual surfactant or a blend of various surfactants in right proportion. Exemplary examples of surfactant and co-surfactant blends include Iselux SLC (sodium lauroyl methyl isethionate, sodium lauroamphoacetate, cocamide MIPA and water) and Miracare SLB 365/N (sodium trideceth sulfate, sodium lauroamphoacetate, cocamide MEA, sodium Chloride, methylisothiazolinone and water) where combination of different surfactants at particular ratios in the presence of particular concentration of electrolyte is responsible for formation of structured multilamellar liquid surfactant system that helps in dispersing and stabilizing high levels of oil, fragrance and different high density particles such as mica, pigments, zinc pyrithione, selenium sulfide etc. into the formulation. Structured surfactant system provides excellent stability of the formulation over wide temperature range while retaining good foaming performance in the presence of oil and improved persistence of fragrance on hair and skin while maintaining high conditioning performance.

The present invention particularly describes ketoconazole, clotrimazole, luliconazole, efinaconazole, bifonazole and terbinafine based topical cream or gel or emulgel or lotion formulations containing at least one fatty acid having carbon chain length of C-1 to C-14 or ester thereof, along with excipients selected from at least one or two oil components, surfactants, co-surfactants, viscosity modifiers or gelling agents, emollients, skin penetrating agents, conditioning agents, stabilizers, emollients to finally obtain spreadable stable topical formulations. The pH of the formulation is adjusted by suitable pH modifier to maintain final pH of 5-7 and preferably pH 6-7. In the formulation, API (antifungal agent) and the medium chain fatty acid or ester thereof is either completely solubilized form or present in oil globules with particular size distribution ranges from 100-1000 nm, and preferably 100-500 nm that would help better absorption of the active through skin to achieve improved pharmacokinetics and pharmacodynamics.

In the present invention, the topical formulations including cream, gel or lotion is used for the diagnosis and management of various skin fungal infections/fungal growth caused by but not limited to pathogens including Malassezia, Candida and dermatophytes such as Trichophyton wherein the fatty acid or esters thereof in said composition is restricted to saturated or unsaturated medium chain fatty acids (C1 to C14), esters and derivatives thereof. In exemplary embodiments, the fatty acid is a molecule having a carbon chain length of C11 to C14, or corresponding esters and/or derivatives thereof. In other embodiments, the fatty acid is a molecule having a carbon chain length of C1 to C10, or corresponding esters and/or derivatives thereof. In another exemplary embodiment, the fatty acid is a molecule having a carbon chain length of C8, or corresponding esters and/or derivatives thereof. In preferred embodiments, the compositions of the present disclosure include one or more fatty acid selected from caprylic acid, capric acid, undecylenic acid, lauric acid and the respective mono/di and tri ester derivatives of propylene glycol and glycerol. The percentage of fatty acids and/or corresponding esters or derivatives in the composition ranges from 1-10%, and depending on the oil percentage the concentration of the excipients including surfactants, co-surfactants and emulsifiers vary to finally obtain hydrophobic lipophilic balanced (HLB) stable formulations. Different nature of gelling agent(s) (excipient) at different concentrations is added into the composition to obtain particular viscosity of the formulation as desired. Gelling agent can be selected from carbopol or hydroxyethyl cellulose (HEC) or any other known agent, or any combinations thereof within the concentration ranges from 0.05-1% and more preferably 0.05-0.5%.

In an embodiment of the present disclosure, luliconazole is present along with medium chain fatty acids and/or corresponding esters or derivatives in completely solubilized form in the form of lotion wherein the said active is stabilized by particular concentration of surfactants, co-surfactants, emulsifiers, stabilizing agent and emollients to form transparent to opaque lotion and preferably transparent formulation. The composition is either devoid of alcohol or with minimum percentage of alcohol ranging from 1-20%, and more preferably 1-10% along with other solubilizers like 1,3-propanediol or diethylene glycol mono ethylether or diisopropyl adipate or any other solubilizer at particular ratios to finally obtain either water based or oil based transparent to opaque, and preferably transparent stable topical lotion.

In another embodiment of the present disclosure, luliconazole and efinaconazole along with medium chain fatty acids and/or corresponding esters or derivatives based nail lacquer or nail solution are made for the treatment or management of onychomycoses. The nail lacquer of the present invention forms a transparent solution including an organic film former which in general any kind of biocompatible organic solvents that upon application to the nails, evaporate, leaving a relatively water permeable film. The amount of solvent in the nail lacquer or solution composition of the present invention is sufficient to solubilize and dissolve the film-forming compounds as well as the active (antifungal agent) at a particular concentration. The solvents include alcohol, butyl acetate, ethyl acetate or any other solvents known in the prior art. Film-forming compounds include polymers and copolymers of vinyl acetate, polymers and copolymers of acrylic or methacrylic acid (e.g., polymethyl methacrylate) polyvinylacytel and polyvinylbutyrals. The plasticizers like triacetin or any other known in the art can be employed. The above composition is used for the treatment of Candida and various tinea infections wherein said composition is restricted to saturated or unsaturated medium chain fatty acids, esters and derivatives thereof as described above. In exemplary embodiments, such medium chain fatty acids are selected from caprylic acid, capric acid, undecylenic acid, lauric acid and the respective mono/di and tri ester derivatives of propylene glycol and glycerol.

The amount of antifungal agent(s) used in the compositions of the present invention is in the range of from about 0.01% to 20% by weight of the total composition. In one embodiment, the antifungal agent is in the range from about 0.01% to about 10% by weight of the total composition. In a further embodiment, the antifungal agent is in the range of from about 0.01% to about 5% by weight of the total composition. In yet another embodiment, the antifungal agent is in the range of about 0.01% to about 2% by weight of the total composition.

As used herein, excipient includes, but is not limited to, solvents, surfactants and additives used in pharmaceutical and cosmetic formulations. The amounts of excipients used in the compositions of the present invention is in the range of about 45% to about 99% by weight of the total composition.

In an embodiment of the present disclosure, the excipient is an oil and includes, but is not limited to, paraffin oil, silicone oils, terpenes, fatty alcohols, dibutyl adipate, dioctyl adipate, or any combination of oils thereof. Essential oils as used herein include, but are not limited to, natural and synthetic oils such as eucalyptus oil, rosemary oil, pine needle oil, tea tree oil, sage oil, cinnamon oil, lemon oil, citronella oil, lime oil, orange oil, peppermint oil, spearmint oil, wintergreen oil, sweet birch oil, clove leaf oil, camphor oil, cardamon oil, cedar leaf oil, sweet birch oil and other oils known to a skilled person in the art. The amount of oil used in the compositions of the present invention is in the range of about 0.5% to about 99% by weight of the total composition, more preferably 50% to 99% when formulated as oil, 5% to 50% when formulated as cream/ointment or 0.5% to 20% when formulated as gel/serum/spray.

In another embodiment, excipient is solvent and includes, but is not limited to, lower aliphatic alcohols, such as, for example, ethanol, isopropyl alcohol, butanol and the likes, lower alkyl acetate, ethers, fatty alcohols such as undecanol, oleyl alcohol, lauryl alcohol or combinations thereof.

In another embodiment, excipient is additive and includes, but is not limited to, thickeners, antioxidants, perfumes/fragrances, essential oils, pH adjusters, herbal extracts, preserving agents, hair conditioning substances, hair care adjuncts, skin care adjuncts, emollient, dyestuffs, moisturizers, vitamins, sphingoceryls, sunscreens, surfactants, oil-soluble polymers which are compatible with the base oil and/or skin care agents including skin-nutrient agents, anti-wrinkle agents, light and dust protectors, or any combination of additives thereof. For example, compositions of the present invention may contain additives such as thickeners (for example, bentonite, cellulose and the like), antioxidants (for example, butylated hydroxytoluene (BHT), butylated hydroxyanisole (BHA), tert-butylhydroquinone (TBHQ), ferulic acid, vitamin A, vitamin E (tocopherol)), preservatives (for example, methyl p-hydroxybenzoate or propyl p-hydroxybenzoate, sorbic acid and the like), hair care ingredients (for example, fatty alcohols, peptides, proteins, vitamins and mixtures thereof), light protective agents or sunscreens (for example, p-methoxycinnamic acid isoamyl ester and the likes).

In yet another embodiment, the excipient is surfactant and includes, but are not limited to, cetearths, ceteth, isoceteths, laureths, oleths, steareths, lauramide DEA, linoleamide DEA and other surfactants which are suitable for topical application.

In still another embodiment, the pH adjusters include, but are not limited to, inorganic or organic acids (e.g., citric acid, lactic acid, succinic acid, acetic acid, fumaric acid, glycolic acid, benzoic acid), bases, salts and/or buffers thereof. In an embodiment, the herbal extracts as used herein include, but are not limited to, amla fruit extract, arnica extract, brahmi extract and others known to the art-skilled. In another embodiment, the hair care adjuncts as used herein include, but are not limited to, ingredients beneficial in the treatment of hair loss or the promotion of hair growth such as taurine, caffeine, minoxidil, azelaic acid, marine cartilage, hydrolysed keratin, biotin, niacin, panthenol, vitamin B6, zinc, copper, peptides, horsetail silica, beta sitosterols, pycnogenol, PABA, green tea extract, folic acid, iron, L-cysteine, magnesium, ginseng and others known to the art-skilled. In yet another embodiment, the skin care adjuncts as used herein include, but are not limited to, those that are beneficial for the treatment of various skin conditions (like dry skin, oily skin, fine lines, pigmentation, etc.) such as proteins, vitamins (e.g., A, B, C, D, E, and K), trace metals (e.g., zinc, calcium and selenium), moisturizers (e.g., emollients, humectants, film formers, occlusive agents, and agents that affect the natural moisturization mechanisms of the skin), LTV absorbers (physical and chemical absorbers such as paraminobenzoic acid (PABA), titanium dioxide, zinc oxide, etc.), anti-irritants (e.g., steroids and non-steroidal anti-inflammatories), botanical extracts (e.g., aloe vera, chamomile, cucumber extract, ginkgo biloba, ginseng, and rosemary), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., sorbitol, urea, and manitol), exfoliants, skin conditioning agents (e.g., aloe extracts, allantoin, bisabolol, ceramides, dimethicone, hyaluronic acid, and dipotassium glycyrrhizate) and other natural components (e.g., oatmeal) known to the art-skilled.

Another embodiment of the present invention provides use of the antifungal compositions described herein as a medicament, more particularly for managing fungal growth or treating fungal infections and associated complications/conditions therein.

As used herein, the terms “manage”, “managing”, “management”, “treat”, “treating” or “treatment” of fungus growth or fungus infection refers to both medical or non-medical indications. In one aspect, these terms cover one or more aspects including but not limiting to preventing or reducing growth of fungi, inhibiting further growth of fungi, eliminating the grown fungi at the infected area/site, providing symptomatic relief to a subject in need thereof, successfully eliminating the infection, curing the fungal infection, preventing recurrence of fungal infection, curing drug resistant fungal infections, and treatment of fungal infections in clinical non-responders and patients with barrier defects. It is to be understood that the antifungal compositions of the present invention achieves one or more of the above described effects, and includes any additional effects/activity known to a person skilled in the art. In an exemplary embodiment of the present disclosure, the above terms cover any antifungal treatment in a mammal, including human.

Yet another embodiment of the present invention provides methods for the treatment of fungal infections comprising administering to a subject/patient in need thereof an antifungal composition of the present invention.

In some embodiments, the fungal infection is a resistant fungal infection. In exemplary embodiments, the fungal infection is resistant to the antifungal agent or class of antifungal agents included in the antifungal composition comprising the said antifungal agent and a medium chain fatty acid or its ester that show synergistic antifungal activity for treatment of the said resistant fungal infection.

The antifungal compositions of the present invention are used in the treatment of diseases associated with, including but not limited to, Malassezia spp. (e.g., M. furfur, M. pachydermatis, M. globosa, M. restricta, M. slooffiae, M. sympodialis, M. nana, M. yamatoensis, M. dermatis, M. obtusa, M. japonica, M. caprae, M. cuniculi, M. equine, and M. arunalokei) Trichophyton spp. (e.g., T. rubrum, T. mentagrophyte, T. interdigitale, T. tonsurans, T. schoenleinii, T. violaceum, T. abissinicum, T. balcaneum, T. circonvolutum, T. concentricum, T. eboreum, T. errinacei, T. fischeri, T. fluviomuniense, T. glabrum, T. gourvilii, T. kanei, T. kuryangei, T. megninii, T. pedis, T. proliferans, T. raubitschekii, T. redellii, T. rodhainii, T. simii, T. soudanense, T. thuringiense, T. verrucosum, T. violaceum, Trichophyton yaoundei), Candida spp. (C. albicans, C. glabrata, C. guilliermondii, C. krusei, C. lusitaniae, C. parapsilosis, C. tropicalis, C. colliculosa, C. dubliniensis, C. famata, C. haemulonii, C. inconspicua, C. intermedia, C. kegir, C. lipolytica, C. metapsilosis, C. norvegensis, C. orthopsilosis, C. pelliculosa, C. pulcheirima, C. rugose, C. utilis, C. viswanathii, C. zeylanoides), Microsporum spp. (M. audouinii, M. canis, M. amazonicum, M. boullardii, M. cookie, M, distortum, M. diboisii, M. equinum, M. feimgineum, M. fulvum, M. gallinae, M. gypseum, M. langeronii, M nanum. M persicolor, M. praecox, M. ripariae, M. rivalieri), Epidermaphyton spp (E. floccosum) and other non-dermatophytes including but not limited to Aspergillus spp. (A. fumigates, A. flavus, A. nidulans, A. terreus, A. lentulus, A. niger, A. alliaceus, A. arvii, A. brevipes, A. calidoustus, A. conjunctus. A, deflectus, A. duricaulis, A. emericella, A. fischerian. A. fumigatiqffinis, A. fumisvnnematus, A. granulosus, A. novofumigatus, A. panamensis, A. quadrilineatus, A. udagawae, A. unilateralis, A. ustus) and Cryptococcus spp. (C. neoformans, C. gatti, C. albidus, C. bacillisporus, C. decagatti, C. deuterogatti, C. laurentii, C. tetragatti, C. uniguttulatus). Compositions of the present invention are intended for use in the treatment of diseases including but not limited to tinea pedis, tinea capitis, tinea cruris, tinea glabrosa, tinea corporis, tinea unguium, tinea faciei, tinea manuum, piedra, pityriasis capitis, pityriasis vesicolor, pityrosporum folliculitis, seborrheic dermatitis, diaper rash, scalp seborrheic dermatitis, cutaneous candidiasis, onychomycosis, candida folliculitis, skin fungal infections associated with barrier defects as in atopic dermatitis, xerotic eczema and psoriasis, otomycosis, mucosal candidiasis and deep tissue infections including but not restricted to biofilm forming/non-forming fungal infections associated with surgically implanted devices like indwelling intravenous catheters, orthopaedic devices, urinary catheters, intrauterine devices, dialysis vascular grafts and central nervous system implants.

The compositions of the present invention are also of veterinary use in the topical treatment of dermatological fungal infections.

Compositions of the present invention provide better retention and penetration of antifungal agent onto the hair, skin, scalp and nails. Accordingly, the present invention provides compositions and methods of managing fungus growth or treating fungal infections of the skin, scalp, hair or nail. In one embodiment of the present invention, the antifungal composition is topical hair oil. In another embodiment, the antifungal composition of the present invention is anti-dandruff oil. In yet another embodiment, the composition of the present invention is a hair gel. In another embodiment, the antifungal composition of the present invention is anti-dandruff shampoo. In another embodiment, the antifungal composition of the present invention is anti-dandruff hair serum. In a further embodiment, the composition of the present invention is a nail varnish.

The compositions of the present invention is employed for the purpose of topical and/or local administration in the form of oils, creams, lotions, serums, gels, ointments, foams, sprays, aerosols, coating on implants, silicon tubes, catheters, sutures and the likes.

Exemplary In Vitro Synergistic Combinations

In some embodiments, the synergistic combination of the present invention comprises caprylic acid or its ester derivatives with an antifungal agent selected from a group comprising allylamine class of antifungal agents, benzylamine class of antifungal agents, azole class of antifungal agents, echinocandin class of antifungal agents, polyene class of antifungal agents, N-hydroxy pyridone class, N-hydroxy pyrithione like zinc pyrithione, and selenium sulfide, or any combinations thereof.

In some embodiments, the synergistic combination of the present invention comprises undecylenic acid with an antifungal agent selected from a group comprising allylamine class of antifungal agents, benzylamine class of antifungal agents, azole class of antifungal agents, echinocandin class of antifungal agents, polyene class of antifungal agents, N-hydroxy pyridone class, N-hydroxy pyrithione like zinc pyrithione, and selenium sulfide, or any combinations thereof.

In some embodiments, the synergistic combination of the present invention comprises lauric acid or its ester derivative with an antifungal agent selected from a group comprising allylamine class of antifungal agents, benzylamine class of antifungal agents, azole class of antifungal agents, echinocandin class of antifungal agents, polyene class of antifungal agents, N-hydroxy pyridone class, N-hydroxy pyrithione like zinc pyrithione, and selenium sulfide, or any combinations thereof.

Exemplary Antifungal Compositions Comprising Medium Chain Fatty Acids/Esters Thereof with Antifungal Agents which Impart Synergistic Antifungal Activity

The compositions of the present disclosure are devoid of C-15 or greater fatty acids and/or esters thereof. The compositions of the present disclosure comprise C-1 to C-14 fatty acids and/or esters thereof. In exemplary embodiments, compositions of the present disclosure comprise C-11 to C-14 fatty acids and/or esters thereof, and are devoid of C-15 or greater fatty acids and/or esters thereof. In other exemplary embodiments, the compositions of the present disclosure comprise C-1 to C-10 fatty acids and/or esters thereof, and are devoid of C-15 or greater fatty acids and/or esters thereof. In additional embodiments, the compositions of the present disclosure comprise C-8 fatty acid and/or esters thereof, and are devoid of C-15 or greater fatty acids and/or esters thereof.

In further exemplary embodiments, the compositions of the present disclosure comprise C-12 fatty acid and/or esters thereof, and are devoid of C-15 or greater fatty acids and/or esters thereof.

In some embodiments, the antifungal composition comprises caprylic acid or caprylic acid esters and an antifungal agent selected from various classes comprising allylamines, benzylamines, azoles, echinocandins, polyenes, N-hydroxy pyridones, N-hydroxy pyrithione like zinc pyrithione and selenium sulfide, and or any combinations thereof, wherein the composition is devoid of C-15 or greater fatty acids and/or esters thereof.

In further embodiments, the antifungal composition comprises propylene glycol monocaprylate (caprylic acid ester) and an antifungal agent selected from various classes comprising allylamines, benzylamines, azoles, echinocandins, polyenes, N-hydroxy pyridones, N-hydroxy pyrithione like zinc pyrithione and selenium sulfide, or any combinations thereof, wherein the composition is devoid of C-15 or greater fatty acids and/or esters thereof.

In other embodiments, the antifungal composition comprises glyceryl monocaprylate (caprylic acid ester) and an antifungal agent selected from various classes comprising allylamines, benzylamines, azoles, echinocandins, polyenes. N-hydroxy pyridones, N-hydroxy pyrithione like zinc pyrithione and selenium sulfide, or any combinations thereof, wherein the composition is devoid of C-15 or greater fatty acids and/or esters thereof.

In still other embodiments, the antifungal composition comprises undecylenic acid and an antifungal agent selected from various classes comprising allylamines, benzylamines, azoles, echinocandins, polyenes, N-hydroxy pyridones, N-hydroxy pyrithione like zinc pyrithione and selenium sulfide, or any combinations thereof, wherein the composition is devoid of C-15 or greater fatty acids and/or esters thereof.

In some embodiments, the antifungal composition comprises lauric acid or lauric acid ester and an antifungal agent selected from various classes comprising allylamines, benzylamines, azoles, echinocandins, polyenes, N-hydroxy pyridones, N-hydroxy pyrithione like zinc pyrithione, selenium sulfide and any combinations thereof, wherein the composition is devoid of C-15 or greater fatty acids and/or esters thereof.

In further embodiments, the antifungal composition comprises propylene glycol monolaurate (lauric acid ester) and an antifungal agent selected from various classes comprising allylamines, benzylamines, azoles, echinocandins, polyenes, N-hydroxy pyridones, N-hydroxy pyrithione like zinc pyrithione and selenium sulfide, or any combinations thereof, wherein the composition is devoid of C-15 or greater fatty acids and/or esters thereof.

In still other embodiments, the antifungal composition comprises glyceryl monolaurate (lauric acid ester) and an antifungal agent selected from various classes comprising allylamines, benzylamines, azoles, echinocandins, polyenes, N-hydroxy pyridones, N-hydroxy pyrithione like zinc pyrithione and selenium sulfide, or any combinations thereof, wherein the composition is devoid of C-15 or greater fatty acids and/or esters thereof.

Resistant Fungi

As noted herein, the compositions of the invention, in addition to being highly effective against drug susceptible fungi, are particularly useful for treatment of resistant fungal infections. Without wishing to be bound by a theory, the compositions of the invention are particularly useful for treatment of antifungal infections which are resistant to one or more conventional drugs used for treatment of fungal infections. For example, the compositions of the invention are particularly useful for treatment of fungal infections which are resistant to azoles, allylamines and benzylamines.

Thus, in one aspect, the invention provides an antifungal composition for treatment of resistant fungal infection(s) comprising at least one antifungal agent and at least one fatty acid having carbon chain length of C-1 to C-14 or ester thereof, wherein said composition shows in vitro synergistic antifungal activity.

In some embodiments, the fungus associated with the infection is resistant to the antifungal agent or class of antifungal agent comprised in the antifungal composition. Accordingly, the present compositions are devised such that antifungal agent along with at least one medium chain fatty acid (C-1 to C-14) or ester thereof provides successful activity/treatment of the said resistant fungal infection.

In some embodiments, the antifungal composition for treatment of resistant fungi infection(s) comprises at least one antifungal agent and at least one fatty acid or ester thereof having carbon chain length ranging from C-1 to C-14 (the combination of the antifungal agent and fatty acid or ester showing in vitro synergistic antifungal activity), and at least one excipient. In exemplary embodiments, the at least one fatty acid or ester thereof has a carbon chain length ranging from C-11 to C-14, or C-1 to C-10, or C-8, or C-11, or C-12, or C-13, or C-14, or any combination thereof. In another embodiment, the composition is devoid of C-15 or greater fatty acids and esters.

In some embodiments, the antifungal composition for treatment of resistant fungal infection(s) comprises at least one antifungal agent and at least one fatty acid or ester thereof having carbon chain length ranging from C-1 to C-10 (the combination of the antifungal agent and fatty acid or ester showing in vitro synergistic antifungal activity), and at least one excipient, wherein said composition is devoid of C-15 or greater fatty acids and esters.

In some embodiments, the fatty acid having carbon chain length ranging from C-1 to C-14 is caprylic acid. In some embodiments, the fatty acid ester having carbon chain length ranging from C-1 to C-14 is an ester of caprylic acid. In some embodiments, the caprylic acid ester is propylene glycol monocaprylate. In other embodiments, the caprylic acid ester is glyceryl monocaprylate. In some embodiments, the fatty acid having carbon chain length ranging from C-1 to C-14 is undecylenic acid. In some embodiments, the fatty acid having carbon chain length ranging from C-1 to C-14 is lauric acid. In some embodiments, the fatty acid ester having carbon chain length ranging from C-1 to C-14 is an ester of lauric acid. In some embodiments, the lauric acid ester is propylene glycol monolaurate. In other embodiments, the caprylic acid ester is glyceryl monolaurate.

As demonstrated by the data in the below examples, the exemplary medium chain fatty acid and their esters show synergistic behavior with all the tested antifungal agents against resistant fungi. Accordingly, in some embodiments, the antifungal agent in the present compositions can be selected from a group comprising allylamine class of antifungal agents, benzylamine class of antifungal agents, azole class of antifungal agents, N-hydroxy pyridone class of antifungals, echinocandin class of antifungals, polyene class of antifungals, N-hydroxy pyrithione like zinc pyrithione and combinations thereof.

In some embodiments, the antifungal agent is selected from a group comprising but not limited to N-hydroxy pyridone class like piroctone olamine, ciclopirox olamine; imidazoles like ketoconazole, climbazole, miconazole nitrate, fluconazole, econazole, saperconazole, oxiconazole, clotrimazole, bifonazole, butoconazole, fenticonazole, isoconazole, omoconazole, sertaconazole, sulconazole, tioconazole, luliconazole, chlormidazole, croconazole, eberconazole, omoconazole, isoconazole, neticonazole; triazoles like albaconazole, efinaconazole, fosfluconazole, epoxiconazole, fluconazole, isavuconazole, itraconazole, posaconazole, propiconazole, ravuconazole, terconazole, voriconazole, hexaconazole; allylamines like terbinafine, amorolfine, naftifine; benzylamines like butenafine, thiazoles like abafungin; echinocandins like caspofungin, anidulafungin, micafungin; polyenes like amphotericin B, natamycin and nystatin; N-hydroxy pyrithione like zinc pyrithione; other antifungals like tavaborole, flucytosine, griseofulvin, selenium disulfide, salicylic acid, sulfur, tar preparations and hinokitol. Other antifungal agents known to the art-skilled may also be used in the compositions of the present invention.

The present disclosure further provides antimicrobial/antifungal agents along with medium chain saturated and unsaturated fatty acids (C-1 to C-14) or ester derivatives thereof, or a synergistic combination of different antimicrobial/antifungal agents and medium chain saturated and unsaturated fatty acids (C-1 to C-14) or ester derivatives thereof to coat either in the solubilized form or nanoparticle form or in the hydrogel form on implants. The compositions are used for coating latex catheters and silicone catheters. In an exemplary embodiment, the coating process involves solubilization of antimicrobial/antifungal agents and medium chain saturated and unsaturated fatty acids (C-1 to C-14) or ester derivatives thereof in suitable solubilizer/emulsifier (excipient) at particular ratios to form permanent coating on latex Foley catheters and silicone catheters. In an embodiment, the emulsifiers and solubilizers used is selected from oleyl alcohol, N-methyl pyrrolidone, N-methyl pyrrolidine, PEG-12 dimethicone, glycerol, ethanol, diethtylene glycol monoethylether, alkylmethylsiloxane, cyclomethicone, dimethicone or dimethicnol 40 alone or any combinations thereof provided they are compatible to the silicone based medical fluid. The catheter is then immersed into the final solution containing drug and medical fluid (coating agent) and kept for about 3 minutes at room temperature. At the end, it is taken out of the solution and is hanged to drain solution from the catheters. In general, for curing, coated catheter is allowed to dry at about 25° C., at 55% relative humidity for about 24 hours. The coating is cured in about 24 hours followed by packaging and sterilization to obtain the final coated catheters. In case of hydrophilic coating process, different natural synthetic polymers alone or in combination with antifungal agents or antimicrobial agents and medium chain fatty acids (C-1 to C-14) or fatty acid ester derivatives thereof in the form of hydrogel are used to coat on catheters. Natural and synthetic polymers include polytetrafluoroethylene, polymerized styrene, hydroxyethylcellulose, carboxymethylcellulose, hydroxypropylcellulose, hypromellose, ethylcellulose alone or in combinations thereof for use as suitable hydrogel forming matrix.

Additional exemplary embodiments of the invention are further described by one or more of the following numbered paragraphs:
1. An antifungal composition comprising at least one antifungal agent, at least one medium chain fatty acid or ester thereof, and at least one excipient, wherein a synergistic fractional inhibitory concentration (FIC) index of the combination of the antifungal agent and the medium chain fatty acid or ester thereof is less than 1.
2. The antifungal composition of paragraph 1, wherein said at least one fatty acid or ester thereof is a fatty acid or ester thereof having a carbon chain length ranging from C-1 to C-14.
3. The antifungal composition of paragraphs 1 or 2, wherein the at least one fatty acid or ester thereof is a fatty acid or ester thereof having a carbon chain length ranging from preferably C-1 to C-10 or C-11 to C-14, and wherein said composition is devoid of C-15 or greater fatty acids and esters.
4. The antifungal composition of paragraphs 2 or 3, wherein the fatty acid or ester thereof having carbon chain length ranging from C-1 to C-14 is caprylic acid.
5. The antifungal composition of any one of paragraphs 2-4, wherein the fatty acid or ester thereof having carbon chain length ranging from C-1 to C-14 is an ester of caprylic acid.
6. The antifungal composition of any one of paragraphs 2-5, wherein the fatty acid or ester thereof having carbon chain length ranging from C-1 to C-14 is propylene glycol monocaprylate.
7. The antifungal composition of any one of paragraphs 2-5, wherein the fatty acid or ester thereof having carbon chain length ranging from C-1 to C-14 is glyceryl monocaprylate.
8. The antifungal composition of any one of paragraphs 1-7, wherein the fatty acid or ester thereof having carbon chain length ranging from C-1 to C-14 or C-11 to C-14 is lauric acid or it's ester selected from propylene glycol monolaurate, glyceryl monolaurate or a combination thereof; and wherein the antifungal agent is selected from the group comprising allylamine class of antifungal agents, benzylamine class of antifungal agents, azole class of antifungal agents, polyene class of antifungal agents, echinocandin class of antifungal agents, N-hydroxy pyrithione like zinc pyrithione, N-hydroxy pyridone like piroctone olamine, and any combinations thereof.
9. The antifungal composition of any one of paragraphs 1-8, wherein the antifungal agent is selected from the group consisting of terbinafine, butenafine, clotrimazole, ketoconazole, luliconazole, bifonazole, efinaconazole, amphotericin B, caspofungin, zinc pyrithione, piroctone olamine, and any combinations thereof.
10. The antifungal composition of any one of paragraphs 1-9, wherein the composition is formulated for the purpose of topical administration, local administration and/or systemic administration in the form of oils, creams, lotions, serums, gels, hydrogels, ointments, foams, sprays, aerosols, water based serum, coating on implants such as silicon tubes, catheters and sutures.
11. A method for managing fungal growth or treating a fungal infection, comprising administering an antifungal composition as defined in the above paragraphs to a subject in need thereof.
12. A method for treating a resistant fungal infection, comprising administering an antifungal composition as defined in the above paragraphs to a subject in need thereof, wherein fractional inhibitory concentration (FIC) index of a combination of the antifungal agent and the fatty acid or ester thereof in the composition is less than 1.
13. A hair oil composition comprising piroctone olamine, propylene glycol monocaprylate and one or more excipient selected from but not limiting to the classes of surfactants, co-surfactants, emulsifiers, stabilizing agents, gelling agents, conditioning agents, moisturizing agents, preservatives, fragrance, emollients, pH modifiers, colorants, antioxidants and combinations thereof, the said composition more specifically comprising of the excipients including ethanol, IPA, oleyl alcohol, paraffin, triacetin ethanol, cyclomethicone, tea tree oil, tocopherol acetate, BHT, phenoxyethanol, and pH modifiers and combinations thereof.
14. The hair oil composition defined in paragraph 13, comprising piroctone olamine at a concentration ranging from about 0.01% to 5%, propylene glycol monocaprylate at a concentration ranging from about 0.5% to 20% and the excipient at a concentration ranging from about 45% to 99%, of the total weight of the composition.
15. A shampoo composition comprising antifungal agent selected from piroctone olamine, zinc pyrithione or a combination thereof, and propylene glycol monocaprylate along with one or more excipient selected from but not limiting to the classes of surfactants, co-surfactants, emulsifiers, stabilizing agents, gelling agents, pearlizing agents, conditioning agents, moisturizing agents, preservatives, foaming agents, fragrance, emollients, pH modifiers, colorants, antioxidants and combinations thereof.
16. The shampoo composition defined in paragraph 14, comprising antifungal agent at a concentration ranging from about 0.01% to 20%, propylene glycol monocaprylate at a concentration ranging from about 0.5% to 10% and the excipient at a concentration ranging from about 45% to 99%, of the total weight of the composition.
17. A gel or cream composition comprising antifungal agent selected from ketoconazole, clotrimazole, luliconazole or terbinafine, and propylene glycol monocaprylate along with one or more excipient selected from but not limiting to the classes of solubilizers, surfactants, co-surfactants, emulsifiers, stabilizing agents, gelling agents, moisturizing agents, preservatives, fragrance, emollients, skin barrier repair agents, pH modifiers, penetration enhancers, cooling agents, antioxidants and combinations thereof.
18. The gel or cream composition defined in paragraph 17, comprising antifungal agent at a concentration ranging from about 0.01% to 10%, propylene glycol monocaprylate at a concentration ranging from about 0.5% to 10% and the excipient at a concentration ranging from about 45% to 99%, of the total weight of the composition.
19. A lotion composition comprising antifungal agent luliconazole or piroctone olamine, and propylene glycol monocaprylate along with one or more excipient selected from but not limiting to the classes of solubilizers, surfactants, co-surfactants, emulsifiers, stabilizing agents, gelling agents, moisturizing agents, preservatives, fragrance, emollients, pH modifiers, penetration enhancers, skin barrier repair agents, cooling agents, antioxidants, antibacterial agents (benzothenium chloride) and combinations thereof.
20. The lotion composition defined in paragraph 19, comprising antifungal agent at a concentration ranging from about 0.01% to 20%, propylene glycol monocaprylate at a concentration ranging from about 0.5% to 20% and the excipient at a concentration ranging from about 45% to 99%, of the total weight of the composition.
21. A solution based composition comprising efinaconazole, and propylene glycol monocaprylate along with one or more excipient selected from but not limiting to the classes of solvents, solubilizers, surfactants, co-surfactants, emulsifiers, stabilizing agents, gelling agents, moisturizing agents, preservatives, emollients, pH modifiers, penetration enhancers, antioxidants and combinations thereof.
22. The solution based composition defined in paragraph 21, comprising efinaconazole at a concentration ranging from about 0.5% to 20%, propylene glycol monocaprylate at a concentration ranging from about 0.5% to 20% and the excipient at a concentration ranging from about 45% to 99%, of the total weight of the composition.
23. A water based serum composition comprising piroctone olamine, and propylene glycol monocaprylate along with one or more excipient selected from but not limiting to the classes of solvents, solubilizers, surfactants, co-surfactants, emulsifiers, stabilizing agents, gelling agents, moisturizing agents, preservatives, emollients, pH modifiers, penetration enhancers, antioxidants, and combinations thereof, the said composition more specifically comprising of the excipients including PEG-12 dimethicone, oleth-20, laureth 23, water, Sensomer CI 50, PEG-120 methyl glucose trioleate, phenoxyethanol and combinations thereof.
24. The water based serum composition defined in paragraph 21, comprising piroctone olamine at a concentration ranging from about 0.01% to 20%, propylene glycol monocaprylate at a concentration ranging from about 0.5% to 20% and the excipient at a concentration ranging from about 45% to 99%, of the total weight of the composition.
25. The hair oil composition, shampoo composition, gel composition or water based serum composition defined in above paragraphs, wherein said compositions possess antifungal activity against Malassezia spp. selected from a group comprising M. furfur M. pachydermatis, M. globosa, M. restricta, M. slooffiae, M. sympodialis, M. nana, M. yamatoensis, M. dermatis, M. obtusa, M. japonica, M. caprae, M. cuniculi, M. equine, M. arunalokei and combinations thereof.
26. The hair oil composition, shampoo composition, gel composition or water based serum composition defined in above paragraphs, wherein said compositions manage or treat indications/fungal infections selected from a group comprising dandruff, sebhoreic dermatitis, or a combination thereof.
27. The cream composition, gel composition, lotion composition or solution based serum composition defined in above paragraphs, wherein said compositions possess antifungal activity against Trichophyton spp. selected from a group comprising T. rubrum, T. mentagrophyte, T. interdigitale, T. tonsurans, T. schoenleinii, T. violaceum, T. abissinicum, T. balcaneum, T. circonvolutum, T. concentricum, T. eboreum, T. errinacei, T. fischeri, T. fluviomuniense, T. glabrum, T. gourvilii, T. kanei, T. kuryangei, T. megninii, T. pedis, T. proliferans, T. raubitschekii, T. redellii, T. rodhainii, T. simii, T. soudanense, T. thuringiense, T. verrucosum, T. violaceum, Trichophyvton vaoundei and combinations thereof.
28. The cream composition, gel composition, lotion composition or solution based serum composition defined in above paragraphs, wherein said compositions manage or treat indications/fungal infections selected from a group comprising tinea, cutaneous candidiasis, onychomycosis or any combination thereof.

The following examples serve to further illustrate the present invention and are not to be construed to limit the scope of the present invention.

### EXAMPLES

### Example 1: Exemplary Synergistic Antifungal Combinations of Medium-Chain Fatty Acids/Esters (C-1 to C-14) with Various Antifungal Agents Against Drug-Susceptible and Drug-Resistant Filamentous Fungus and Yeast

Using the checkerboard layout experimental setup described above (FIG. 1A), various medium-chain fatty acids were tested for their ability to potentiate the activity of a known antifungal agent. Concentrations above and below the MIC of each test agent were tested using serial dilution. The same procedure was expanded to test combinations of various medium-chain fatty acids C-1 to C-14 (such caprylic acid, undecylenic acid and lauric acid) and esters thereof, with antifungal agents from various classes including azoles, allylamines, benzylamines, zinc pyrithione and piroctone olamine against both susceptible and resistant Trichophyton spp. (filamentous fungus) and Candida spp. (yeast).

Method:

Potentiation of the activity of antimycotic agents using ester derivatives of medium chain fatty acids was tested in a standard, well accepted in vitro assay system to check for synergy. This experimental set up, called checkerboard method, allows measurement of a desired effect (inhibition of fungal growth in this case) at different combinations of various concentrations of two agents. A chosen medium chain fatty acid (C-1 to C-14) or its ester derivative was tested for its ability to potentiate the activity of a known antifungal agent (various classes) using the checkerboard layout. Various concentrations above and below the MIC of each test agent were tested using serial dilution in Sabouraud dextrose broth. Inoculum of relevant fungal strain were added to the wells with various drug combinations and observed for growth inhibition at the end of the incubation period set by protocol. For any combination wherein growth inhibition was observed at concentrations below the individual MICs of each agent, calculations were performed to determine the fractional inhibitory concentration (FIC). FIC value for a drug in a particular well of the checkerboard layout is calculated by dividing the drug concentration in that well by the established MIC value of the drug against the test organism (Hsieh et al., Synergy assessed by checkerboard: A critical Analysis, Diagn. Microbiol. Infect Dis. (1993) 16:343-349). FIC values for both test agents in a particular well are calculated in this way followed by determination of the FIC index (sum of the FICs of each drug in the concerned well). Combinations that gave FIC indices less than 1 were designated “synergistic” based on the existing literature (Zhang et al Synthesis of novel sulfonamide azoles via C-N cleavage of sulfonamides by azole ring and relational antimicrobial study, New J Chem. (2015) 39:5776-5796 and Meletiadis et al., Defining Fractional Inhibitory Concentration Index Cutoffs for Additive Interactions based on self-drug combinations, Antimicrob. Agents Chemother. (2010) 54(2): 602-609).

Results:

The investigational medium chain fatty acids with carbon chain length between C-1 to C-14 or their ester derivatives demonstrated FIC indices of less than 1 for multiple dose combinations with various antifungal agents (different classes). Hence, the combination effect for each of the tested agents was termed synergistic against different filamentous fungi and yeasts (Table 1 to Table 14).

TABLE 1
Representative data from checkerboard assays of combination of caprylic acid with
various antifungals (various classes) on Trichophyton rubrum (ATCC-28188)

| | | Agent | Combination | | FIC | |
|---|---|---|---|---|---|---|
| Combination | Agents | MIC (μg/ml) | MIC (μg/ml) | FIC | index | Interaction |
| 1. | Clotrimazole | 0.6 | 0.075 | 0.125 | 0.375 | Synergistic |
| | (class azole) | | | | | |
| | Caprylic acid | 15.625 | 3.906 | 0.25 | | |
| 2. | Ketoconazole | 0.3125 | 0.0781 | 0.25 | 0.5 | Synergistic |
| | (class azole) | | | | | |
| | Caprylic acid | 15.625 | 3.906 | 0.25 | | |
| 3. | Efinaconazole | 0.125 | 0.0625 | 0.5 | 0.625 | Synergistic |
| | (class azole) | | | | | |
| | Caprylic acid | 15.625 | 1.95 | 0.125 | | |
| 4. | Zinc pyrithione | 3.125 | 0.195 | 0.06 | 0.19 | Synergistic |
| | Caprylic acid | 7.81 | 0.976 | 0.125 | | |
| 5. | Itraconazole | 0.125 | 0.0312 | 0.25 | 0.5 | Synergistic |
| | Caprylic acid | 15.625 | 3.906 | 0.25 | | |
| 6. | Caspofungin | 64 | 16 | 0.25 | 0.75 | Synergistic |
| | Caprylic acid | 15.625 | 7.8 | 0.5 | | |
| 7. | Piroctone olamine | 4 | 2 | 0.5 | 0.75 | Synergistic |
| | Caprylic acid | 15.625 | 3.9 | 0.25 | | |
| 8. | Ciclopirox | 2 | 0.5 | 0.25 | 0.75 | Synergistic |
| | Caprylic acid | 15.6 | 7.8 | 0.5 | | |

TABLE 2
Representative data from checkerboard assays of combination of propylene
glycol monocaprylate (caprylic acid ester) with various antifungals
(various classes) on Trichophyton rubrum (ATCC-28188)

| | | Agent | Combination | | FIC | |
|---|---|---|---|---|---|---|
| Combination | Agents | MIC (μg/ml) | MIC (μg/ml) | FIC | index | Interaction |
| 1. | Clotrimazole | 0.6 | 0.15 | 0.25 | 0.5 | Synergistic |
| | (class azole) | | | | | |
| | Propylene glycol | 125 | 31.25 | 0.25 | | |
| | monocaprylate | | | | | |
| 2. | Clotrimazole | 0.6 | 0.002 | 0.003 | 0.503 | Synergistic |
| | (class azole) | | | | | |
| | Propylene glycol | 125 | 62.5 | 0.5 | | |
| | monocaprylate | | | | | |
| 3. | Ketoconazole | 0.3 | 0.0375 | 0.125 | 0.625 | Synergistic |
| | (class azole) | | | | | |
| | Propylene glycol | 62.5 | 31.25 | 0.5 | | |
| | monocaprylate | | | | | |
| 4. | Efinaconazole | 0.125 | 0.007 | 0.056 | 0.556 | Synergistic |
| | (class azole) | | | | | |
| | Propylene glycol | 62.5 | 31.25 | 0.5 | | |
| | monocaprylate | | | | | |
| 5. | Piroctone olamine | 2.5 | 0.312 | 0.125 | 0.625 | Synergistic |
| | Propylene glycol | 125 | 62.5 | 0.5 | | |
| | monocaprylate | | | | | |
| 6. | Piroctone olamine | 2.5 | 1.25 | 0.5 | 0.75 | Synergistic |
| | Propylene glycol | 125 | 31.25 | 0.25 | | |
| | monocaprylate | | | | | |
| 7. | Zinc pyrithione | 3.125 | 0.78 | 0.25 | 0.75 | Synergistic |
| | Propylene glycol | 62.5 | 31.25 | 0.5 | | |
| | monocaprylate | | | | | |
| 8. | Zinc pyrithione | 3.125 | 1.56 | 0.5 | 0.75 | Synergistic |
| | Propylene glycol | 62.5 | 15.62 | 0.25 | | |
| | monocaprylate | | | | | |
| 9. | Itraconazole | 0.125 | 0.015 | 0.125 | 0.625 | Synergistic |
| | (class azole) | | | | | |
| | Propylene glycol | 125 | 62.5 | 0.5 | | |
| | monocaprylate | | | | | |
| 10. | Amphotericin B | 0.5 | 0.125 | 0.25 | 0.5 | Synergistic |
| | Propylene glycol | 250 | 62.5 | 0.25 | | |
| | monocaprylate | | | | | |
| 11. | Caspofungin | 64 | 0.125 | 0.002 | 0.252 | Synergistic |
| | Propylene glycol | 250 | 62.5 | 0.25 | | |
| | monocaprylate | | | | | |
| 12. | Ciclopirox | 2 | 0.06 | 0.03 | 0.503 | Synergistic |
| | Propylene glycol | 500 | 250 | 0.5 | | |
| | monocaprylate | | | | | |

TABLE 3
Representative data from checkerboard assays of combination of glyceryl monocaprylate
with various antifungals (various classes) on Trichophyton rubrum (ATCC 28188)

| | | Agent | Combination | | FIC | |
|---|---|---|---|---|---|---|
| Combination | Agents | MIC (μg/ml) | MIC (μg/ml) | FIC | index | Interaction |
| 1. | Clotrimazole | 0.6 | 0.075 | 0.125 | 0.625 | Synergistic |
| | (class azole) | | | | | |
| | Glyceryl | 62.5 | 31.25 | 0.5 | | |
| | monocaprylate | | | | | |
| 2. | Ketoconazole | 0.321 | 0.009 | 0.031 | 0.531 | Synergistic |
| | (class azole) | | | | | |
| | Glyceryl | 62.5 | 31.25 | 0.5 | | |
| | monocaprylate | | | | | |
| 3. | Efinaconazole | 0.125 | 0.0625 | 0.5 | 0.625 | Synergistic |
| | (class azole) | | | | | |
| | Glyceryl | 62.5 | 7.812 | 0.125 | | |
| | monocaprylate | | | | | |
| 4. | Zinc pyrithione | 3.125 | 0.195 | 0.0625 | 0.3125 | Synergistic |
| | Glyceryl | 62.5 | 15.625 | 0.25 | | |
| | monocaprylate | | | | | |
| 5. | Itraconazole | 0.125 | 0.007 | 0.0625 | 0.562 | Synergistic |
| | Glyceryl | 125 | 62.5 | 0.5 | | |
| | monocaprylate | | | | | |
| 6. | Caspofungin | 64 | 2 | 0.031 | 0.531 | Synergistic |
| | Glyceryl | 250 | 125 | 0.5 | | |
| | monocaprylate | | | | | |
| 7. | Piroctone olamine | 4 | 2 | 0.5 | 0.562 | Synergistic |
| | Glyceryl | 250 | 15.6 | 0.062 | | |
| | monocaprylate | | | | | |
| 8. | Ciclopirox | 2 | 0.03 | 0.015 | 0.501 | Synergistic |
| | Glyceryl | 250 | 125 | 0.5 | | |
| | monocaprylate | | | | | |

TABLE 4
Representative data from checkerboard assays of combination
of undecylenic acid (C11) with various known antifungals (various
classes) on Trichophyton rubrum (ATCC-28188).

| | | Agent | Combination | | FIC | |
|---|---|---|---|---|---|---|
| Combination | Agents | MIC (μg/ml) | MIC (μg/ml) | FIC | index | Interaction |
| 1. | Itraconazole | 0.125 | 0.0312 | 0.25 | 0.75 | Synergistic |
| | Undecylenic acid | 31.3 | 15.6 | 0.5 | | |
| 2. | Efinaconazole | 0.03 | 0.015 | 0.5 | 0.74 | Synergistic |
| | (class azole) | | | | | |
| | Undecylenic acid | 31.3 | 7.8 | 0.249 | | |
| 3. | Amphotericin B | 0.5 | 0.06 | 0.12 | 0.62 | Synergistic |
| | (Polyene) | | | | | |
| | Undecylenic acid | 62.5 | 31.3 | 0.5 | | |
| 4. | Caspofungin | 64 | 16 | 0.25 | 0.5 | Synergistic |
| | (Echinocandin) | | | | | |
| | Undecylenic acid | 62.5 | 15.6 | 0.25 | | |

TABLE 5
Representative data from checkerboard assays of combination of lauric acid (C12)
with various antifungals (various classes) on Trichophyton rubrum (ATCC-28188)

| | | Agent | Combination | | FIC | |
|---|---|---|---|---|---|---|
| Combination | Agents | MIC (μg/ml) | MIC (μg/ml) | FIC | index | Interaction |
| 1. | Itraconazole | 0.125 | 0.06 | 0.5 | 0.75 | Synergistic |
| | (Class azole) | | | | | |
| | Lauric acid | 15.6 | 3.9 | 0.25 | | |
| 2. | Efinaconazole | 0.03 | 0.015 | 0.5 | 0.75 | Synergistic |
| | (class azole) | | | | | |
| | Lauric acid | 15.6 | 3.9 | 0.25 | | |
| 3. | Clotrimazole | 0.5 | 0.125 | 0.25 | 0.75 | Synergistic |
| | (class azole) | | | | | |
| | Lauric acid | 15.6 | 7.8 | 0.5 | | |
| 4. | Amphotericin B | 0.5 | 0.125 | 0.25 | 0.75 | Synergistic |
| | (Class polyene) | | | | | |
| | Lauric acid | 15.6 | 7.8 | 0.5 | | |

TABLE 6
Representative data from checkerboard assays of combination of lauric
acid ester (propylene glycol monolaurate) with various antifungals
(various classes) on Trichophyton rubrum (ATCC-28188)

| | | Agent | Combination | | FIC | |
|---|---|---|---|---|---|---|
| Combination | Agents | MIC (μg/ml) | MIC (μg/ml) | FIC | index | Interaction |
| 1. | Itraconazole | 0.125 | 0.06 | 0.48 | 0.6 | Synergistic |
| | (Class azole) | | | | | |
| | Propylene glycol | 125 | 15.6 | 0.124 | | |
| | monolaurate | | | | | |
| 2. | Efinaconazole | 0.03 | 0.008 | 0.266 | 0.76 | Synergistic |
| | (class azole) | | | | | |
| | Propylene glycol | 125 | 62.5 | 0.5 | | |
| | monolaurate | | | | | |
| 3. | Clotrimazole | 0.5 | 0.125 | 0.25 | 0.5 | Synergistic |
| | (class azole) | | | | | |
| | Propylene glycol | 125 | 31.3 | 0.25 | | |
| | monolaurate | | | | | |
| 4. | Amphotericin B | 0.5 | 0.125 | 0.25 | 0.5 | Synergistic |
| | (Class polyene) | | | | | |
| | Propylene glycol | 125 | 31.3 | 0.25 | | |
| | monolaurate | | | | | |
| 5. | Caspofungin | 64 | 0.125 | 0.002 | 0.502 | Synergistic |
| | (Class Echinocandin) | | | | | |
| | Propylene glycol | 125 | 62.5 | 0.5 | | |
| | monolaurate | | | | | |

TABLE 7
Representative data showing synergistic action of terbinafine or butenafine with caprylic
acid (C8), propylene glycol monocaprylate (C8 ester) and glyceryl monocaprylate (C8 ester)
against terbinafine resistant Trichophyton interdigitale (GTB-2S)

| | | Agent | Combination | | FIC | |
|---|---|---|---|---|---|---|
| Combination | Agents | MIC (μg/ml) | MIC (μg/ml) | FIC | index | Interaction |
| 1. | Terbinafine | 15 | 0.117 | 0.0078 | 0.5007 | Synergistic |
| | (class allylamine) | | | | | |
| | Caprylic acid | 15.625 | 7.812 | 0.5 | | |
| 2. | Terbinafine | 30 | 7.5 | 0.3 | 0.5 | Synergistic |
| | (class allylamine) | | | | | |
| | Propylene glycol | 125 | 31.25 | 0.25 | | |
| | monocaprylate | | | | | |
| 3. | Terbinafine | 30 | 0.117 | 0.004 | 0.504 | Synergistic |
| | (class allylamine) | | | | | |
| | Propylene glycol | 125 | 62.5 | 0.5 | | |
| | monocaprylate | | | | | |
| 4. | Terbinafine | 15 | 0.468 | 0.0312 | 0.531 | Synergistic |
| | (class allylamine) | | | | | |
| | Glyceryl | 15.625 | 7.812 | 0.5 | | |
| | monocaprylate | | | | | |
| 5. | Butenafine | 7.5 | 0.468 | 0.0625 | 0.562 | Synergistic |
| | (class benzylamine) | | | | | |
| | Caprylic acid | 15.625 | 7.812 | 0.5 | | |
| 6. | Butenafine | 7.5 | 0.117 | 0.016 | 0.516 | Synergistic |
| | (class benzylamine) | | | | | |
| | Propylene glycol | 125 | 62.5 | 0.5 | | |
| | monocaprylate | | | | | |
| 7. | Butenafine | 7.5 | 0.234 | 0.0312 | 0.531 | Synergistic |
| | (class benzylamine) | | | | | |
| | Glyceryl | 15.625 | 7.812 | 0.5 | | |
| | monocaprylate | | | | | |

TABLE 8
Representative data from checkerboard assays of combination of luliconazole (azole
class) with caprylic acid or glyceryl monocaprylate on C. albicans (ATCC-90028)

| | | Agent | Combination | | FIC | |
|---|---|---|---|---|---|---|
| Combination | Agents | MIC (μg/ml) | MIC (μg/ml) | FIC | index | Interaction |
| 1. | Luliconazole | 7.5 | 0.117 | 0.015 | 0.265 | Synergistic |
| | (class azoles) | | | | | |
| | Caprylic acid | 125 | 31.25 | 0.25 | | |
| 2. | Luliconazole | 7.5 | 0.117 | 0.0156 | 0.0781 | Synergistic |
| | (class azoles) | | | | | |
| | Glyceryl | 500 | 31.25 | 0.0625 | | |
| | monocaprylate | | | | | |

TABLE 9
Representative data from checkerboard assays of combination of caprylic acid
(C8) with various antifungals on azole resistant C. albicans MTCC 227

| | | Agent | Combination | | FIC | |
|---|---|---|---|---|---|---|
| Combination | Agents | MIC (μg/ml) | MIC (μg/ml) | FIC | index | Interaction |
| 1. | Clotrimazole | 8 | 0.25 | 0.031 | 0.093 | Synergistic |
| | (class azoles) | | | | | |
| | Caprylic acid | 250 | 15.62 | 0.062 | | |
| 2. | Luliconazole | 8 | 0.5 | 0.062 | 0.3 | Synergistic |
| | (class azoles) | | | | | |
| | Caprylic acid | 250 | 62.5 | 0.25 | | |
| 3. | Efinaconazole | 128 | 0.5 | 0.062 | 0.3 | Synergistic |
| | (class azoles) | | | | | |
| | Caprylic acid | 250 | 62.5 | 0.25 | | |
| 4. | Ketoconazole | 64 | 0.25 | 0.003 | 0.253 | Synergistic |
| | (class azoles) | | | | | |
| | Caprylic acid | 250 | 62.5 | 0.25 | | |
| 5. | Itraconazole | 32 | 4 | 0.125 | 0.25 | Synergistic |
| | (class azoles) | | | | | |
| | Caprylic acid | 250 | 31.25 | 0.125 | | |

TABLE 10
Representative data from checkerboard assays of combination
of propylene glycol monocaprylate (C8 ester) with various known
antifungals on azole resistant C. albicans MTCC 227

| | | Agent | Combination | | FIC | |
|---|---|---|---|---|---|---|
| Combination | Agents | MIC (μg/ml) | MIC (μg/ml) | FIC | index | Interaction |
| 1. | Clotrimazole | 16 | 4 | 0.25 | 0.312 | Synergistic |
| | (class azoles) | | | | | |
| | Propylene glycol | 500 | 31.25 | 0.0625 | | |
| | monocaprylate | | | | | |
| 2. | Luliconazole | 8 | 0.5 | 0.0625 | 0.187 | Synergistic |
| | (class azoles) | | | | | |
| | Propylene glycol | 500 | 62.5 | 0.125 | | |
| | monocaprylate | | | | | |
| 3. | Efinaconazole | 128 | 4 | 0.031 | 0.281 | Synergistic |
| | (class azoles) | | | | | |
| | Propylene glycol | 500 | 125 | 0.25 | | |
| | monocaprylate | | | | | |
| 4. | Ketoconazole | 128 | 8 | 0.0625 | 0.187 | Synergistic |
| | (class azoles) | | | | | |
| | Propylene glycol | 500 | 62.5 | 0.125 | | |
| | monocaprylate | | | | | |
| 5. | Itraconazole | 64 | 0.125 | 0.002 | 0.064 | Synergistic |
| | (class azoles) | | | | | |
| | Propylene glycol | 500 | 31.25 | 0.062 | | |
| | monocaprylate | | | | | |

TABLE 11
Representative data from checkerboard assays of combination of glyceryl monocaprylate
(C8 ester) with various antifungals on azole resistant C. albicans MTCC 227

| | | Agent | Combination | | FIC | |
|---|---|---|---|---|---|---|
| Combination | Agents | MIC (μg/ml) | MIC (μg/ml) | FIC | index | Interaction |
| 1. | Clotrimazole | 16 | 4 | 0.25 | 0.5 | Synergistic |
| | (class azoles) | | | | | |
| | Glyceryl | 1250 | 312.5 | 0.25 | | |
| | monocaprylate | | | | | |
| 2. | Efinaconazole | 128 | 1 | 0.007 | 0.257 | Synergistic |
| | (class azoles) | | | | | |
| | Glyceryl | 1250 | 312.5 | 0.25 | | |
| | monocaprylate | | | | | |
| 3. | Ketoconazole | 128 | 2 | 0.0156 | 0.265 | Synergistic |
| | (class azoles) | | | | | |
| | Glyceryl | 1250 | 312.5 | 0.25 | | |
| | monocaprylate | | | | | |
| 4. | Itraconazole | 32 | 4 | 0.125 | 0.375 | Synergistic |
| | (class azoles) | | | | | |
| | Glyceryl | 1250 | 312.5 | 0.25 | | |
| | monocaprylate | | | | | |

TABLE 12
Representative data from checkerboard assays of combination of undecylenic acid
(C11) with various antifungals on azole resistant C. albicans MTCC 227

| | | Agent | Combination | | FIC | |
|---|---|---|---|---|---|---|
| Combination | Agents | MIC (μg/ml) | MIC (μg/ml) | FIC | index | Interaction |
| 1. | Clotrimazole | 16 | 2 | 0.125 | 0.187 | Synergistic |
| | (class azoles) | | | | | |
| | Undecylenic acid | 500 | 31.25 | 0.0625 | | |
| 2. | Luliconazole | 8 | 1 | 0.125 | 0.187 | Synergistic |
| | (class azoles) | | | | | |
| | Undecylenic acid | 500 | 31.25 | 0.0625 | | |
| 3. | Efinaconazole | 128 | 4 | 0.031 | 0.093 | Synergistic |
| | (class azoles) | | | | | |
| | Undecylenic acid | 500 | 31.25 | 0.062 | | |
| 4. | Ketoconazole | 128 | 1 | 0.0078 | 0.132 | Synergistic |
| | (class azoles) | | | | | |
| | Undecylenic acid | 500 | 62.5 | 0.125 | | |
| 5. | Itraconazole | 32 | 0.125 | 0.003 | 0.066 | Synergistic |
| | (class azoles) | | | | | |
| | Undecylenic acid | 500 | 31.25 | 0.06 | | |

TABLE 13
Representative data from checkerboard assays of combination of lauric acid
(C12) with various antifungals on azole resistant C. albicans MTCC 227

| | | Agent | Combination | | FIC | |
|---|---|---|---|---|---|---|
| Combination | Agents | MIC (μg/ml) | MIC (μg/ml) | FIC | index | Interaction |
| 1. | Clotrimazole | 8 | 1 | 0.125 | 0.187 | Synergistic |
| | (class azoles) | | | | | |
| | Lauric acid | 3125 | 195.3 | 0.062 | | |
| 2. | Luliconazole | 8 | 1 | 0.125 | 0.25 | Synergistic |
| | (class azoles) | | | | | |
| | Lauric acid | 3125 | 390.6 | 0.125 | | |
| 3. | Efinaconazole | 128 | 0.5 | 0.003 | 0.128 | Synergistic |
| | (class azoles) | | | | | |
| | Lauric acid | 3125 | 390.6 | 0.125 | | |
| 4. | Ketoconazole | 64 | 0.25 | 0.003 | 0.127 | Synergistic |
| | (class azoles) | | | | | |
| | Lauric acid | 3125 | 390.6 | 0.124 | | |
| 5. | Itraconazole | 32 | 0.125 | 0.003 | 0.253 | Synergistic |
| | (class azoles) | | | | | |
| | Lauric acid | 3125 | 781.2 | 0.25 | | |

TABLE 14
Representative data from checkerboard assays of combination
of propylene glycol monolaurate (C12 ester ) with various known
antifungals on azole resistant C. albicans MTCC 227

| | | Agent | Combination | | FIC | |
|---|---|---|---|---|---|---|
| Combination | Agents | MIC (μg/ml) | MIC (μg/ml) | FIC | index | Interaction |
| 1. | Clotrimazole | 16 | 8 | 0.5 | 0.515 | Synergistic |
| | (class azoles) | | | | | |
| | Propylene glycol | 250000 | 3906 | 0.015 | | |
| | monolaurate | | | | | |
| 2. | Efinaconazole | 128 | 0.5 | 0.003 | 0.018 | Synergistic |
| | (class azoles) | | | | | |
| | Propylene glycol | 250000 | 3906 | 0.015 | | |
| | monolaurate | | | | | |
| 3. | Ketoconazole | 128 | 32 | 0.25 | 0.281 | Synergistic |
| | (class azoles) | | | | | |
| | Propylene glycol | 250000 | 7812 | 0.0312 | | |
| | monolaurate | | | | | |
| 4. | Itraconazole | 32 | 2 | 0.0625 | 0.07 | Synergistic |
| | (class azoles) | | | | | |
| | Propylene glycol | 250000 | 3906 | 0.015 | | |
| | monolaurate | | | | | |

Thus, the above results show that the medium chain fatty acids of C-1 to C-14 carbon chain length, such as between C-11 to C-14 or C-1 to C-10 and their esters demonstrate synergistic behavior with all the tested antifungal agents. ‘Table 1’ shows representative data from checkerboard assays of combination of caprylic acid with various antifungals (various classes) on Trichophyton rubrum (ATCC 28188). ‘Table 2’ shows representative data from checkerboard assays of combination of propylene glycol monocaprylate with various antifungals (various classes) on Trichophyton rubrum (ATCC 28188). ‘Table 3’ shows representative data from checkerboard assays of combination of glyceryl monocaprylate with various antifungals (various classes) on Trichophyton rubrum (ATCC 28188). ‘Table 4’ shows representative data from checkerboard assays of combination of undecylenic acid (C11) with various antifungals (various classes) on Trichophyton rubrum (ATCC 28188). ‘Table 5’ shows representative data from checkerboard assays of combination of lauric acid (C12) with various antifungals (various classes) on Trichophyton rubrum (ATCC 28188). ‘Table 6’ shows representative data from checkerboard assays of combination of propylene glycol monolaurate (lauric acid ester) with various antifungals (various classes) on Trichophyton rubrum (ATCC 28188). ‘Table 7’ shows representative data of synergistic action of terbinafine or butenafine with caprylic acid, propylene glycol monocaprylate and glyceryl monocaprylate against terbinafine resistant Trichophyton interdigitale (GTB-2S). ‘Table 8’ shows representative synergistic combinations of luliconazole with caprylic acid or its ester (glyceryl monocaprylate) on C. albicans (ATCC 90028). ‘Table 9’ further shows representative synergistic combinations of caprylic acid (C8) with various antifungals on azole resistant C. albicans MTCC 227. Propylene glycol monocaprylate (an ester of caprylic acid) also shows synergistic action with various known antifungals on azole resistant C. albicans MTCC 227 (‘Table 10’). Glyceryl monocaprylate (another caprylic acid ester) also shows synergistic action with various antifungals on azole resistant C. albicans MTCC 227 as shown in ‘Table 11’. Representative data from checkerboard assays of combination of undecylenic acid (C11) with various antifungals on azole resistant C. albicans MTCC 227 clearly demonstrates synergistic antifungal properties (‘Table 12’). ‘Table 13’ shows representative synergistic data from checkerboard assays of combination of lauric acid (C12) with various known antifungals on azole resistant C. albicans MTCC 227. Further, an ester of lauric acid, propylene glycol monolaurate also demonstrates synergistic action on azole resistant C. albicans MTCC 227 when in combination with various known antifungals (‘Table 14’).

Taken together, these data reveal that the investigational medium-chain fatty acids of C-1 to C-14 carbon chain length and their esters demonstrate synergistic behavior with all the tested antifungal agents against various fungal species. Further, only an exhaustive checkerboard assay system allowed identification of synergistic combination properties for combination of medium-chain fatty acids with a range of antifungal agents. It is noteworthy that only representative combinations are tabulated under Example 1; however, synergy was observed for multiple dose ranges for each of pair of agents.

On the contrary, when the same checkerboard method was used to test for any combination activity between the antifungal agent of azole class such as clotrimazole, and fatty acid having carbon chain length or C-15 or above, such as oleic acid (C18) against C. albicans, no synergy was observed. Instead, an antagonistic effect was clearly seen as depicted in FIGS. 1B and 1C.

### Example 2: Preparation of Various Oil Compositions Containing Piroctone Olamine and Caprylic Acid

The compositions were prepared by dissolving the active agent in ethanol or isopropyl alcohol (IPA). The oleyl alcohol was then added and stirred until a homogenous solution was obtained. Other excipients or additives were added and stirred to get clear solution except liquid paraffin. Weight was finally made up with liquid paraffin and stirred, until homogenous solution was obtained. Final formulations were clear transparent oil solutions. ‘Table 15’ describes anti-fungal clear oil compositions containing piroctone olamine as anti-fungal agent and medium chain fatty acid and/or esters using various excipients or additives.

TABLE 15
Piroctone olamine - Caprylic Acid - oil compositions

| | | | | | | Cap. | Toco. | | Cyclo- | LLP | | |
|---|---|---|---|---|---|---|---|---|---|---|---|---|
| Formula | PO | Ethanol | IPA | OA | Triacetin | A | Ace. | TTO | methicone | ml, | | |
| Code | (mg) | (ml) | (ml) | (ml) | (ml) | (ml) | (mg) | (ml) | (ml) | upto | pH | App |
| VPO-001 | 50 | 0.4 | — | 0.4 | — | 0.1 | — | 0.5 | — | 10 | 6-7 | C |
| VPO-002 | 50 | 0.4 | — | 0.4 | — | 0.1 | — | — | — | 10 | 6-7 | C |
| VPO-018 | 10 | — | 0.2 | 0.2 | — | 0.02 | — | — | — | 10 | 6-7 | C |
| VPO-019 | 10 | — | 0.2 | 0.2 | 0.02 | 0.02 | — | — | — | 10 | 6-7 | C |
| VPO-020 | 10 | — | 0.2 | 0.3 | 0.04 | 0.02 | — | — | — | 10 | 6-7 | C |
| VPO-021 | 10 | — | 0.2 | 0.5 | 0.06 | 0.02 | — | — | — | 10 | 6-7 | C |
| VPO-022 | 10 | — | — | 0.4 | — | 0.02 | — | — | — | 10 | 6-7 | C |
| VPO-023 | 10 | — | — | 0.4 | 0.02 | 0.02 | — | — | — | 10 | 6-7 | C |
| VPO-024 | 10 | — | — | 0.5 | 0.04 | 0.02 | — | — | — | 10 | 6-7 | C |
| VPO-025 | 10 | 0.05 | — | 0.05 | — | 0.02 | — | — | — | 10 | 6-7 | ST |
| VPO-026 | 10 | 0.05 | — | 0.3 | 0.02 | 0.02 | — | — | — | 10 | 6-7 | C |
| VPO-027 | 10 | 0.05 | — | 0.3 | 0.02 | 0.02 | — | 0.1 | — | 10 | 6-7 | C |
| VPO-028 | 10 | 0.05 | — | 0.1 | — | 0.02 | — | — | — | 10 | 6-7 | C |
| VPO-030 | 10 | — | 0.2 | 0.2 | — | 0.02 | — | — | 0.1 | 10 | 6-7 | C |
| VPO-031 | 10 | 0.05 | — | 0.1 | — | 0.02 | — | — | 0.1 | 10 | 6-7 | C |
| VPO-032 | 10 | — | 0.2 | 0.2 | — | 0.02 | — | — | 1.0 | 10 | 6-7 | C |
| VPO-033 | 10 | 0.05 | — | 0.1 | — | 0.02 | — | — | 1.0 | 10 | 6-7 | C |
| VPO-034 | 10 | — | 0.2 | 0.2 | — | 0.02 | 50 | — | — | 10 | 6-7 | C |
| VPO-035 | 10 | 0.05 | — | 0.1 | — | 0.02 | 100 | — | — | 10 | 6-7 | C |
| VPO-036 | 10 | — | 0.2 | 0.2 | — | 0.02 | 25 | — | — | 10 | 6-7 | C |
| VPO-037 | 10 | — | 0.2 | 0.2 | — | 0.02 | 50 | — | — | 10 | 6-7 | C |
| VPO-038 | 10 | — | 0.2 | 0.2 | — | 0.02 | 75 | — | — | 10 | 6-7 | C |
| VPO-039 | 10 | — | 0.2 | 0.2 | — | 0.02 | 100 | — | — | 10 | 6-7 | C |
| VPO-040 | 10 | — | 0.2 | 0.2 | — | 0.02 | 100 | — | 1.0 | 10 | 6-7 | C |
| VPO-041 | 10 | — | 0.2 | 0.2 | — | 0.02 | 100 | 0.2 | 1.0 | 10 | 6-7 | C |
| VPO-042 | 10 | 0.05 | — | 0.1 | — | 0.02 | 25 | — | — | 10 | 6-7 | C |
| VPO-043 | 10 | 0.05 | — | 0.1 | — | 0.02 | 50 | — | — | 10 | 6-7 | C |
| VPO-044 | 10 | 0.05 | — | 0.1 | — | 0.02 | 75 | — | — | 10 | 6-7 | C |
| VPO-045 | 10 | 0.05 | — | 0.1 | — | 0.02 | 100 | — | — | 10 | 6-7 | C |
| VPO-046 | 10 | 0.05 | — | 0.1 | — | 0.02 | 100 | — | 1.0 | 10 | 6-7 | C |
| VPO-047 | 10 | 0.05 | — | 0.1 | — | 0.02 | 100 | 0.2 | 1.0 | 10 | 6-7 | C |
| VPO-049 | 10 | — | — | — | — | 0.3 | — | — | — | 10 | 6-7 | C |
| VPO-050 | 10 | — | — | 0.2 | — | 0.2 | — | — | — | 10 | 6-7 | C |
| VPO-051 | 10 | — | — | — | — | 0.3 | 50 | 0.1 | 0.5 | 10 | 6-7 | C |
| VPO-052 | 10 | — | — | 0.1 | — | 0.3 | 50 | 0.2 | 0.5 | 10 | 6-7 | C |

C—Clear,
ST—Slight turbid,
PO—Piroctone olamine,
IPA—Isopropyl alcohol,
OA—oleyl alcohol,
Cap. A—Caprylic acid,
Toco. Ace.—Tocopherol acetate,
TTO—tea tree oil,
LLP—light liquid paraffin,
App—appearance

Result:
1. Compositions using liquid paraffin as base oil (excipient) containing piroctone olamine, were clear oil solutions.
2. Addition of other excipients such as tea tree oil, cyclomethicone (D₄), tocopherol acetate etc. did not affect the physical stability of formulations as compositions appeared as clear oil solutions.

### Example 3: Preparation of Various Oil Compositions Containing Ketoconazole and Caprylic Acid

The compositions were prepared by dissolving the active agent in ethanol. The oleyl alcohol was then added and stirred until homogenous solution was obtained. Other excipients or additives were added and stirred to get clear solution except liquid paraffin. Weight was finally made up with liquid paraffin and stirred until homogenous solution was obtained. Final formulations were clear transparent oil solutions. ‘Table 16’ describes anti-fungal clear oil compositions containing ketoconazole as anti-fungal agent and medium chain fatty acid and/or esters using various excipients or additives.

TABLE 16
Ketoconazole - Caprylic Acid - Oil compositions

| | | | Oleyl | Caprylic | Tea Tree | Terpene- | Tocopherol | Cyclo- | Liq. | | |
|---|---|---|---|---|---|---|---|---|---|---|---|
| Formulation | Keto | Ethanol | Alco | acid | Oil | 4-0l | acetate | methicone | Paraffin | | |
| Code | (mg) | (ml) | (ml) | (ml) | (ml) | (ml) | (mg) | (ml) | (ml, upto) | pH | App. |
| VK-001 | 10 | 0.5 | 0.45 | — | 0.5 | — | — | — | 10 | 6-7 | C |
| VK-002 | 10 | 0.5 | 0.45 | — | — | — | — | — | 10 | 6-7 | C |
| VK-012 | 5 | 0.3 | 0.3 | — | — | — | — | — | 10 | 6-7 | C |
| VK-013 | 5 | 0.3 | 0.3 | — | 0.1 | — | — | — | 10 | 6-7 | C |
| VK-014 | 5 | 0.3 | 0.3 | — | 0.2 | — | — | — | 10 | 6-7 | C |
| VK-015 | 5 | 0.3 | 0.3 | — | 0.3 | — | — | — | 10 | 6-7 | C |
| VK-016 | 5 | 0.3 | 0.3 | — | 0.4 | — | — | — | 10 | 6-7 | C |
| VK-017 | 5 | 0.3 | 0.3 | — | 0.1 | — | — | 0.2 | 10 | 6-7 | C |
| VK-018 | 5 | 0.3 | 0.3 | — | 0.1 | — | — | 0.5 | 10 | 6-7 | C |
| VK-019 | 5 | 0.3 | 0.3 | — | 0.1 | — | — | 1.0 | 10 | 6-7 | C |
| VK-020 | 5 | 0.3 | 0.3 | — | — | — | — | 1.0 | 10 | 6-7 | C |
| VK-030 | 5 | — | 0.1 | 0.3 | — | 0.1 | 20 | — | 10 | 6-7 | ST |
| VK-031 | 5 | — | 0.1 | 0.3 | — | 0.2 | 20 | — | 10 | 6-7 | C |
| VK-032 | 5 | — | 0.1 | 0.3 | — | 0.3 | 20 | — | 10 | 6-7 | C |
| VK-036 | 5 | — | 0.1 | 0.4 | — | 0.1 | 20 | — | 10 | 6-7 | C |
| VK-037 | 5 | — | 0.1 | 0.4 | — | 0.2 | 20 | — | 10 | 6-7 | C |
| VK-038 | 5 | — | 0.1 | 0.4 | — | 0.3 | 20 | — | 10 | 6-7 | ST |
| VK-040 | 5 | — | 0.4 | 0.4 | — | 0.4 | 20 | — | 10 | 6-7 | C |

C—Clear transparent,
ST—Slight turbid

Result:
1. Compositions using liquid paraffin as base oil (excipient) containing ketoconazole and medium chain fatty acid and/or esters along with other excipients appeared as clear oil solutions.
2. Addition of other excipients such as tea tree oil, terpene-4-ol, cyclomethicone (D₄) etc. did not affect the physical stability of formulations and compositions appeared as clear oil solution.

### Example 4: Study of Fatty Acid/Esters Having C-15 or Greater Carbon Chain Length as a Source of Nutrients for the Growth of Malassezia spp

Malassezia species are lipophilic unipolar yeasts recognized as commensals of skin that may be pathogenic under certain conditions (Jindo et al 2004: Indian Journal of Medical Microbiology (22: 179). To compare lipid requirements of the fungus most closely associated with dandruff/seborrheic dermatitis, the best studied Malassezia species is M. furfur. Lipid assimilation in vitro assay was designed to investigate lipid effect on growth of M. furfur (MTCC 1374).

Method:

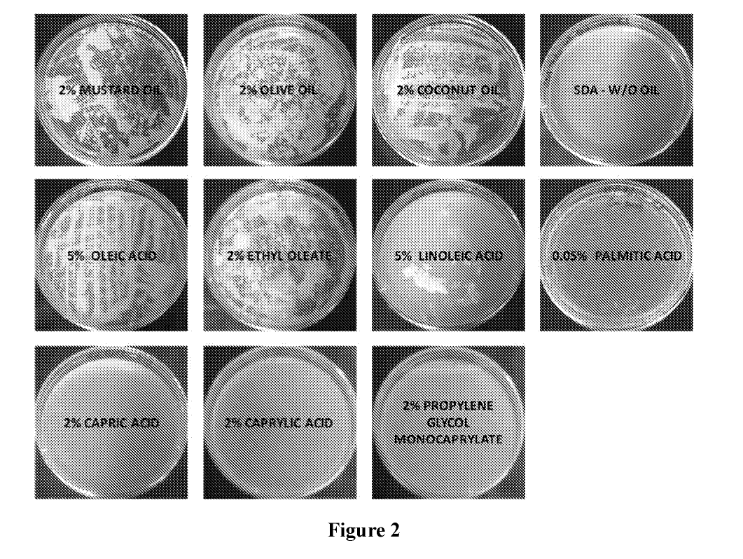

Briefly, Sabouraud Dextrose containing low-melt agar was melted, cooled to 38° C. Fatty acids/esters constituents eg, propylene glycol monocaprylate (C-8), capric acid (C-10), caprylic acid (C-8), linoleic acid (C-18), oleic acid (C-18), palmitic acid (C-16), ethyl oleate (C-18), and oils containing fatty acid/esters eg, coconut oil, mustard oil etc., were added to study the growth of the fungus (Kaw Bing CHUA, et al Malaysian J Pathol (2005) 27(2): 99). After solidification, agar plates were streaked with M. furfur innoculum adjusted to appropriate cfu/ml, aseptically. Positive control with 2% olive oil and negative control without fatty substance were also maintained. The results are provided in FIG. 2.

Results:
1. Results showed that there was no growth of M. furfur in absence of fatty acids/esters or oils in in vitro condition up to 6 days (data not shown).
2. Culture media which contained C-15 or greater fatty acids or esters eg, linoleic acid, oleic acid, palmitic acid, ethyl oleate and oils containing fatty acid/esters eg, coconut oil, mustard oil etc. showed confluent growth of fungus after 6 days.
3. Culture media with lower carbon fatty acids (C≤14) eg, propylene glycol monocaprylate, caprylic acid and capric acid failed to provide nutrient for the growth of fungus and no growth was observed even after 6 days.

The above results confirm that C-15 or greater fatty acids or their esters are not suitable in antifungal compositions since presence of the same promotes fungal growth, thereby reducing or inhibiting the antifungal effects/activity of the antifungal agent/composition. Accordingly, C-1 to C-14 based fatty acids or their esters/derivatives are suitable candidates along with antifungal agents, which further demonstrate synergistic antifungal activity as shown in this application.

### Example 5: Bioactivity of Oil Compositions Described in Example 2 Against M. furfur

The Minimum Inhibitory Concentration (MIC) is considered as an index for indicating anti-fungal efficacy. Therefore, lower the value of MIC of the composition, the better is its antifungal efficacy.

Method:

The in vitro activities of some of the oil compositions containing piroctone olamine against Malassezia furfur (MTCC 1374) were determined by agar dilution methods (Jan Faergemann, et al Acta Derm Venereol, (2006). 86:312; Irith Wiegand, et al Nature Protocols (2008), 3:163) Appropriate dilutions of solubilized antifungal compositions were added to molten Leeming Notman Medium. Once the plates were set, M. furfur innoculum adjusted to appropriate cfu/ml was streaked on the agar plates and incubated for 6 days. After incubation, the plates were observed at day 3 and day 6 for visible M. furfur growth. The MIC is defined as as the lowest concentration of antifungal agents that inhibits visible growth of fungus.

TABLE 17
Results of MIC for oil compositions of piroctone olamine-caprylic acid

| Formulation | Concentration (μg/ml) | | | |
|---|---|---|---|---|
| Codes | 8 | 16 | 32 | 64 |
| VPO-001 | + | + | — | — |
| VPO-018 | + | + | — | — |
| VPO-022 | + | + | — | — |
| VPO-028 | + | + | — | — |
| VPO-030 | + | + | — | — |
| VPO-031 | + | + | — | — |
| VPO-032 | + | + | — | — |
| VPO-033 | + | + | — | — |
| VPO-034 | + | + | — | — |
| VPO-035 | + | + | — | — |
| VPO-NC (Negative control) | + | + | + | + |
| PO in DMSO (Positive control) | + | + | — | — |

“+” indicates growth of fungus and “—” indicates no growth of fungus

Results:
1. Piroctone olamine-caprylic acid containing oil compositions VPO-018, VPO-022, and VPO-028 with different solvents isopropyl alcohol, oleyl alcohol, and ethanol, respectively, showed MIC at 32 μg/ml which is similar to the MIC of positive control where drug is dissolved in DMSO at the same concentration as shown in ‘Table 17’.

Addition of other additives such as cyclomethicone (D₄), tocopherol acetate etc. did not affect the MIC of oil compositions when used at concentrations as shown in Table 15.

### Example 6: Bioactivity of Oil Compositions Described in Example 3 Against M. furfur

The in vitro activities of some of the oil compositions containing ketoconazole against Malassezia furfur (MTCC 1374) were determined by agar dilution methods. Appropriate dilutions of antifungal compositions were added to molten Leeming Notman Medium. Once the plates were set, M. furfur innoculum adjusted to appropriate cfu/ml was streaked on the agar plates and incubated for 6 days. After incubation, the plates were observed at day 3 and day 6 for visible M. furfur growth. The MIC is defined as the lowest tested dilution of antifungal active that yields no growth.

TABLE 18
Results of MIC for oil compositions of ketoconazole
and caprylic acid against M. furfur (MTCC-1374)

| Formulation | Concentration (μg/ml) | | | | | |
|---|---|---|---|---|---|---|
| Codes | 0.0625 | 0.125 | 0.25 | 0.5 | 1 | 2 |
| VK-001 | + | + | — | — | — | — |
| VK-002 | + | + | — | — | — | — |
| VK-013 | + | + | — | — | — | — |
| VK-019 | + | + | — | — | — | — |
| VK-NC | + | + | + | + | + | + |
| (Negative control) | | | | | | |
| Keto in DMSO | + | + | — | — | — | — |
| (Positive control) | | | | | | |

“+” indicates growth of fungus and “—” indicates no growth of fungus

Result:
1. Ketoconazole containing oil compositions showed MIC at 0.25 μg/ml, which is similar to the MIC of positive control where drug is dissolved in DMSO at the same concentration as shown in ‘Table 18’.

### Example 7: Preparation of Oil Compositions Devoid of C-15 or Greater Fatty Acids/Esters and Containing Antifungal Agent or Combination Thereof

A) Preparation of Oil Compositions Devoid of C-15 or Greater Fatty Acids/Esters and Containing Piroctone Olamine as Antifungal Agent

These compositions were prepared by dissolving the active agent in ethanol or other suitable solvent. The oleyl alcohol was then added and stirred until a homogenous solution was obtained.

Other excipients or additives were added and stirred to obtain a clear solution except liquid paraffin. The total volume was finally made up with liquid paraffin and stirred until homogenous solution was obtained. Final formulations were clear transparent oil solutions and coded as 1P, 2P, 3P and 4P as given in ‘Table 19’. All compositions are clear transparent solutions. In compositions 1P and 2P, caprylic acid was added to balance the pH of the formulations.

TABLE 9
Oil compositions containing piroctone olamine as antifungal
agent and caprylic acid or and/or its ester

| | Compositions | | | |
|---|---|---|---|---|
| Ingredients | 1 P | 2 P | 3 P | 4 P |
| Piroctone olamine (mg) | 50 | 50 | 50 | 50 |
| Oleyl alcohol (ml) | 0.5 | 0.5 | 2 | 3 |
| Ethanol (ml) | 0.3 | 0.3 | 0.75 | 0.75 |
| Caprylic acid (ml) | 0.08 | 0.08 | — | — |
| Propylene glycol mono | — | — | 4 | 4 |
| caprylate (ml) | | | | |
| Tocopherol acetate | q.s.* | q.s. | q.s. | q.s. |
| (antioxidant) | | | | |
| Butylated hydroxy toluene | q.s. | q.s. | q.s. | q.s. |
| (Preservative) | | | | |
| Light liquid paraffin | 100 | 100 | 100 | 100 |
| (ml, Up to) | | | | |

*q.s. quantity sufficient

B) Study of MIC of Oil Compositions Devoid of C-15 or Greater Fatty Acids/Esters Containing Antifungal Agent Piroctone Olamine Against Malassezia Spp. Under In Vitro Conditions

As shown in ‘Table 20’ and ‘Table 21’, oil compositions containing piroctone olamine devoid of C-15 or greater fatty acids or their esters showed MIC in the range of 16-32 μg/ml against M. furfur (MTCC 1374) and in the range of 8-16 μg/ml against M. obtusa (CBS 7876). Composition having similar amount of piroctone olamine with 5% sunflower oil and 10% oleic acid were showed MIC at 64 μg/ml against both the strains. These results show that the presence of vegetable oil (sunflower) which is rich in triglycerides/free fatty acids especially above C-14, has an adverse effect on the activity of the antifungal agent. Similarly, the presence of fatty acids above C-14 (such as oleic acid C-18) also has an adverse effect on the activity of the antifungal agent.

TABLE 20
MIC of oil compositions containing piroctone olarnine and
caprylic acid and/or its ester against M. furfur (MTCC 1374)

| | concentration (μg/ml) | | | |
|---|---|---|---|---|
| Compositions | 8 | 16 | 32 | 64 |
| 1 P | + | + | — | — |
| 2 P | + | + | — | — |
| 3 P | + | — | — | — |
| 4 P | + | — | — | — |
| Base formulation with 5% sunflower oil | + | + | + | — |
| Base formulation with 10% oleic acid | + | + | + | — |
| Piroctone olamine in DMSO (Positive | + | + | — | — |
| control) | | | | |

“+” indicates growth of fungus and “—” indicates no growth of fungus

TABLE 21
MIC for oil compositions containing piroctone olamine and
caplic acid and/or its ester against M. obtusa (CBS 7876)

| | Concentration (μg/ml) | | | | |
|---|---|---|---|---|---|
| Compositions | 4 | 8 | 16 | 32 | 64 |
| 1 P | + | + | — | — | — |
| 2 P | + | + | — | — | — |
| 3 P | + | — | — | — | — |
| 4 P | + | — | — | — | — |
| Base formulation with 5% | + | + | + | + | — |
| sunflower oil | | | | | |
| Base formulation with 10% | + | + | + | + | — |
| oleic acid | | | | | |
| Piroctone olamine in DMSO | + | + | + | — | — |
| (positive control) | | | | | |

“+” indicates growth of fungus and “—” indicates no growth of fungus

C) Preparation of Oil Compositions Devoid of C-15 or Greater Fatty Acids/Esters Containing Ketoconazole as Antifungal Agent

These compositions were prepared by dissolving the active agent in ethanol or other suitable solvent. The oleyl alcohol was then added and stirred until a homogenous solution was obtained. Other excipients or additives were added and stirred to obtain a clear solution except liquid paraffin. The total volume was finally made up with liquid paraffin and stirred until homogenous solution was obtained. Final formulations were clear transparent oil solutions and coded as 1K, 2K, as given in ‘Table 22’. All compositions are clear transparent solutions.

TABLE 22
Oil compositions containing ketoconazole as antifungal
agent and caprylic acid and/or its ester

| | Compositions | |
|---|---|---|
| Ingredients | 1 K | 2 K |
| Ketoconazole (mg) | 70 | 20 |
| Oleyl alcohol (ml) | 0.5 | 0.5 |
| Ethanol (ml) | 0.3 | 0.1 |
| Caprylic acid (ml) | 0.5 | 0.7 |
| Propylene glycol mono caprylate (ml) | 4 | 4 |
| Tocopherol acetate (antioxidant) | q.s. | q.s. |
| Butylated hydroxy toluene (Preservative) | q.s. | q.s. |
| Light liquid paraffin (ml, Up to) | 100 | 100 |

*q.s. Quantity sufficient

D) Preparation of Oil Compositions Devoid of C-15 or Greater Fatty Acids/Esters Containing Piroctone Olamine and Ketoconazole as Antifungal Agents in Combination

These compositions were prepared by dissolving the active agent in ethanol or other suitable solvent. The oleyl alcohol was then added and stirred until a homogenous solution was obtained. Other excipients or additives were added and stirred to obtain a clear solution except liquid paraffin. The total volume was finally made up with liquid paraffin and stirred until homogenous solution was obtained. Final formulations were clear transparent oil solutions and coded as 1 PK, 2PK, as given in ‘Table 23’.

TABLE 23
Oil compositions containing piroctone olamine and ketoconazole
in combination and caprylic acid and/or its ester

| | Compositions | |
|---|---|---|
| Ingredients | 1 PK | 2 PK |
| Piroctone olamine (mg) | 50 | 50 |
| Ketoconazole (mg) | 20 | 20 |
| Oleyl alcohol (ml) | 2 | 2 |
| Ethanol (ml) | 1 | 1 |
| Caprylic acid (ml) | 0.1 | 0.1 |
| Propylene glycol mono caprylate (ml) | — | 4 |
| Tocopherol acetate (antioxidant) | q.s. | q.s. |
| Butylated hydroxy toluene (Preservative) | q.s. | q.s. |
| Light liquid paraffin (ml, Up to) | 100 | 100 |

*q.s. Quantity sufficient

E) Preparation of Oil Compositions Devoid of C-15 or Greater Fatty Acids/Esters Containing Antifungal Agent with Hair Growth Promoter (Minoxidil)

The compositions were prepared as described above (Example 6, D) and coded as 1PM, 2PM and 3PM. as given in ‘Table 24’.

TABLE 24
Oil compositions containing piroctone olamine as antifungal
agent, minoxidil and caprylic acid and/or its ester

| | Compositions | | |
|---|---|---|---|
| Ingredients | 1 PM | 2 PM | 3 PM |
| Minoxidil (mg) | 500 | 500 | 500 |
| Piroctone olamine (mg) | 50 | 50 | — |
| Ethanol (ml) | 10 | 15 | 20 |
| Propylene glycol | — | — | 10 |
| Oley1 alcohol (m1) | 10 | 20 | — |
| Caprylic Acid (ml) | 5.0 | 5.0 | — |
| Tocopherol Aetate (mg) | q.s. | q.s. | q.s. |
| Mono/diglycerides of caprylic | — | — | Up to 100 |
| acid (ml) | | | |
| Light liquid paraffin (ml, upto) | 100 | 100 | — |

*q.s. Quantity sufficient

### Example 8

A) Preparation of Various Gel Compositions Devoid of C-15 or Greater Fatty Acids/Esters Containing Different Antifungal Agents.

Initially, carbopol was added to the water and allowed to swell for 24 hours. Antidandruff agent was dissolved in minimum quantity of solvent and added to the carbopol base, followed by neutralization with a dilute aqueous solution of triethanolamine or sodium hydroxide to obtain pH 5.0-7.0. The gel compositions were coded as 1G, 2G, 3G, 4G, 5G and 6G as shown in ‘Table 25’.

TABLE 25
Gel compositions containing antifungal agents
devoid of C-15 or greater fatty acids/esters

| | Compositions (wt %) | | | | | |
|---|---|---|---|---|---|---|
| Ingredients | 1G | 2G | 3G | 4G | 5G | 6G |
| Piroctone Olamine | 0.05 | 0.05 | 0.05 | 0.05 | — | |
| Ketoconazole | — | — | — | — | — | 0.016 |
| Zinc Pyrithione | — | — | — | — | 0.016 | — |
| Ethanol | 10.0 | 4.0 | 4.0 | 4.0 | — | — |
| Propylene glycol | — | — | — | — | — | 10 |
| PEG-400 | — | — | — | — | 10.8 | |
| Propylene glycol | — | 4.0 | 4.0 | 4.0 | 4.0 | 4.0 |
| mono caprylate | | | | | | |
| Polymeric | — | 0.2 | 0.2 | 0.2 | 0.2 | 0.2 |
| surfactant | | | | | | |
| Carbopols | 0.3 | 0.3 | 0.4 | 0.5 | 0.5 | 0.5 |
| Triethanolamine/ | q.s | q.s | q.s | q.s | q.s | q.s |
| sodium hydroxide | | | | | | |
| (pH adjuster) | | | | | | |
| Water | q.s. | q.s. | q.s. | q.s. | q.s. | q.s. |
| | (Balance) | (Balance) | (Balance) | (Balance) | (Balance) | (Balance) |

*q.s. Quantity sufficient

B) Study of Zone of Inhibition (ZOI) of Gel Compositions Devoid of C-15 or Greater Fatty Acids/Esters Containing Antifungal Agent Piroctone Olamine Against M. furfur under in vitro Conditions.

To study the efficacy of gel compositions, ZOI was determination by agar well diffusion method. Observations were shown in ‘Table 26’.

Table 26
Zone of inhibition of gel compositions containing piroetone and caprylic
acid and/or its ester olamine against M. furfur (MTCC1374)

| | Zone of Inhibition (in cm) | | |
|---|---|---|---|
| Formulations | Setup1 | Setup2 | Setup3 |
| IG | 1.2 | 1.0 | 0.9 |
| 2G | 1.4 | 1.5 | 1.3 |
| 1G with 10% oleic acid | — | — | — |
| Gel base without anti-fungal agent | — | — | — |
| (negative control) | | | |

Results:

As shown in ‘Table 26’, gel composition (1G) containing piroctone olamine showed ZOI (zone of inhibition) in the range of 1.2-0.9 cm against M. furfur (MTCC 1374). Whereas, composition (2G) with similar amount of piroctone olamine along with 4% propylene glycol mono caprylate showed ZOI 1.5-1.3 cm against M. furfur. After incorporation of 10% oleic acid with base formulation 1G, zone of inhibition was not observed. These results showed that the presence of oleic acid which is free fatty acids above C-14 has an adverse effect on the activity of the antifungal agent.

### Example 9: Preparation of Various Cream Compositions Devoid of C-15 or Greater Fatty Acids/Esters Containing Different Antifungal Agents

Creams were prepared by fusion method, where all oil soluble ingredients were weighed and melt at a temperature of 60-80° C. Aqueous phase was maintained at the same temperature and oil phase was poured into aqueous phase with constant stirring, followed by slow cooling with moderate stirring. The cream compositions were coded as 1C, 2C, 3C, 4C as shown in ‘Table 27’.

TABLE 27
Preparation of cream compositions containing antifungal agents piroctone
olamine or ketoconazole and caprylic acid ester derivative

| | Compositions (% wt) | | | |
|---|---|---|---|---|
| Ingredient | 1 C | 2 C | 3 C | 4 C |
| Piroctone Olamine | 0.05 | 0.1 | — | — |
| Ketoconazole | — | — | 2.0 | 1.0 |
| Lauryl alcohol | 6 | 6 | 6 | 6 |
| Steryl alcohol | 7 | 7 | 7 | 7 |
| Triglyceride of caprylic acid | 1.0 | 1.0 | 1.0 | 1.0 |
| Cyclomethicone | 7 | 7 | 7 | 7 |
| PEG2 ether of stearic acid | 0.5 | 0.5 | 0.5 | 0.5 |
| PEG21 ether of stearic acid | 2.5 | 2.5 | 2.5 | 2.5 |
| Propylene glycol | 5.0 | 5.0 | 5.0 | 5.0 |
| Carbopol | 0.25 | 0.25 | 0.25 | 0.25 |
| pH adjuster (sodium | q.s. | q.s. | q.s. | q.s. |
| hydroxide or citric acid) | | | | |
| Water | q.s. | q.s. | q.s. | q.s. |
| | (Balance) | (Balance) | (Balance) | (Balance) |

*q.s. Quantity sufficient

### Example 10: Preparation of Clotrimazole (1%) Topical Cream Formulations Containing at Least One or Two Medium Chain Fatty Acids (Between C-1 to C-14) or Ester Derivatives Thereof (Table 28)

Phase A: Purified water, carbopol 980, sodium hyaluronate

Phase B: Clotrimazole, cetostearyl alcohol, propylene glycol monocaprylate, glyceryl monocaprylate, glyceryl mono-di caprylate, propylene glycol monolaurate, glycerol monolaurate, diisopropyladipate, propylene glycol, mineral oil, cetomacrogol 1000, PEG-12-dimethicone, steareth 2, steareth 21,

Phase C: Benzyl alcohol, butylated hydroxy toluene

Phase D: Triethanolamine

Method of Preparation (F1):
(1) The ingredient(s) of phase A, like carbopol 980 is added slowly into water while maintaining the stirring speed at about 600-700 RPM till the polymers are homogenously suspended into water to form homogenous phase A (aqueous phase).
(2) All the ingredients of phase B (mineral oil, cresmer 1000, PEG 400, cetostearyl alcohol, propylene glycol monocaprylate, steareth 2, steareth 21 etc.) are mixed and melted at 70° C.
(3) Clotrimazole is added into phase B (oil phase) which is further added into phase A at 70° C. by maintaining stirring speed at about 200-300 RPM until homogeneous phase is obtained. The stirring is continued till the temperature of the final mixture reaches at 30-35° C.
(4) Phase C ingredients (butylated hydroxytoluene and benzyl alcohol) are added to the above homogeneous mixture while maintaining the stirring at about 200-300 RPM. Finally, the reaction mixture is neutralized with triethanolamine to attain final pH at about 6.5 to 7.0.

The exemplary cream formulations F2-F8 with the respective compositions as mentioned in Table 28 are prepared using similar method as used for F1.

TABLE 28
Clotrimazole (1%) topical cream formulations with at least
one or two medium chain fatty acids and derivatives thereof

| | Composition (% w/w) | | | | | | | | |
|---|---|---|---|---|---|---|---|---|---|
| | General | | | | | | | | |
| Chemical Name | composition | F1 | F2 | F3 | F4 | F5 | F6 | F7 | F8 |
| Clotrimazole | 1 | 1 | 1 | 1 | 1 | 1 | 1 | 1 | 1 |
| Cetostearyl alcohol | 1-10 | 7 | 6 | 2 | 2 | 7 | 7 | 7 | 7 |
| Propylene glycol | 1-10 | 8 | 8 | — | — | 5 | — | — | — |
| monocaprylate | (C-1 to C-14 | | | | | | | | |
| Glyceryl | fatty acids) | — | — | — | 8 | — | 5 | — | — |
| monocaprylate | | | | | | | | | |
| Glyceryl mono- | | — | — | 8 | — | — | — | 8 | — |
| dicaprylate/caprate | | | | | | | | | |
| Propylene glycol | | — | — | — | — | — | — | — | — |
| monolaurate | | | | | | | | | |
| Glycerol monolaurate | | — | — | — | — | — | — | — | 5 |
| Di-isopropyladipate | 1-8 | — | 4 | — | — | — | — | — | — |
| Propylene glycol | 1-10 | — | — | 4 | — | — | — | — | — |
| Mineral oil | 1-10 | 5 | — | — | — | 5 | 5 | 5 | 5 |
| Cetomacrogol 1000 | 1-5 | 1 | — | — | 2 | 1 | 1 | 1 | 1 |
| (Cresmer 1000) | | | | | | | | | |
| PEG 400 | 1-6 | 3 | — | — | — | 3 | 3 | 3 | 3 |
| PEG12-dimethicone | 1-10 | — | — | — | 3 | — | — | — | — |
| Steareth 2 | 1-4 | 2.5 | 3 | 3 | — | 2.5 | 2.5 | 2.5 | 2.5 |
| Steareth 21 | 1-4 | 2.5 | 3 | 3 | — | 2.5 | 2.5 | 2.5 | 2.5 |
| Carbopol 980 | 0.05-1.0 | 0.15 | 0.13 | 0.13 | 0.5 | 0.15 | 0.15 | 0.15 | 0.1 |
| Hydroxyethyl cellulose | 0.05-1.0 | | | | | | | | 0.1 |
| Sodium hyaluronate | 0.1-1 | — | — | — | 0.5 | — | — | — | — |
| Butylated | 0.1-0.2 | 0.1 | 0.1 | 0.1 | 0.1 | 0.1 | 0.1 | 0.1 | 0.1 |
| hydroxytoluene | | | | | | | | | |
| Benzyl alcohol | 0.1-2 | 1 | 1 | 1 | 1 | 1 | 1 | 1 | 1 |
| 10% Triethanolamine | | q.s | q.s | q.s | q.s | q.s | q.s | q.s | q.s |
| Water | | q.s | q.s | q.s | q.s | q.s | q.s | q.s | q.s |

### Example 11: Preparation of Luliconazole (1%) Topical Cream Formulations Containing at Least One or Two Medium Chain Fatty Acids or Derivatives Thereof (Table 29)

Method of Preparation (F9):

(1) The ingredient(s) of phase A, like carbopol 980 is added slowly into water while maintaining the stirring speed at about 600-700 RPM till the polymers are homogenously suspended into water to form homogenous phase A (aqueous phase).

(2) All the ingredient of phase B (cetostearyl alcohol, di-isopropyl adipate, propylene glycol monocaprylate, steareth 2, steareth 21 etc.) are mixed and melted at 70° C.

(3) Luliconazole is added into Phase B (oil phase) which is further added into phase A at 70° C. by maintaining stirring speed at about 200-300 RPM until homogeneous phase is obtained. The stirring is continued till the temperature of the final mixture reaches at 30-35° C.

(4) Phase C ingredients (butylated hydroxytoluene and benzyl alcohol) are added to above homogeneous mixture while maintaining the stirring at about 200-300 RPM. Finally, the reaction mixture is neutralized with triethanolamine to attain final pH at about 6.5 to 7.0.

The exemplary cream formulations F10-F14 with the respective compositions as mentioned in Table 29 are prepared using similar method as used for F9.

TABLE 29
Luliconazole (1%) topical cream formulation with at least
one or two medium chain fatty acids and derivatives thereof

| | General | Composition (% w/w) | | | | | |
|---|---|---|---|---|---|---|---|
| Chemical name | composition | F9 | F10 | F11 | F12 | F13 | F14 |
| Luliconazole | 1 | 1 | 1 | 1 | 1 | 1 | 1 |
| Cetostearyl alcohol | 1-10 | 6 | 5 | 3 | 4 | 6 | 6 |
| Propylene glycol | 1-10 | 8 | — | — | 8 | 5 | — |
| monocaprylate | (C-1 to C-14 | | | | | | |
| Glyceryl | fatty acids) | — | 8 | — | — | — | 3 |
| monocaprylate | | | | | | | |
| Glyceryl mono- | | — | — | 8 | — | — | — |
| dicaprylate/caprate | | | | | | | |
| Propylene glycol | | | | | | | 2 |
| monolaurate | | | | | | | |
| Di-isopropyl adipate | 1-6 | 4 | — | 4 | 4 | 4 | 4 |
| Proplylene glycol | 1-10 | — | 2 | — | 4 | — | — |
| Steareth 2 | 1-4 | 3 | 3 | 3 | 3 | 3 | 3 |
| Steareth 21 | 1-4 | 3 | 3 | 3 | 3 | 3 | 3 |
| Carbopol 980 | 0.05-1 | 0.1 | 0.1 | 0.2 | 0.1 | 0.1 | 0.1 |
| Hydroxyethyl cellulose | 0.05-1 | | | | | | |
| Sodium hyaluronate | 0.05-0.5 | — | 0.1 | 0.1 | 0.2 | — | — |
| Butylated | 0.1-0.2 | 0.1 | 0.1 | 0.1 | 0.1 | 0.1 | 0.1 |
| hydroxytoluene (BHT) | | | | | | | |
| Benzyl alcohol | 0.1-2 | 1 | 1 | 1 | 1 | 1 | 1 |
| 10% Triethanolamine | q.s | q.s | q.s | q.s | q.s | q.s | q.s |
| Water | q.s | q.s | q.s | q.s | q.s | q.s | q.s |

### Example 12: Preparation of terbinafine (1%) topical cream formulation with at least one or two medium chain fatty acid and fatty acid derivatives (Table 30)

Phase A: Water, propylene glycol, tween 20

Phase B: Terbinafine HCl, cetyl alcohol, stearyl alcohol, cetostearyl alcohol, propylene glycol monocaprylate, dimethyl isosorbide, steareth 2, steareth 21, dimethicone

Phase C: Carbopol 980, hydroxypropylcellulose

Phase D: Benzyl alcohol, BHT, glycerine

Phase E: Triethanolamine

Method of preparation (F15):

(1) The ingredient(s) of phase A containing water and propylene glycol

(2) All the ingredient of phase B (cetyl alcohol, stearyl alcohol, propylene glycol monocaprylate, dimethyl isosorbide, steareth 2, steareth 21) are mixed and melted at 70° C.

(3) Terbinafine HCL is added into Phase B (oil phase) which is further added into phase A at 70° C. by maintaining stirring speed at about 200-300 RPM until homogeneous phase is obtained. The stirring is continued till the temperature of the final mixture reaches at 30-35° C.

(4) Phase C ingredients (benzyl alcohol) are added to above homogeneous mixture while maintaining the stirring at about 200-300 RPM. Finally, the reaction mixture is neutralized with triethanolamine to attain final pH at about 6.5 to 7.0.

The exemplary cream formulations F16-F20 with the respective compositions as mentioned in Table 30 are prepared using similar method as used for F15.

TABLE 30
Terbinafine (1%) topical cream formulation with at least one
or two medium chain fatty acids and derivatives thereof

| | General | Composition (% w/w) | | | | | |
|---|---|---|---|---|---|---|---|
| Chemical Name | composition | F15 | F16 | F17 | F18 | F19 | F20 |
| Terbinafine HCL | 1 | 1 | 1 | 1 | 1 | 1 | 1 |
| Cetostearyl alcohol | 1-8 | | | | | 6 | 2 |
| Cetyl alcohol | 1-10 | 2 | 2.7 | 2.7 | 2.7 | | |
| Stearyl alcohol | 1-10 | | 2.7 | 2.7 | 2.7 | | |
| Propylene glycol | 1-10 | 1 | 3 | 5 | 8 | 5 | 4 |
| monocaprylate | | | | | | | |
| Glycerol caprylate/ | 1-10 | | | | | | |
| caprate | | | | | | | |
| Propylene glycol | | | | | | | 4 |
| monolaurate | | | | | | | |
| Dimethyl isosorbide | 1-10 | 5 | 5 | 5 | 5 | 5 | |
| Steareth 2 | 1-4 | 2 | 3 | 3 | 3 | 3 | |
| Steareth 21 | 1-4 | 3 | 3 | 3 | 3 | 3 | |
| Tween 20/Tween 80 | 1-4 | | | | | | 2 |
| Dimethicone | 1-4 | | | | | | 2 |
| Glycerine | 1-4 | | | | | | 3 |
| Propylene Glycol | 1-10 | 5 | 5 | 5 | 5 | 4 | |
| Carbopol 980 | 0.05-1 | | | | | | 7.5 |
| Hydroxyethyl cellulose | 0.05-1 | | | | | | 7.5 |
| Benzyl alcohol | 0.1-2 | 1 | 1 | 1 | 1 | 1 | 1 |
| Butylated hydroxy toluene | 0.1-0.2 | | | | | | 0.1 |
| (BHT) | | | | | | | |
| 10% Triethanolamine | q.s | q.s | q.s | q.s | q.s | q.s | q.s |
| Water | q.s | q.s | q.s | q.s | q.s | q.s | q.s |

### Example 13: Preparation of Luliconazole (1%) Topical Lotion Formulation without Ethanol and Containing at Least One or Two Medium Chain Fatty Acid and Fatty Acid Derivatives (Table 31)

Phase A: Water, propylene glycol, PEG 300, PEG 400, ethanol, isopropyl alcohol.

Phase B: Luliconazole, propylene glycol monocaprylate, diethylene glycol, diisopropyladipate, ceteareth 20, PEG-12-dimethicone, oleth 20

Phase C: Benzyl alcohol, butylated hydroxy toluene

Method of Preparation (LN/01):

(1) In main mixing vessel water, propylene glycol and PEG 400 are added and heated up to 40-50° C. (Phase A)

(2) In a separate vessel, propylene glycol monocaprylate, diethylene glycol monoethyl ether, PEG-12 dimethicone, ceteareth 20 are added and heated at 40-50° C. to form a transparent solution. Luliconazole is solubilized in the final mixture while stirring at 100 rpm (Phase B).

(3) Phase B contents is added into phase A slowly with stirring. The stirring is continued till the temperature of the final mixture reaches at 30-35° C.

(4) BHT is solubilized in benzyl alcohol, and add into the final mixture vessel at 30-35° C.

(5) Clear transparent lotion is obtained.

The exemplary lotion formulations LN02-LN08 with the respective compositions as mentioned in Table 31 are prepared using similar method as used for LN01.

TABLE 31
Luliconazole (1%) topical lotion formulation without ethanol containing
at least one or two medium chain fatty acid and derivatives thereof

| | Composition (% w/w) | | | | | | | | |
|---|---|---|---|---|---|---|---|---|---|
| | General | LN/ | LN/ | LN/ | LN/ | LN/ | LN/ | LN/ | LN/ |
| Chemical Name | Composition | 01 | 02 | 03 | 04 | 05 | 06 | 07 | 08 |
| Luliconazole | 1 | 1 | 1 | 1 | 1 | 1 | 1 | 1 | 1 |
| Propylene glycol | 3-12 | 8 | 5 | 5 | 5 | 5 | 5 | 8 | |
| mono caprylate | | | | | | | | | |
| Propylene glycol | | | | | | | | | 8 |
| mono laurate | | | | | | | | | |
| Diethyiene glycol | 1-40 | 10 | 10 | 10 | 10 | 5 | 35 | | 10 |
| monoethylether | | | | | | | | | |
| Diisopropyladipate | 1-5 | | | | | | | 5 | |
| Ceteareth 20 | 3-8 | 6 | 6 | 6 | 6 | 1 | 1 | | 6 |
| PEG-12- | 3-8 | 4 | 4 | 4 | 4 | | | | 4 |
| dimethicone | | | | | | | | | |
| Oleth 20 | 5-10 | | | | | 7 | 7 | | |
| Propylene Glycol | 10-50 | 15 | 15 | | | | | 45 | 15 |
| 1,3 Propanediol | 10-50 | | | 30 | 30 | 30 | | | |
| PEG 300 | 5-25 | | | | | | | 20 | |
| PEG 400 | 5-25 | 20 | 20 | 5 | | | | 20 | 20 |
| Isopropyl alcohol | 1-20 | | | | 5 | 5 | | | |
| Benzyl alcohol | 0.1-2 | 1 | 1 | 1 | 1 | 1 | 1 | 1 | 1 |
| Butylated hydroxy | 0.01-0.2 | 0.1 | 0.1 | 0.1 | 0.1 | 0.1 | 0.1 | 0.1 | 0.1 |
| toluene (BHT) | | | | | | | | | |
| Water | q.s. | q.s | q.s. | q.s | q.s. | q.s | q.s. | | q.s |

### Example 14: Preparation of Luliconazole (1%) Topical Lotion Formulation with Ethanol Containing at Least One or Two Medium Chain Fatty Acid and Fatty Acid Derivatives (Table 32)

Phase A: Water, propylene glycol, PEG 300, PEG 400, ethanol, isopropyl alcohol, 1,3 propanediol

Phase B: Luliconazole, propylene glycol monocaprylate, diethylene glycol, diisopropyladipate, ceteareth 20, PEG-12-dimethicone, oleth 20

Phase C: Benzyl alcohol, butylated hydroxy toluene.

Method of Preparation (LN/09):

(1) In main mixing vessel water, propylene glycol and PEG 400, ethanol is added and heated up to 40-50° C. (Phase A).

(2) In a separate vessel, propylene glycol monocaprylate, diethylene glycol monoethyl ether, PEG-12 dimethicone, ceteareth 20 are added and heated at 40-50° C. to form a transparent solution. Luliconazole is solubilized in the final mixture while stirring at 100 rpm (Phase B).

(3) Phase B contents is added into phase A slowly with stirring. The stirring is continued till the temperature of the final mixture reaches at 30-35° C.

(4) BHT is solubilized in benzyl alcohol, and add into the final mixture vessel at 30-35° C.

(5) Clear transparent lotion is obtained.

The exemplary lotion formulations LN10-LN15 with the respective compositions as mentioned in Table 32 are prepared using similar method as used for LN09.

TABLE 32
Luliconazole (1%) topical lotion formulation with ethanol containing
at least one or two medium chain fatty acid and derivatives thereof

| | Composition (% w/w) | | | | | | | |
|---|---|---|---|---|---|---|---|---|
| | General | LN/ | LN/ | LN/ | LN/ | LN/ | LN/ | LN/ |
| Chemical Name | Composition | 09 | 10 | 11 | 12 | 13 | 14 | 15 |
| Luliconazole | 1 | 1 | 1 | 1 | 1 | 1 | 1 | 1 |
| Propylene glycol | 3-12 | 8 | 5 | 5 | 5 | 5 | 5 | 8 |
| mono caprylate | | | | | | | | |
| Diethylene glycol | 1-40 | 10 | 10 | 10 | 10 | 5 | 35 | |
| monoethylether | | | | | | | | |
| Di-isopropyladiapte | 1-20 | | | | | | | 5 |
| Ceteareth 20 | 3-8 | 6 | 6 | 6 | 6 | 1 | 1 | |
| PEG-12- | 3-8 | 4 | 4 | 4 | 4 | | | |
| dimethicone | | | | | | | | |
| Oleth 20 | 5-10 | | | | | 7 | 7 | |
| Propylene glycol | 10-50 | 15 | 15 | | | | | 45 |
| 1,3 Propanediol | 10-50 | | | 30 | 30 | 30 | | |
| PEG 300 | 5-25 | | | | | | | 20 |
| PEG 400 | 5-25 | 20 | 20 | 5 | | | | 20 |
| Ethanol | 1-20 | 5 | 10 | 5 | 5 | 5 | 5 | 5 |
| Benzyl alcohol | 0.1-2 | 1 | 1 | 1 | 1 | 1 | 1 | 1 |
| Butylated hydroxy | 0.01-0.2 | 0.1 | 0.1 | 0.1 | 0.1 | 0.1 | 0.1 | 0.1 |
| toluene | | | | | | | | |
| Water | q.s. | q.s. | q.s. | q.s | q.s. | q.s | q.s. | q.s |

### Example 15: Preparation of Efinaconazole (1%) Topical Nail Solution with at Least One or Two Medium Chain Fatty Acid and Fatty Acid Derivatives (Table 33)

Method of Preparation (NL/01):

(1) The film forming resin Acrycoat E-100 or Eudragit RL100 is dissolved in ethanol (Phase B).

(2) Propylene glycol monocaprylate and efinaconazole are dissolved in mixture of solvents like diisopropyl adipate, ethanol, butyl acetate and ethyl acetate at room temperature while stirring at 50-100 rpm to prevent evaporation of volatile solvent (Phase A).

(3) Phase B is slowly added into phase A to form homogenous transparent solution while stirring the final mixture at 50-100 rpm.

(4) Finally, the pH of the solution was adjusted to pH 4-6 by using suitable pH modifier or buffering agent is added to prevent pH change in the final formulation.

The exemplary nail solutions NL02-NL03 with the respective compositions as mentioned in Table 33 are prepared using similar method as used for NL01.

TABLE 33
Efinaconazole (1%) topical nail solutions with at least one or two
medium chain fatty acid and derivatives thereof

| | Composition (% w/w) | | | |
|---|---|---|---|---|
| | General | | | |
| Chemical Name | Composition | NL/01 | NL/02 | NL/03 |
| Efinaconazole | 1-10 | 5 | 5 | 10 |
| Propylene Glycol mono caprylate | 1-10 | 2.1 | 2 | 2 |
| Diisopropyl adipate | 1-6 | 4 | 4 | 4 |
| Ethanol | 10-70 | 57 | 67 | 62 |
| Butyt acetate | 10-70 | 18 | 21 | 14 |
| Ethyl acetate | 10-70 | 22 | 10 | 16 |
| Acrycoat E-100/Eudragit RL100 | 0.5-5 | 2 | 1 | |
| Plasticizer ATBC | 0.5-5 | 1.9 | 2 | 2 |
| Acrylic resin | 0.5-5 | | | 2 |
| pH modifier | q.s. | q.s. | q.s. | q.s. |

### Example 16: Ketoconazole and Ketoconazole+Zinc Pyrithione Combination Shampoo Composition with Structured Surfactants Containing at Least One or Two Medium Chain Fatty Acid and Fatty Acid Derivatives (Table 34)

Phase A: Water, Guar hydroxypropyltrimonium chloride, citric acid solution

Phase B: Trisodium ethylenediamine disuccinate

Phase C: sodium chloride solution

Phase D: Iselux SLC. Miracare SLB 365, sodium lauryl ether sulphate, sodium lauryl suphate, cocaamidopropylbetaine, sodium cocoyl methyl taurate solution

Phase E: Ketoconazole, glyceryl mono/di-caprate and caprylate, propylene glycol monocaprylate, laureth 4, laureth 23

Phase F: Zinc pyrithione solid or dispersion

Phase G: Phenoxyethanol, Fragrance

Phase H: Citric acid solution

Phase I: Colorant

Method of Preparation (SH/01):
(1) In a main mixing vessel, Guar hydroxypropyltrimonium chloride is dispersed in water, and traces of citric acid solution is added to attain pH 5-5.5. The transparent solution was allowed to swell for 10 minutes while stirring at 200 rpm.
(2) Trisodium ethylenediamine disuccinate and sodium chloride solution are added into the above mixture while stirring at 200 rpm.
(3) Specified amount of Iselux SLC is added into the main mixing vessel at 50 rpm and allowed to mix for 10 minutes followed by addition of sodium cocoyl methyl taurate solution. The final mixture is stirred at 80-100 rpm for 10-15 minutes until uniform solution is obtained. The solution is heated at 50° C.
(4) In another vessel ketoconazole is dissolved in mixture of propylene glycol mono caprylate and glyceryl mono/di-caprate/caprylate while heating the mixture at 50-60° C. till ketoconazole dissolves.
(5) Ketoconazole solubilized solution was added into the main mixing vessel at 50° C., and stirred at 50-100 rpm for 5 minutes until homogeneous phase is obtained. The stirring is continued till the temperature of the final mixture reaches at 30-35° C.
(6) Phase G is added into the main mixing vessel.
(7) Citric acid solution is added to the final mixture to adjust the pH 6-7.
(8) Colorant (if any) is going to be added at last to obtain final shampoo formulation.

The exemplary shampoo formulations SH02-SH08 with the respective compositions as mentioned in Table 34 are prepared using similar method as used for SH01.

TABLE 34
Ketoconazole or ketoconazole + zinc pyrithione combination
shampoo composition with structured surfactants containing at
least one or two medium chain fatty acid and derivatives thereof

| | Composition (% wt/wt) | | | | | | | | |
|---|---|---|---|---|---|---|---|---|---|
| | General | SH/ | SH/ | SH/ | SH/ | SH/ | SH/ | SH/ | SH/ |
| Chemical name | Composition | 01 | 02 | 03 | 04 | 05 | 06 | 07 | 08 |
| Ketoconazole | 0.5-4 | 2 | 2 | 2 | 2 | 2 | 2 | 2 | 2 |
| Zinc pyrithione | 0.1-2 | | | | | | | | 1 |
| (ZPTO) | | | | | | | | | |
| Propylene glycol | 1-10 | 5 | 5 | 4 | 5 | | 3 | 3 | 3 |
| monocaprylate | | | | | | | | | |
| Glyceryl mono- | 1-12 | 3 | | 4 | | 8 | 5 | 5 | 5 |
| dicaprate/caprylate | | | | | | | | | |
| Guar | 0.1-5 | 0.2 | 0.2 | 0.2 | 0.2 | 0.2 | 0.2 | 0.2 | 0.2 |
| Hydroxypropyl | | | | | | | | | |
| trimonium chloride | | | | | | | | | |
| Trisodium | 0.1-1 | 0.5 | 0.5 | 0.5 | 0.5 | 0.5 | 0.5 | 0.5 | 0.5 |
| ethylenediamine | | | | | | | | | |
| disuccinate | | | | | | | | | |
| Sodium chloride | 0.1-5 | 2 | 2 | 2 | 2 | 2 | 2 | 3 | 3 |
| Sodium cocoyl | 2-30 | 12 | | 7 | 10 | 10 | 15 | | |
| methyl taurate | | | | | | | | | |
| (32% paste form) | | | | | | | | | |
| Laureth 4 | 0.1-2 | 0.5 | | | | | | | 0.5 |
| Laureth 23 | 0.1-5 | 1 | | | | | | | 1 |
| Sodium Lauryl | 5-40 | | | | | | | | 25 |
| ether sulphate | | | | | | | | | |
| Sodium lauryl | 5-40 | | | | | | | | 15 |
| sulphate | | | | | | | | | |
| Cocaamidopropyl | 1-20 | | | | | | | | 5 |
| betaine | | | | | | | | | |
| Iselux SLC (Water, | 10-40 | 18 | 30 | 18 | 15 | 20 | 18 | | |
| sodium lauroyl | | | | | | | | | |
| methyl isethionate, | | | | | | | | | |
| sodium | | | | | | | | | |
| lauroamphoacetate | | | | | | | | | |
| and cocamide MIPA) | | | | | | | | | |
| Miracare SLB 365 | | | | | | | | 35 | |
| (water + sodium | | | | | | | | | |
| trideceth sulfate, | | | | | | | | | |
| sodium | | | | | | | | | |
| lauroamphoacetate, | | | | | | | | | |
| cocamide MEA, | | | | | | | | | |
| sodium chloride, | | | | | | | | | |
| methyl- | | | | | | | | | |
| isothiazolinone) | | | | | | | | | |
| Phenoxyethanol | 0.5-2 | 0.6 | 0.6 | 0.6 | 0.6 | 0.6 | 0.6 | 0.6 | 0.6 |
| Butylated | 0.05-0.1 | | | | 0.1 | 0.1 | 0.1 | — | — |
| hydroxytoluene | | | | | | | | | |
| Fragrance | q.s | q.s | q.s | q.s | q.s | q.s | q.s | q.s | q.s |
| Citric acid solution | q.s. | q.s. | q.s. | q.s. | q.s. | q.s. | q.s. | q.s. | q.s. |
| (pH modifier) | | | | | | | | | |
| Water | q.s. | q.s. | q.s. | q.s. | q.s. | q.s. | q.s. | q.s. | q.s. |
| Colorant | q.s. | q.s. | q.s. | q.s. | q.s. | q.s. | q.s. | q.s. | q.s. |

### Example 17: Preparation of Ketoconazole Shampoo Composition with Mild Sulfate Free Surfactants Containing at Least One or Two Medium Chain Fatty Acid and Fatty Acid Derivatives (Table 35)

Phase A: Purified water, carbopol aqua SF-2, guar hydroxypropyltrimonium chloride

Phase B: Water, sodium lauroyl sarcosinate, sodium lauroamphoacetate, cocamide MEA water, cocaamidopropyl betaine

Phase C: Ketoconazole, glyceryl caprate/caprylate, propylene glycol monocaprylate

Phase F: Phenoxyethanol, butylated hydroxy toluene.

Phase G: Fragrance

Phase H: Citric acid solution

Phase I: Colorant

Method of Preparation (SH/09)
(1) In a main mixing vessel, carbopol aqua SF-2 is dispersed in water.
(2) In a separate vessel all the ingredients of phase B (sodium lauroyl sarcosinate, sodiumlauroamphoacetate, cocamide MEA, cocaamidopropyl betaine) are taken and heated at 50-60° C. at 50 rpm till all the contents get dissolved.
(3) Phase B is added into the main mixing vessel maintaining the temperature at 50-60° C. while stirring the mixture at 100 rpm.
(4) In a separate vessel ketoconazole is dissolved in mixture of propylene glycol mono caprylate and glyceryl mono/di-caprate/caprylate while heating the mixture at 50-60° C. till ketoconazole dissolves.
(5) Ketoconazole solubilized solution was added into the main mixing vessel at 50° C., and stirred at 50-100 rpm for 5 minutes until homogeneous phase is obtained. The stirring is continued till the temperature of the final mixture reaches at 30-35° C.
(6) Phase F and G are added sequentially while stirring the final solution for half an hour till the homogenous solution is obtained.
(7) Citric acid solution is added to adjust the pH 6-7.
(8) Colorant (if any) is going to be added at last to obtain final shampoo formulation.

The exemplary shampoo formulations SH10-SH11 with the respective compositions as mentioned in Table 35 are prepared using similar method as used for SH09.

TABLE 35
Ketoconazole shampoo composition with mild sulfate free surfactants
containing at least one or two medium chain fatty acid and derivatives
thereof

| | Composition (% wt/wt) | | | |
|---|---|---|---|---|
| | General | | | |
| Chemical name | composition | SH/09 | SH/10 | SH/11 |
| Ketoconazole | 0.5-3 | 2 | 7 | 2 |
| Propylene glycol monocaprylate | 1-10 | 8 | 8 | 4 |
| Glyceryl caprate/caprylate | 1-12 | | | 4 |
| Carbopol aqua SF-2 | 1-10 | 2 | 2 | |
| Guar hydroxypropyl triammonium | 0.1-2 | | | 0.2 |
| chloride | | | | |
| Sodium lauroyl sarcosinate | 5-25 | 7 | 7 | 10 |
| Sodium lauroamphoacetate | 5-15 | 7 | 7 | 8 |
| Cocamide MEA | 0.5-2 | 2 | 2 | 2 |
| (Cocomonoethanolamide) | | | | |
| Disodium cocoamphodiacetate | 2-20 | | | 8 |
| Cocamidopropyl betaine | 1-20 | 7 | 7 | |
| Phenoxyethanol | 0.5-2 | 0.6 | 0.6 | 0.6 |
| Fragrance | q.s. | q.s. | q.s. | q.s. |
| Citric acid solution to adjust pH) | q.s. | q.s. | q.s. | q.s. |
| Butylated hydroxytoluene | 0.05-0.1 | 0.1 | 0.1 | 0.1 |
| Purified Water | q.s. | q.s. | q.s. | q.s. |
| Colorant | q.s. | q.s. | q.s. | q.s. |

### Example 18: In Vitro Fungal Kill Efficacy of a Terbinafine Formulation Containing C-1 to C-14 Fatty Acid or It's Ester (Propylene Glycol Monocaprylate) Against Terbinafine Resistant Trichophyton interdigitale (GTB-2S) in a Time Kill Assay

Method:

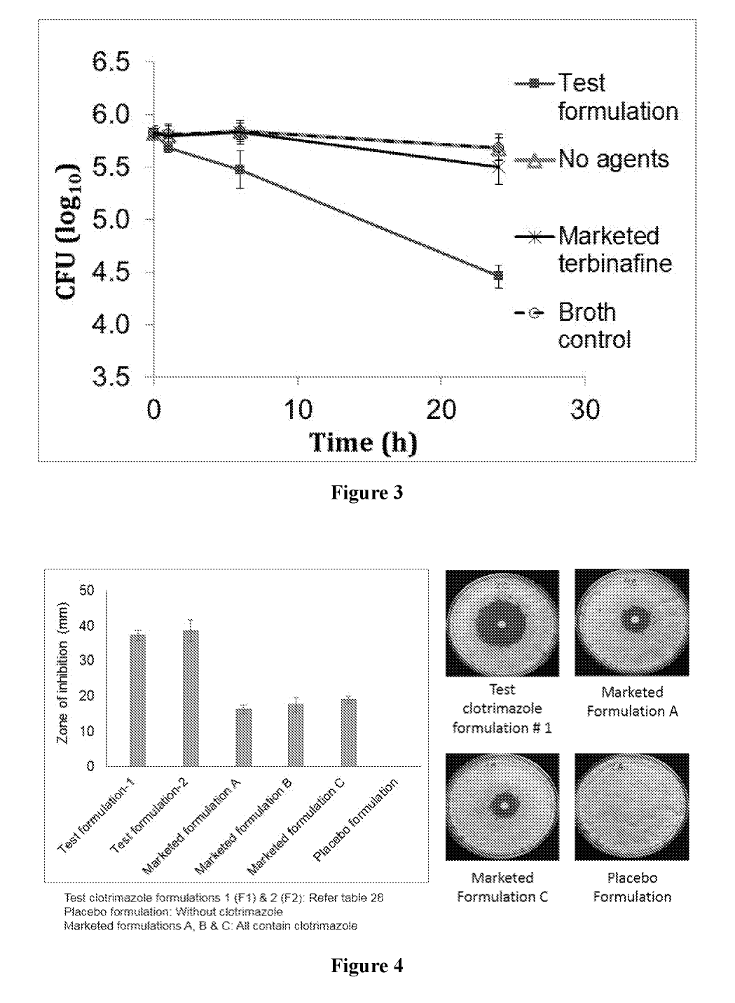

In vitro time kill assays were performed against Trichophyton interdigitale (GTB-2S) using 100 times diluted terbinafine cream formulations in Sabouraud dextrose broth (SDB). The Trichophyton inoculum was adjusted to 1 McFarland and exposed to terbinafine formulations (100 times diluted) for various durations (1, 6 and 24 h). At the end of each incubation period, cells were serially diluted and plated on Sabouraud dextrose agar (SDA) plates. Plates were incubated at 37° C. for 5 days following which the total colony forming units (CFU) were counted. Experiment was performed in triplicates and data was plotted as changes in number of CFU over time (FIG. 3).

Results:

The terbinafine formulation (test formulation) containing propylene glycol monocaprylate was effective in reducing the terbinafine resistant Trichophyton load whereas terbinafine alone formulation (marketed terbinafine, Ranbaxy Laboratories Ltd) was found to be ineffective (FIG. 3).

### Example 19: In Vitro Potency of a Clotrimazole Formulation Containing C-1 to C-14 Fatty Acid or It's Ester (Dropylene Glycol Monocaprylate) Against an Azole Resistant T. rubrum Strain (GTB-3FR-TS) in Zone of Inhibition Assays

Method:

In vitro fungal killing efficacy of various clotrimazole formulations were studied using ZOI assays. Each clotrimazole formulation (1%) was diluted (1:10 in sterile water). Sterile discs for ZOI assays were placed in the center of plates inoculated with an azole resistant T. rubrum. 10 μl of the diluted formulations were loaded onto each disc and the plates were incubated for 5 days at 37° C. The zones of inhibition were measured at the end of the incubation period. The assay was performed in triplicates for each formulation.

Results:

Clotrimazole formulations (test formulations) containing propylene glycol monocaprylate outperformed marketed clotrimazole formulations (clotrimazole creams from Glenmark Pharmaceuticals Ltd and Bayer Pharmaceuticals Pvt. Ltd.) and showed distinctively larger zone of inhibition against the azole resistant T. rubrum (FIG. 4).

### Example 20: In Vitro Potency of a Clotrimazole Formulation Containing C-1 to C-14 Fatty Acid or It's Ester (propylene Glycol monocaprylate) Against an Azole Resistant C. albicans Strain (MTCC 227) in Zone of Inhibition Assays (ZOI)

Method:

In vitro fungal killing efficacy of various clotrimazole formulations against azole resistant C. albicans (MTCC 227) were performed using ZOI assays. Each clotrimazole formulation (1%) was diluted (1:10 in sterile water). Sterile discs for ZOI assays were placed in the center of plates inoculated with C. albicans. 10 μl of the diluted formulations were loaded onto each disc and the plates were incubated for 24 h at 32° C. The zones of inhibition were measured at the end of the incubation period. The assay was performed in triplicates for each formulation.

Results:

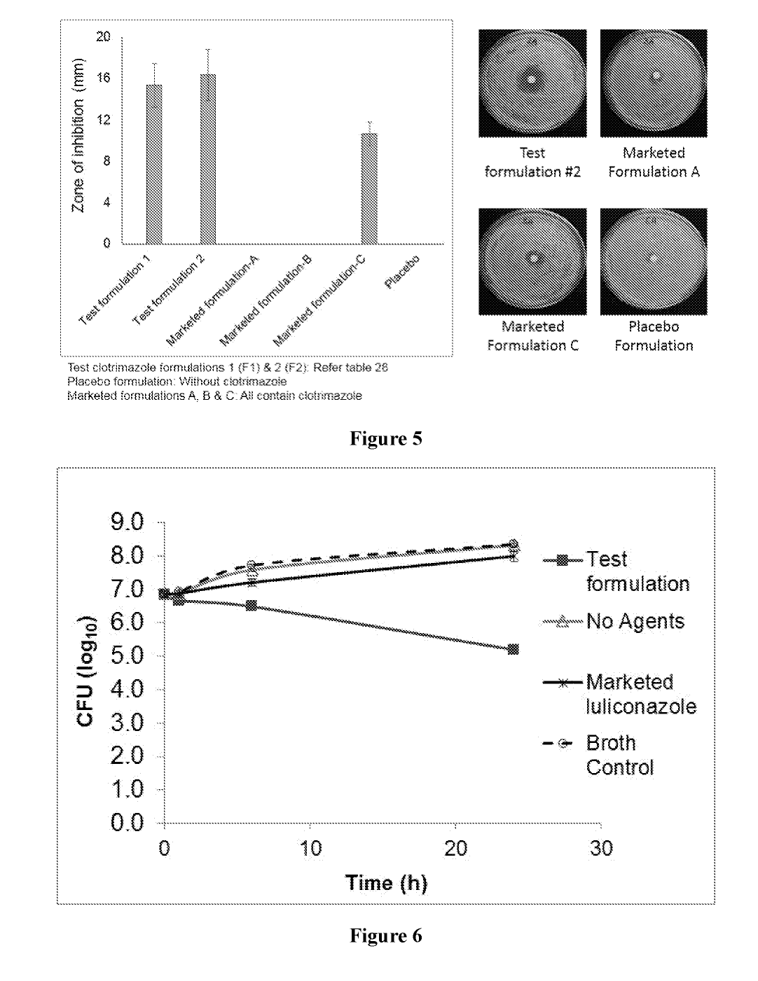

Clotrimazole formulations (test formulations) containing propylene glycol monocaprylate outperformed marketed clotrimazole formulations (clotrimazole creams from Glenmark Pharmaceuticals Ltd and Bayer Pharmaceuticals Pvt. Ltd.) and showed distinctively larger zone of inhibition against the azole resistant C. albicans (FIG. 5).

### Example 21: In Vitro Fungal Kill Efficacy of a Luliconazole Formulation Containing C-1 to C-14 Fatty Acid or It's Ester (Propylene Glycol Monocaprylate) Against Azole Resistant C. albicans (MTCC 227) in a Time Kill Assay

Method:

In vitro time kill assays were performed against C. albicans (MTCC 227) using 100 times diluted luliconazole cream formulations in Sabouraud dextrose broth (SDB). The Candida inoculum was adjusted to 1 McFarland and exposed to luliconazole formulations (100 times diluted) for various durations (1, 6 and 24 h). At the end of each incubation period, cells were serially diluted and plated on Sabouraud dextrose agar (SDA) plates. Plates were incubated at 32° C. for 24 h following which the total colony forming units (CFU) were counted. Experiment was performed in triplicates and data was plotted as changes in number of CFU over time (FIG. 6).

Results:

The luliconazole formulation containing propylene glycol monocaprylate (test formulation) was effective in reducing the azole resistant Candida load whereas luliconazole marketed formulation (Sun Pharmaceutical Ind. Ltd.) was found to be ineffective (FIG. 6).

### Example 22: In Vitro Potency of a Luliconazole Formulation Containing C-1 to C-14 Fatty Acid or It's Ester (Propylene Glycol Monocaprylate) Against an Azole Resistant C. albicans Strain (MTCC 227) in Zone of Inhibition Assays

Method:

In vitro fungal killing efficacy comparison of various luliconazole formulations were performed using ZOI assays. Each luliconazole formulation (1%) was diluted (1:10 in sterile water). Sterile discs for ZOI assays were placed in the center of plates inoculated with an azole resistant C. albicans. 10 μl of the diluted formulations were loaded onto each disc and the plates were incubated for 24 h at 32° C. The zones of inhibition were measured at the end of the incubation period. The assay was performed in triplicates for each formulation.

Results:

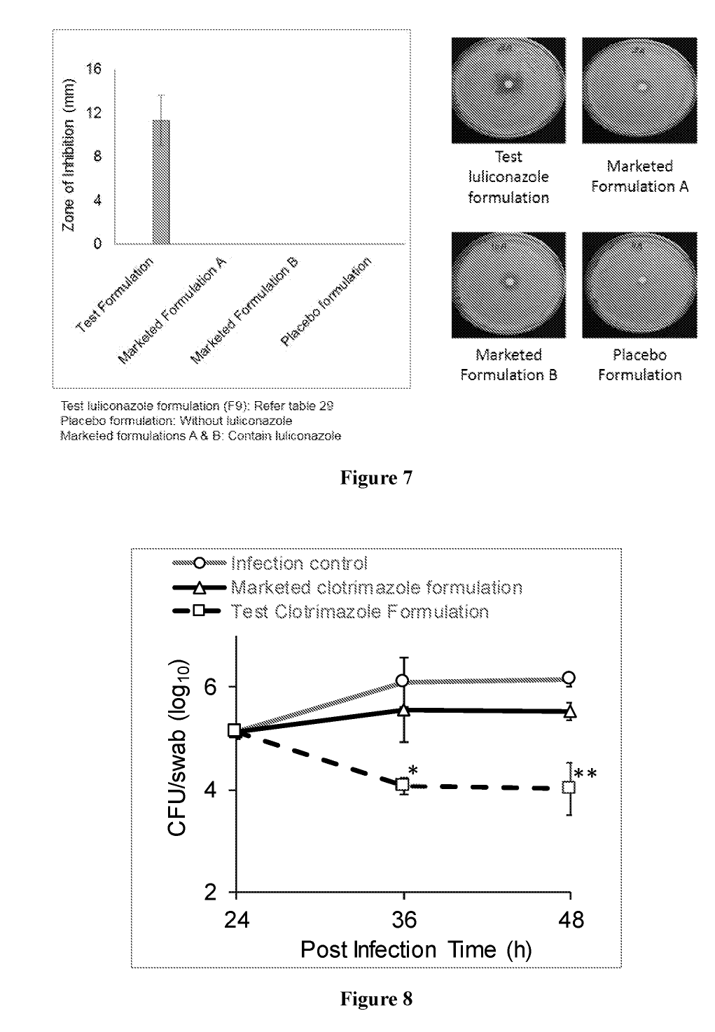

Luliconazole formulations containing propylene glycol monocaprylate (test formulation) outperformed marketed luliconazole formulations (luliconazole 1% creams from Glenmark pharmaceuticals Ltd and Sun Pharmaceutical Ind. Ltd.) and showed distinctively larger zone of inhibition against the azole resistant C. albicans (FIG. 7).

### Example 23: In Vivo Efficacy of Clotrimazole Formulation Containing C-1 to C-14 Fatty Acid or It's Ester (Propylene Glycol Monocaprylate) in a Model of Cutaneous Candidiasis

Method:

The efficacy of formulation of clotrimazole and propylene glycol monocaprylate was evaluated in a neutropenic murine skin infection model with azole resistant Candida albicans. The dorsal skin of mice was shaved, lightly scarified/abraded (1×1 cm) within a standard measured by a grid with a scalpel, following which 40 μl suspension of C. albicans (MTCC 227) (1×10⁹ CFU), was applied to the abraded skin. Treatment with antifungal agents were started after 24 h post-infection once daily at a dose of 15 mg/animal till 48 h. Fungal counts were measured 12 and 24 h post treatment by swabbing the infected area and plating the collected sample.

Results:

The fungal load reduction mediated by clotrimazole formulation containing propylene glycol monocaprylate (test clotrimazole formulation) was clearly superior to the marketed clotrimazole formulation (Glenmark Pharmaceuticals Ltd) that failed to cause any reduction in the infection load in this model of cutaneous candidiasis in mice (FIG. 8).

### Example 24: In Vivo Efficacy of Luliconazole Formulation Containing Containing Propylene Glycol Monocaprylate in an Animal Model of Tinca Infection

Method:

The efficacy of formulation of luliconazole and propylene glycol monocaprylate was evaluated in a neutropenic murine skin infection model with a pathogenic T. mentagrophyte strain (ATCC 24953). The dorsal skin of mice was shaved, lightly scarified/abraded (1×1 cm) within a standard measured by a grid with a scalpel, following which 0.05 ml suspension of T. mentagrophytes culture (ATCC 24953) (5×10⁶ CFU/animal), was applied to the abraded skin. Treatment with antifungal agents were started at day 5, post-infection twice daily at a dose of 15 mg/animal for 10 days till day 14. Lesion score was measured at every 5 days from the infection date till day 14 and continued to day 21 to check for recurrence if any. Skin lesions were from 0 to 4 based on the severity of the lesions.

Results:

The lesion score was significantly reduced with luliconazole formulation containing propylene glycol monocaprylate (test luliconazole formulation) compared to infected control at day 15 and day 21. The formulation was found to be superior compared to marketed luliconazole formulation (FIG. 9).

### Example 25: In Vitro Fungal Kill Efficacy of Ketoconazole Shampoo Formulations Containing c Containing C-1 to C-14 Fatty Acid or It's Ester (Caprylic Acid Esters) Against M. furfur in a Time Kill Assay

Method:

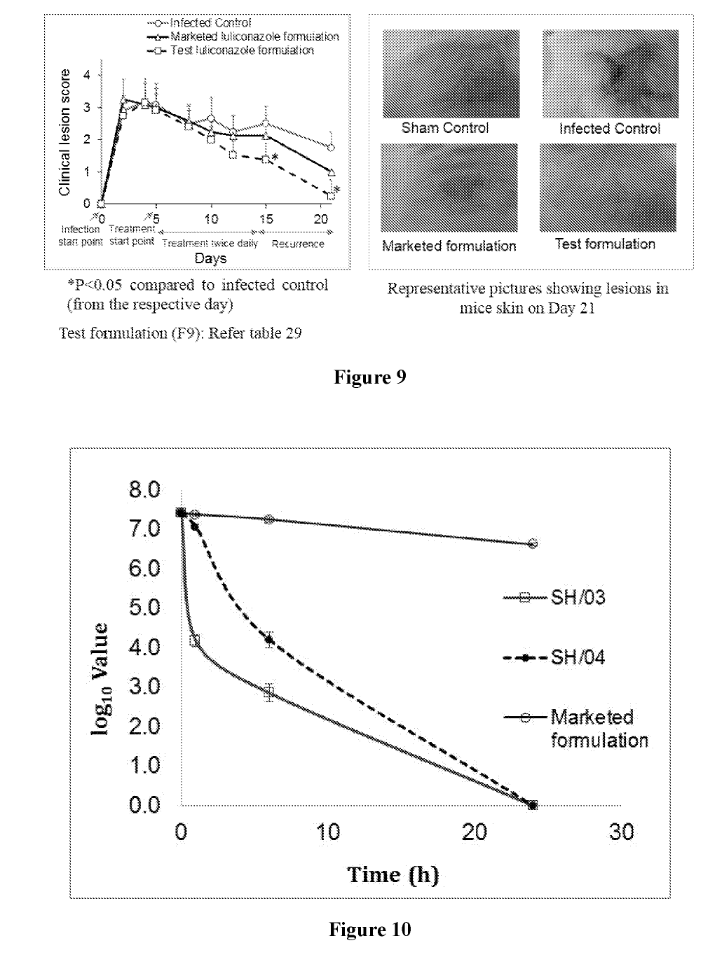

In vitro time kill assays were performed against M. furfur (MTCC 1374) using 100 times diluted ketoconazole shampoo formulations (SH/03, SH/04 and marketed 2% ketoconazole shampoo) in Sabouraud dextrose broth (SDB) containing 2% olive oil. The Malassezia inoculum was adjusted to achieve a cell density of approximately 10⁷ CFU/ml. The cells were incubated at (32±2°) C for 1, 6 and 24 h. At the end of each incubation period, cells were serially diluted and plated on Sabouraud dextrose agar (SDA) plates containing 2% olive oil. Plates were incubated at (32±2°) C for 3-4 days following which the total colony forming units (CFU) were enumerated. Experiment was performed in triplicates and data was plotted as changes in number of CFU over time (FIG. 10).

Results:

The study shows enhanced efficacy of present shampoo formulations containing ketoconazole (2%) and ester derivatives of caprylic acid [SH/03, SH/04] compared to marketed ketoconazole shampoo formulation.

### Example 26: Preparation of Hair Serum Formulations Containing an Antifungal Agent with at Least One or Two Medium Chain Fatty Acids and Derivatives Thereof (Table 36)

Procedure:
1) Phase A ingredients were weighed together and heated to 65-70° C.
2) Phase B ingredients were weighed and heated to 65-70° C.
3) Phase A was added to phase B slowly with continuous stirring at 700 rpm
4) Resulting mixture of phase A and phase B slowly cooled to 40° C.
5) Add phase C ingredients to the mixture of phase A and phase B with continuous stirring at 700 rpm
6) pH was adjusted to 5.5-6.5 by the addition of pH modifier

All the exemplary formulations HS1-HS4 mentioned in Table 36 are prepared using the above stated method.

TABLE 36
Exemplary hair serum formulations containing an antifungal agent with
at least one or two medium chain fatty acids and derivatives thereof

| | | Composition (% wt/wt) | | | |
|---|---|---|---|---|---|
| Phases | Ingredients | HS1 | HS2 | HS3 | HS4 |
| A | Piroctone olamine | 0.06 | 0.06 | 0.06 | 0.06 |
| | Propylene glycol | 4 | 4 | 4 | 4 |
| | caprylate | | | | |
| | PEG-12 dimethicone | 3 | 3 | 5 | 5 |
| | Oleth-20 | 3 | 4 | 4 | 3 |
| | Laureth 23 | 4 | 3 | NA | NA |
| B | Water | qs to | qs to | qs to | qs to |
| | | 100 | 100 | 100 | 100 |
| C | Sensomer CI 50 | NA | 0.2 | 0.2 | 0.2 |
| | PEG-120 methyl | 4 | NA | NA | 1 |
| | glucose trioleate | | | | |
| | Phenoxyethanol | 0.7 | 0.7 | 0.7 | 0.7 |
| | Fragrance | 0.5 | 0.5 | 0.5 | 0.5 |
| D | pH modiiter | qs to pH | qs to pH | qs to pH | qs to pH |

### Example 27: Preparation of Antimicrobial Body Lotion Formulations Containing an Antifungal Agent with at Least One or Two Medium Chain Fatty Acids and Derivatives Thereof (Table 37)

Procedure:
1) Phase A ingredients were weighed together and heated to 65-70° C.
2) Phase B ingredients were weighed together and heated to 65-70° C.
3) Phase A ingredients were added to phase B ingredients slowly with continuous stirring at 700 rpm.
4) Slowly cool the mixture of phase A and phase B to 40° C.
5) Phase C ingredients were weighed and added to the mixture of phase A and Phase B (Step 4).
6) Adjust the pH of resulting formulations to 5.5-6.5 using phase D.

All the exemplary formulations BLF1-BLF4 mentioned in Table 37 are prepared using the above stated method.

TABLE 37
Exemplary body lotion formulations containing an antifungal agent with
at least one or two medium chain fatty acids and derivatives thereof

| | | Composition (% wt/wt) | | | |
|---|---|---|---|---|---|
| Phases | Ingredients | BLF1 | BLF2 | BLF3 | BLF4 |
| A | Piroctone Olamine | 0.06 | 0.06 | 0.06 | 0.06 |
| | Propylene glycol | 5 | 5 | 5 | 5 |
| | monocarpylate | | | | |
| | Cetearyl alcohol | 4 | 4 | 3 | 4 |
| | Dimethicone | 5 | 5 | 2 | 2 |
| | Steareth 2 | 3.65 | 3.65 | 3.65 | 3.65 |
| | Steareth 21 | 0.35 | 0.35 | 0.35 | 0.15 |
| | Tocopherol acetate | 0.5 | 0.5 | 0.5 | 0.5 |
| B | Glycerin | 5 | 5 | 5 | 5 |
| | N-acetyl DGA | 5 | 5 | 5 | 5 |
| | Squalene | 2 | 7 | 2 | 2 |
| | Sodium hyaluronate | 0.2 | NA | 0.2 | 0.2 |
| | Xanthan gum | 0.3 | 0.3 | 0.5 | 0.5 |
| | Water | q.s. to | q.s. to | q.s. to | q.s. to |
| | | 100 | 100 | 100 | 100 |
| C | Cyclomethicone | 5 | 5 | 5 | 3 |
| | Phenoxyethanol | 1 | 1 | 1 | 1 |
| | Mentha Piperita | 2 | 2 | 2 | 2 |
| | Extract | | | | |
| | Fragrance | 0.5 | 0.5 | 0.5 | 0.5 |
| D | pH modifier | q.s. to | q.s. to | q.s. to | q.s. to |
| | | pH | pH | pH | pH |

### Example 28: Preparation of Formulations for Coating Surgical Implants Containing an Antifungal Antimicrobial Agent with at Least One or Two Medium Chain Fatty Acids and Derivatives Thereof (Table 38)

Method of Preparation (CCF1):

(1) Clotrimazole is solubilized in propylene glycol monocaprylate.

(2) The above solution was mixed in silicone based medical fluid at right proportion.

(3) Catheter is dipped into the coating solution for 3 minutes and the excess coating solution was allowed to drain by hanging them with proper support.

(4) Catheter is hanged with a clip or holder at 25° C. temperature and 55% RH for 24 h.

(5) The coated catheter is than sterilized and packed for further use.

The exemplary implant coating formulations CCF2-CCF6 with the respective compositions as mentioned in Table 38 are prepared using similar method as used for CCF1.

TABLE 38
Exemplary formulations for coating surgical implants containing an antimicrobial
agent with at least one or two medium chain fatty acids and derivatives thereof.

| | General | General | Composition | | | | | |
|---|---|---|---|---|---|---|---|---|
| Agents | characteristics | composition | CCF1 | CCF2 | CCF3 | CCF4 | CCF5 | CCF6 |
| Clotrimazole | Antimicrobial agent | 0.1-4 | 2 | 2 | | 2 | | 2 |
| Caprylic acid | Medium chain | 0.01-1 | | | 0.5 | | 0.5 | |
| | saturated and | | | | | | | |
| | unsaturated fatty | | | | | | | |
| | acids (C-1 to C-14) | | | | | | | |
| Propylene | Medium chain | 0.1-10 | 8 | 1 | 3 | 8 | 5 | 3 |
| glycol | saturated and | | | | | | | |
| monocaprylate | unsaturated fatty | | | | | | | |
| | acids ester and | | | | | | | |
| | derivatives (C-1 to | | | | | | | |
| | C-14) | | | | | | | |
| PEG-12- | Emulsifier/solubilizer | 0.1-10 | | | | 2 | 4 | 2 |
| Dimethicone | | | | | | | | |
| Hydroxyethyl | Hydrogel as coating | q.s. | | | | | | q.s. |
| cellulose | material | | | | | | | |
| Silicone based | Silicone based | q.s | q.s. | q.s. | q.s. | q.s. | q.s. | |
| medical fluid | coating material | | | | | | | |

Thus, extensive studies were carried out as described in the above examples which shows that the present compositions/formulations devoid of higher chain fatty acids/esters (greater than C14 or more) and containing medium chain fatty acids (C-1 to C-14) and/or esters thereof with antifungal agents (optionally along with excipients) show improved antifungal activity. Further, said combinations/compositions were shown to possess improved/synergistic activity also against drug resistant fungi.

1. An antifungal composition comprising at least one antifungal agent, at least one fatty acid or ester thereof, and optionally one or more excipient, wherein the antifungal agent is selected from the group consisting of allylamines, benzylamines, azoles, N-hydroxy pyridone, N-hydroxy pyrithione or metal coordination complexes and combinations thereof; wherein the fatty acid has a carbon chain length ranging from C-1 to C-14; and wherein the composition has synergistic antifungal activity.

2.-64. (canceled)

65. The antifungal composition of claim 1, wherein the fatty acid or ester thereof is a saturated or unsaturated fatty acid or ester of said saturated or unsaturated fatty acid; and wherein the fatty acid has a carbon chain length ranging from C-11 to C-14, or C-1 to C-10.

66. The antifungal composition of claim 1, wherein the fatty acid is selected from the group consisting of formic acid (C1), acetic acid (C2), propionic acid (C3), butyric acid (C4), valeric acid (C5), caproic acid (C6), enanthic acid (C7), caprylic acid (C8), pelargonic acid (C9), capric acid (C10), undecylic acid (C11), lauric acid (C12), tridecylic acid (C13), myristic acid (C14) and corresponding unsaturated fatty acids thereof.

67. The antifungal composition of claim 1, wherein the fatty acid ester is selected from the group consisting of propylene glycol monocaprylate, propylene glycol monolaurate, propylene glycol monocaprate, glyceryl monocaprylate, glyceryl monolaurate, glyceryl monocaprate, glyceryl dicaprylate, glyceryl dilaurate, glyceryl dicaprate, glyceryl mono and di caprate, glyceryl mono and di caprylate, glyceryl mono and di caprate caprylate, glyceryl mono and di laurate, triglycerides of caprylic acid, capric acid, lauric acid and their mixtures, and combinations thereof, preferably propylene glycol monocaprylate, glyceryl mono-di caprate, or glyceryl mono-di caprylate.

68. The antifungal composition of claim 1, wherein the N-hydroxy pyridone is piroctone olamine, ciclopirox olamine or a combination thereof; the N-hydroxy pyrithione or the metal coordination complex is zinc pyrithione or any respective bivalent metal coordinating complexes or combinations thereof; allylamines are selected from the group consisting of terbinafine, amorolfine, naftifine and combinations thereof; the benzylamine is butenafine; and the azoles are imidazoles, triazoles or thiazoles selected from the group consisting of ketoconazole, climbazole, miconazole nitrate, fluconazole, econazole, saperconazole, oxiconazole, clotrimazole, bifonazole, butoconazole, fenticonazole, isoconazole, omoconazole, sertaconazole, sulconazole, tioconazole, luliconazole, chlormidazole, croconazole, eberconazole, omoconazole, isoconazole, neticonazole, albaconazole, efinaconazole, fosfluconazole, epoxiconazole, fluconazole, isavuconazole, itraconazole, posaconazole, propiconazole, ravuconazole, terconazole, voriconazole, hexaconazole, abafungin and combinations thereof.

69. The antifungal composition of claim 1, wherein the excipient is selected from the group consisting of additive, solvent, oil, emulsifier, surfactant, stabilizer, cooling agent, preservative, antioxidant, gelling agent, moisturizing agent, emollient, penetration enhancer, colorant, fragrance, pH modifiers, conditioning agent, pearlizing agents, skin barrier repair agents, and combinations thereof; and wherein the additive selected from the group consisting of paraffin, thickeners selected from bentonite and cellulose, antioxidants selected from butylated hydroxyanisole (BHA), tert-butylhydroquinone (TBHQ), ferulic acid, tocopherol acetate or any combination of antioxidants thereof, perfumes or fragrances, essential oils, pH adjusters selected from triethanolamine, sodium hydroxide, inorganic or organic acids including citric acid, lactic acid, succinic acid, acetic acid, fumaric acid, glycolic acid, benzoic acid, bases, salts buffers or any combination of pH adjusters thereof, herbal extracts selected from amla fruit extract, arnica extract and brahmi extract, preserving agents selected from butylated hydroxytoluene (BHT), methyl p-hydroxybenzoate, propyl p-hydroxybenzoate, sorbic acid or any combination of preserving agents thereof, hair conditioning substances, hair care adjuncts selected from taurine, caffeine, minoxidil, azelaic acid, marine cartilage, hydrolysed keratin, biotin, niacin, panthenol, vitamin B6, zinc, copper, peptides, horsetail silica, beta sitosterols, pycnogenol, PABA, green tea extract, folic acid, iron, L-cysteine, magnesium, ginseng or any combination of hair care adjuncts thereof, skin care adjuncts selected from proteins, vitamins including A, B, C, D, E and K, trace metals including zinc, calcium and selenium, moisturizers, LTV absorbers including paraminobenzoic acid (PABA), titanium dioxide, zinc oxide, anti-irritants including steroids and non-steroidal anti-inflammators, botanical extracts including aloe vera, chamomile, cucumber extract, ginkgo biloba, ginseng and rosemary, absorbents including aluminum starch octenylsuccinate, kaolin, corn starch, oat starch, cyclodextrin, talc and zeolite, skin bleaching and lightening agents including hydroquinone and niacinamide lactate, humectants including sorbitol, urea and manitol, exfoliants, cooling agents selected from menthol, menthol derivatives, WS 3, WS-5, WS 14, WS 23, MHB, frescolat MGA, 2S MPD, coolcat P, WS 30, PM 38, skin conditioning agents selected from aloe extract, allantoin, bisabolol, shea butter, ceramides, sphingosine, dimethicone, hyaluronic acid and dipotassium glycyrrhizate, natural components including oatmeal, or any combination of skin care adjuncts thereof, emollient, dyestuffs, moisturizers, vitamins, sphingoceryls, sunscreens, co-surfactants, foaming agents, co-emulsifiers, viscosity modifiers, suspending agents, potentiating agents, pearlizing agents, cooling agents, ionic strength modifiers and oil-soluble polymers which are compatible with the base oil or skin care agents or both including skin-nutrient agents, anti-wrinkle agents, light and dust protectors, and combinations thereof; solvent selected from the group consisting of ethanol, isopropyl alcohol, butanol, C-1 to C-6 lower aliphatic alcohols, lower alkyl acetate, ethers, carboxylic acid, derivatives containing carbon chain length less than C15, fatty alcohols selected from the group consisting of undecanol, oleyl alcohol and lauryl alcohol, or any combination of solvents thereof; emulsifier selected from the group consisting of steareth-2, steareth-21, poloxamer, macrogolcetostearyl ether 20, cetyl alcohol cetearths, ceteth, isoceteths, laureths, oleths, steareths, lauramide DEA, linoleamide DEA or any combination of emulsifiers thereof; surfactant selected from the group consisting of poloxamer, PEG-2 stearyl ether, PEG-21 stearyl ether, pluoronic F127 (poloxamer), polyoxyl 20 cetosteryl ether, sodium laryl ether sulphate, coco monoethanolamide, cocamidopropylbetain, sodium docusate, ammonium lauryl sulphate, coco glucoside, lauryl glucoside, decyl glucoside, caprylyl capryl glucoside, sodium cocoyl glutamate, disodium cocoyl glutamate, sodium lauroamphoacetate, sodium cocoamphoacetate, disodium cocoamphoacetate, disodium laureth sulfosuccinate, sodium methyl cocoyl taurate, sodium methyl oleoyl taurate, sodium cocoyl isethionate, ammonium cocoyl isethionate, sodium lauryl glucose carboxylate, sodium lauroyl lactylate, sodium lauroyl sarcosinate, sodium lauroyl methyl isethionate, sodium cocoyl glycinate, or any combination of surfactants thereof; surfactant and co-surfactant blend selected from Iselux SLC consisting of sodium lauroyl methyl isethionate, sodium lauroamphoacetate, cocamide MIPA and water, Miracare SLB 365/N consisting of sodium trideceth sulfate, sodium lauroamphoacetate, cocamide MEA, sodium chloride, methylisothiazolinone and water, or a combination of blend thereof; oil including natural or synthetic oils selected from the group consisting of eucalyptus oil, rosemary oil, pine needle oil, tea tree oil, sage oil, cinnamon oil, lemon oil, citronella oil, lime oil, orange oil, peppermint oil, spearmint oil, wintergreen oil, sweet birch oil, clove leaf oil, camphor oil, cardamon oil, cedar leaf oil, sweet birch oil, paraffin oil, silicone oil or any combination of oils thereof; polymer selected from the group consisting of PEG, cellulose derivatives, acrylic based polymers, poloxamers, and combinations thereof; stabilizer selected from the group consisting of metal chelators, acrylic and cellulose derivatives, sodium carboxy methyl cellulose, poly vinyl alcohol, xanthan gum, guar gum, locust bean gum and combinations thereof; and active agent selected from the group consisting of pharmaceutical active, OTC active, anti-bacterial agent including benzothenium chloride, anti-inflammatory agent, skin penetration enhancer and combinations thereof.

70. The antifungal composition of claim 1, wherein the composition comprises about 0.01% to 20% by weight of the antifungal agent, preferably about 0.01% to 15% by weight of the antifungal agent.

71. The antifungal composition of claim 1, wherein the composition comprises about 0.01% to 30% by weight of the saturated or unsaturated fatty acid or ester thereof, preferably about 0.01% to 20% by weight of the saturated or unsaturated fatty acid or ester thereof.

72. The antifungal composition of claim 1, wherein the composition comprises about 45% to 99% by weight of the excipient, preferably about 80% to 99% by weight of the excipient.

73. The antifungal composition of claim 1, wherein the composition comprises propylene glycol monocaprylate and antifungal agent selected from the group consisting of terbinafine, butenafine, clotrimazole, ketoconazole, luliconazole, bifonazole, efinaconazole, zinc pyrithione, piroctone olamine, ciclopirox olamine and combinations thereof, and optionally at least one excipient.

74. The antifungal composition of claim 1, wherein the composition comprises: (a) piroctone olamine, propylene glycol monocaprylate and at least one excipient, (b) zinc pyrithione, propylene glycol monocaprylate and at least one excipient, (c) ketoconazole, propylene glycol monocaprylate and at least one excipient, (d) ketoconazole, zinc pyrithione, propylene glycol monocaprylate and at least one excipient, (e) clotrimazole, propylene glycol monocaprylate and at least one excipient, (f) luliconazole, propylene glycol monocaprylate and at least one excipient, (g) terbinafine, propylene glycol monocaprylate and at least one excipient, or (h) efinaconazole, propylene glycol monocaprylate and at least one excipient.

75. The antifungal composition of claim 1, wherein the composition comprises: (a) clotrimazole, (b) propylene glycol monocaprylate, and at least one excipient; or (a) luliconazole, (b) propylene glycol monocaprylate, and at least one excipient; or (a) terbinafine, (b) propylene glycol monocaprylate, and at least one excipient; or (a) efinaconazole, (b) propylene glycol monocaprylate, and at least one excipient; or (a) ketoconazole, (b) zinc pyrithione, (c) propylene glycol monocaprylate, and at least one excipient; or (a) piroctone olamine, (b) propylene glycol monocaprylate, and at least one excipient, wherein said excipient comprises benzothenium chloride.

76. The antifungal composition of claim 1, wherein the composition comprises: (a) allylamine or benzylamine antifungal agent, undecylenic acid, and at least one excipient, wherein the allylamine or benzylamine is terbinafine, butenafine or a combination thereof, (b) allylamine or benzylamine antifungal agent, undecylenic acid ester, and at least one excipient, wherein the allylamine or benzylamine is terbinafine, butenafine or a combination thereof, (c) allylamine or benzylamine antifungal agent, undecylenic acid or it's ester, at least one antifungal selected from clotrimazole, luliconazole, efinaconazole and piroctone olamine, and at least one excipient (d) allylamine or benzylamine, propylene glycol monocaprylate, clotrimazole, and at least one excipient (e) allylamine or benzylamine, propylene glycol monocaprylate, luliconazole, and at least one excipient, (f) allylamine or benzylamine, propylene glycol monocaprylate, efinaconazole, and at least one excipient, (g) allylamine or benzylamine, propylene glycol monocaprylate, ketoconazole, zinc pyrithione, and at least one excipient, (h) allylamine or benzylamine, propylene glycol monocaprylate, piroctone olamine, and at least one excipient, (i) allylamine or benzylamine, undecylenic acid or it's ester, an imidazole selected from ketoconazole, clotrimazole and luliconazole, and at least one excipient, or (j) allylamine or benzylamine, a triazole, a fatty acid selected from propylene glycol monocaprylate, caprylic acid, lauric acid, glyceryl monocaprylate, propylene glycol monolaurate and undecylenic acid, and at least one excipient.

77. The antifungal composition of claim 1, wherein the composition comprises: (a) imidazole antifungal agent, undecylenic acid, and at least one excipient, wherein the imidazole is ketoconazole, clotriconazole, luliconazole or any combination thereof, (b) ketoconazole, zinc pyrithione, propylene glycol monocaprylate, and at least one excipient, (c) imidazole antifungal agent, terbinafine, propylene glycol monocaprylate and at least one excipient, (d) imidazole antifungal agent, efinaconazole, propylene glycol monocaprylate, and at least one excipient, (e) imidazole antifungal agent, piroctone olamine, propylene glycol monocaprylate, and at least one excipient, (f) imidazole antifungal agent, an allylamine or benzylamine antifungal agent, undecylenic acid, and at least one excipient, (g) imidazole antifungal agent, triazole agent, at least one fatty acid selected from propylene glycol monocaprylate, caprylic acid, lauric acid, glyceryl monocaprylate, propylene glycol monolaurate and undecylenic acid, and at least one excipient, (h) ketoconazole, zinc pyrithione, propylene glycol monocaprylate, and at least one excipient, or (i) imidazole antifungal agent, a triazole antifungal agent, allylamine or benzylamine antifungal agent, undecylenic acid and at least one excipient.

78. The antifungal composition of claim 1, wherein the composition comprises: (a) triazole antifungal agent, at least one fatty acid or ester thereof selected from caprylic acid, capric acid, lauric acid, propylene glycol monocaprylate, glyceryl monocaprylate, propylene glycol monolaurate, undecylenic acid, and at least one excipient, (b) triazole antifungal agent, undecylenic acid or it's ester, and at least one excipient, (c) efinaconazole, at least one fatty acid selected from caprylic acid, lauric acid, capric acid and undecylenic acid or esters thereof, and at least one excipient, (d) efinaconazole, propylene glycol monocaprylate, and at least one excipient, (e) itraconazole propylene glycol monocaprylate, and at least one excipient, (f) triazole antifungal agent, clotrimazole propylene glycol monocaprylate, and at least one excipient, (g) triazole antifungal agent, luliconazole propylene glycol monocaprylate, and at least one excipient, (h) triazole antifungal agent, terbinafine, propylene glycol monocaprylate, and at least one excipient, (i) triazole antifungal agent, ketoconazole, zinc pyrithione propylene glycol monocaprylate, and at least one excipient, (j) triazole antifungal agent, piroctone olamine, propylene glycol monocaprylate, and at least one excipient, or (k) triazole antifungal agent, an allylamine or benzylamine antifungal agent, undecylenic acid, and at least one excipient.

79. The antifungal composition of claim 1, wherein the composition comprises: (a) N-hydroxy pyridone antifungal agent, clotrimazole, propylene glycol monocaprylate, and at least one excipient, (b) N-hydroxy pyridone antifungal agent, luliconazole, propylene glycol monocaprylate, and at least one excipient, (c) N-hydroxy pyridone antifungal agent, terbinafine, propylene glycol monocaprylate, and at least one excipient, (d) N-hydroxy pyridone antifungal agent, efinaconazole, propylene glycol monocaprylate, and at least one excipient, (e) N-hydroxy pyridone antifungal agent, ketoconazole, zinc pyrithione, propylene glycol monocaprylate, and at least one excipient, (f) N-hydroxy pyridone antifungal agent, allylamine or benzylamine antifungal agent, undecylenic acid, and at least one excipient, (f) N-hydroxy pyridone antifungal agent, imidazole antifungal agent, undecylenic acid, and at least one excipient, (g) N-hydroxy pyridone antifungal agent, triazole antifungal agent, at least one fatty acid selected from caprylic acid, lauric acid, propylene glycol monocaprylate, glyceryl monocaprylate, propylene glycol monolaurate and undecylenic acid or esters thereof, and at least one excipient, (h) ciclopirox, at least one fatty acid selected from glyceryl monocaprylate, propylene glycol monocaprylate, caprylic acid and undecylenic acid, and at least one excipient, (i) ciclopirox, clotrimazole, propylene glycol monocaprylate, and at least one excipient, (j) ciclopirox, luliconazole, propylene glycol monocaprylate, and at least one excipient, (k) ciclopirox, terbinafine, propylene glycol monocaprylate, and at least one excipient, (l) ciclopirox, efinaconazole, propylene glycol monocaprylate, and at least one excipient, (m) ciclopirox, ketoconazole, zinc pyrithione, propylene glycol monocaprylate, and at least one excipient, (n) ciclopirox, an allylamine or benzylamine antifungal agent, undecylenic acid, and at least one excipient, (o) ciclopirox, an imidazole antifungal agent, undecylenic acid, and at least one excipient, or (p) ciclopirox, triazole antifungal agent, at least one fatty acid selected from caprylic acid, lauric acid, propylene glycol monocaprylate, glyceryl monocaprylate, propylene glycol monolaurate and undecylenic acid or esters thereof, and at least one excipient.

80. The antifungal composition of claim 1, wherein the composition comprises: (a) N-hydroxy pyrithione or metal coordination complex antifungal agent, clotrimazole, propylene glycol monocaprylate, and at least one excipient, (b) N-hydroxy pyrithione or metal coordination complex antifungal agent, luliconazole, propylene glycol monocaprylate, and at least one excipient, (c) N-hydroxy pyrithione or metal coordination complex antifungal agent, terbinafine, propylene glycol monocaprylate, and at least one excipient, (d) N-hydroxy pyrithione or metal coordination complex antifungal agent, efinaconazole, propylene glycol monocaprylate, and at least one excipient, (e) ketoconazole, N-hydroxy pyrithione or metal coordination complex antifungal agent, propylene glycol monocaprylate, and at least one excipient, (f) N-hydroxy pyrithione or metal coordination complex antifungal agent, piroctone olamine, propylene glycol monocaprylate, and at least one excipient, (g) N-hydroxy pyrithione or metal coordination complex antifungal agent, allylamine or benzylamine antifungal agent, undecylenic acid, and at least one excipient, (h) N-hydroxy pyrithione or metal coordination complex antifungal agent, imidazole antifungal agent, undecylenic acid, and at least one excipient, (i) N-hydroxy pyrithione or metal coordination complex antifungal agent, triazole antifungal agent, a fatty acid selected from caprylic acid, lauric acid, propylene glycol monocaprylate, glyceryl monocaprylate, propylene glycol monolaurate, and undecylenic acid or esters thereof, and at least one excipient, or (j) N-hydroxy pyrithione or metal coordination complex antifungal agent, azole antifungal agent, caprylic acid, lauric acid, propylene glycol monocaprylate, glyceryl monocaprylate, propylene glycol monolaurate, and undecylenic acid or esters thereof, and at least one excipient.

81. The antifungal composition of claim 1, wherein the composition is formulated for topical administration, local administration, systemic administration, or any combination thereof; and wherein the composition is formulated into cream, oil, lotion, serum, gel, emugel, hydrogel, shampoo, nail varnish, ointment, foam, spray, aerosol, coating for material selected from surgical implants, silicon tube, catheter, valves, stents, or suture; or any combination of formulations thereof.

82. A method for treating a fungal infection in a subject in need thereof or managing fungal growth, comprising administering the antifungal composition of claim 1 to the subject, or contacting the antifungal composition of claim 1 with the fungus.

83. The method of claim 1, wherein the treating or managing comprises inhibiting the fungal growth, reducing the fungal growth, eliminating the fungus, curing drug resistant fungal infections, treatment of fungal infections in clinical non-responders and patients with barrier defects, or any combination thereof; wherein the treatment includes medical treatment, cosmetic treatment, or a combination thereof; wherein the fractional inhibitory concentration (FIC) index of the composition is less than 1; wherein the fungal infection or fungal growth is caused by Malassezia spp. selected from the group consisting of M. furfur, M. pachydermatis, M. globosa, M. restricta, M. slooffiae, M. sympodialis, M. nana, M. yamatoensis, M. dermatis, M. obtusa, M. japonica, M. caprae, M. cuniculi, M. equine, and M. arunalokei; Trichophyton spp. selected from the group consisting of T. rubrum, T. mentagrophyte, T. interdigitale, T. tonsurans, T. schoenleinii, T. violaceum, T. abissinicum, T. balcaneum, T. circonvolutum, T. concentricum, T. eboreum, T. errinacei, T. fischeri, T. fluviomuniense, T. glabrum, T. gourvilii, T. kanei, T. kuryangei, T. megninii, T. pedis, T. proliferans, T. raubitschekii, T. redellii, T. rodhainii, T. simii, T. soudanense, T. thuringiense, T. verrucosum, T. violaceum and Trichophyton yaoundei; Candida spp. selected from the group consisting of C. albicans, C. glabrata, C. guilliermondii, C. krusei, C. lusitaniae, C. parapsilosis, C. tropicalis, C. colliculosa, C. dubliniensis, C. famata, C. haemulonii, C. inconspicua, C. intermedia, C. kefyr, C. lipolytica, C. metapsilosis, C. norvegensis, C. orthopsilosis, C. pelliculosa, C. pulcherrima, C. rugose, C. utilis, C. viswanathii, and C. zeylanoides; Microsporum spp. selected from the group consisting of M. audouinii, M. canis, M. amazonicum, M. boullardii, M. cookie, M. distortum, M. duboisii, M. equinum, M. ferrugineum, M. fulvum, M. gallinae, M. gypseum, M. langeronii, M. nanum, M. persicolor, M. praecox, M. ripariae and M. rivalieri; Epidermaphyton spp such as E. floccosum; and other non-dermatophytes including but not limited to Aspergillus spp. selected from the group consisting of A. fumigates, A. flavus, A. nidulans, A. terreus, A. lentulus, A. niger, A. alliaceus, A. arvii, A. brevipes, A. calidoustus, A. conjunclus, A. deflectus, A. duricaulis, A. emericella, A. fischerian, A. fumigatiainis, A. fumisynnematus, A. granulosus, A. novofimigatus, A. panamensis, A. quadrilineatus, A. udagawae, A. unilateralis and A. ustus; and Cryptococcus spp. selected from the group consisting of C. neoformans, C. gattii, C. albidus, C. bacillisporus, C. decagatti, C. deuterogatti, C. laurentii, C. tetragatti and C. uniguttulatus; or any combination of fungi thereof; wherein the fungus is resistant or susceptible to the antifungal agent comprised in the antifungal composition; and wherein the subject is mammal including human.
